(19) United States
(12) Patent Application Publication
(10) Pub. No.: US 2021/0138249 A1
(43) Pub. Date: May 13, 2021

(54) SYSTEM AND METHOD FOR NEURAL STIMULATION USING SPIKE FREQUENCY MODULATION

(71) Applicant: Newton Howard, Providence, RI (US)

(72) Inventor: Newton Howard, Providence, RI (US)

(21) Appl. No.: 16/944,963

(22) Filed: Jul. 31, 2020

Related U.S. Application Data
(63) Continuation-in-part of application No. 15/988,315, filed on May 24, 2018.
Application No. 15/988,315 is a continuation-in-part of application No. 15/495,959, filed on Apr. 24, 2017.
Application No. 15/495,959 is a continuation-in-part of application No. 16/545,205, filed on Aug. 20, 2019.
Application No. 16/545,205 is a continuation-in-part of application No. 16/785,969, filed on Feb. 10, 2020.
(60) Provisional application No. 62/326,007, filed on Apr. 22, 2016.
(60) Provisional application No. 62/353,343, filed on Jun. 22, 2016.
(60) Provisional application No. 62/397,474, filed on Sep. 21, 2016.
(60) Provisional application No. 62/511,532, filed on May 26, 2017.
(60) Provisional application No. 62/534,671, filed on Jul. 19, 2017.
(60) Provisional application No. 62/560,750, filed on Sep. 20, 2017.
(60) Provisional application No. 62/658,764, filed on Apr. 17, 2018.
(60) Provisional application No. 62/665,611, filed on May 2, 2018.
(60) Provisional application No. 62/719,849, filed on Aug. 20, 2018.
(60) Provisional application No. 62/783,050, filed on Dec. 20, 2018.
(60) Provisional application No. 62/726,699, filed on Sep. 4, 2018.
(60) Provisional application No. 62/881,220, filed on Jul. 31, 2019.
(60) Provisional application No. 62/883,983, filed on Aug. 7, 2019.
(60) Provisional application No. 62/896,563, filed on Sep. 5, 2019.
(60) Provisional application No. 62/896,571, filed on Sep. 5, 2019.
(60) Provisional application No. 62/912,515, filed on Oct. 8, 2019.

(51) Int. Cl. A61N 1/36; A61N 5/06; A61N 1/372; G16H 20/30

(52) U.S. Cl. CPC A61N 1/36139; A61N 5/0622; A61N 1/36067; G16H 20/30; A61N 1/36071; A61N 1/37252; A61N 1/36082

(57) ABSTRACT

Embodiments may comprise receiving electrical and optical signals from electrophysiological neural signals of neural tissue from at least one read modality, wherein the electrophysiological neural signals are at least one of Spike frequency modulated or Spike frequency demodulated, encoding the received electrical and optical signals using a Fundamental Code Unit, automatically generating at least one machine learning model using the Fundamental Code Unit encoded electrical and optical signals, generating at least one optical or electrical signal to be transmitted to the brain tissue using the generated at least one machine learning model, wherein the generated signals are at least one of Spike frequency modulated or Spike frequency demodulated, and transmitting the generated at least one optical or electrical signal to the neural tissue to provide electrophysiological stimulation of the neural tissue using at least one write modality.

### CROSS-REFERENCE TO RELATED APPLICATIONS

This application claims the benefit of U.S. Provisional App. No. 62/881,220, filed Jul. 31, 2019, U.S. Provisional App. No. 62/883,983, filed Aug. 7, 2019, U.S. Provisional App. No. 62/896,563, filed Sep. 5, 2019, U.S. Provisional App. No. 62/896,571, filed Sep. 5, 2019, and U.S. Provisional App. No. 62/912,515, filed Oct. 8, 2019, and is a continuation-in-part of U.S. application Ser. No. 16/785,969, filed Feb. 10, 2020, which claims the benefit of U.S. Provisional App. No. 62/803,491, filed Feb. 9, 2019, and which is a continuation-in-part of U.S. application Ser. No. 15/988,315, filed May 24, 2018, which claims the benefit of U.S. Provisional App. No. 62/665,611, filed May 2, 2018, U.S. Provisional App. No. 62/658,764, filed Apr. 17, 2018, U.S. Provisional App. No. 62/560,750, filed Sep. 20, 2017, U.S. Provisional App. No. 62/534,671, filed Jul. 19, 2017, and U.S. Provisional App. No. 62/511,532, filed May 26, 2017, which is a continuation-in-part of U.S. application Ser. No. 15/495,959, filed Apr. 24, 2017, which claims the benefit of U.S. Provisional App. No. 62/326,007, filed Apr. 22, 2016, U.S. Provisional App. No. 62/353,343, filed Jun. 22, 2016, and U.S. Provisional App. No. 62/397,474, filed Sep. 21, 2016, and is a continuation-in-part of U.S. application Ser. No. 16/545,205, filed Jul. 24, 2019, which claims the benefit of U.S. Provisional App. No. 62/783,050, filed Dec. 20, 2018, U.S. Provisional App. No. 62/726,699, filed Sep. 4, 2018, and U.S. Provisional App. No. 62/719,849, filed Aug. 20, 2018, the contents of all of which are incorporated herein in their entirety.

### BACKGROUND

The present invention relates to techniques for brain interfacing, mapping neuronal structure (Google earth for brains), manipulating cellular structure, cognitive, and brain augmentation via implants, and curing, not just managing, neurological disorders.

According to the United Nations, roughly one billion people, nearly ⅙th of the world's population, presently suffer from some form of neurological disorder, with some 6.8 million deaths each year. During the past decades, a large amount of work on several brain diseases were unsuccessful, because they take neither the initial state of the neuronal—brain region nor the initial neuronal interplay into consideration, greatly limiting the validity of conclusions made. Because the data recorded are only a snapshot of a precise situation, conclusions must be made based mainly on assumption about the properties of neurons and networks.

The UN estimates that one in every four people will suffer from a neurological or mental disorder in their lifetime and the vast majority of these cases will remain undiagnosed. Of those who are diagnosed, the World Health Organization claims two-thirds never seek treatment (reference). Conventional systems cannot quantitatively detect and track the progression of a neurological disease (or the efficacy of a treatment).

The insertion of brain implants for neural monitoring or stimulation may lead to considerable scar tissue formation at the site of implant. The extent of the scar tissue scales with cortical tissue damage, caused directly by sharp non-compliant brain probes, or by straining the tissue by large volume implant. Both of these issues constrain the size and therefore the number of electrical brain-probing sites that may be embedded on brain probes since excessive scar tissue insulates the probe from the local neuronal environment degrading the electrical signals.

Accordingly, a need arises for a system that can quantitatively detect and track the progression of a neurological disease (or the efficacy of a treatment), provide the capability to receive neuronal signals from brain tissue and to transmit signals to brain tissue, as well as local and network-based processing to analyze and generate such signals, and enable long term use of such implants.

### SUMMARY

Embodiments of the present invention may provide techniques for brain interfacing, mapping neuronal structure (Google earth for brains), manipulating cellular structure, cognitive, and brain augmentation via implants, and curing, not just managing, neurological disorders. Embodiments may utilize the Fundamental Code Unit (FCU) of the Brain, or Brain Code. The FCU may map higher-order cognitive and behavioral processes to observed neurological states. For example, healthy vs diseased functions and tissues may be mapped, as a lack of function indicates circuits that may be diseased. In addition, embodiments may utilize mapping New technologies such as voltage-sensitive organic dyes and Quantum Dots (QDs).

Embodiments may include two main functional/structural elements—the BrainOS Engine and the KIWI implantable neural sensor and stimulation device. The BrainOS, described further below, may include functional elements such as a Deep Cognitive Neural Network (DCNN) and a solution Architecture, as described below. The Deep Cognitive Neural Network (DCNN) architecture may integrate both convolutional feedforward and recurrent network principles, and may employ a novel queuing theory driven design to create perception and reasoning characteristics similar to the human brain.

In an embodiment, a method for neural stimulation may comprise receiving electrical and optical signals from electrophysiological neural signals of neural tissue from at least one read modality, wherein the electrophysiological neural signals are at least one of Spike frequency modulated or Spike frequency demodulated, encoding the received electrical and optical signals using a Fundamental Code Unit, automatically generating at least one machine learning model using the Fundamental Code Unit encoded electrical and optical signals, generating at least one optical or electrical signal to be transmitted to the brain tissue using the generated at least one machine learning model, wherein the generated signals are at least one of Spike frequency modulated or Spike frequency demodulated, and transmitting the generated at least one optical or electrical signal to the neural tissue to provide electrophysiological stimulation of the neural tissue using at least one write modality.

In embodiments, the received Spike frequency modulated signals may be obtained from sensory neurons. The generated Spike frequency modulated signals may be generated using signal transform function that converts an analog stimulus on a sensory neuron into a sequence of spikes, wherein the rate of spikes per second (sps) is proportional to the intensity of the input. The generated Spike frequency modulated signals may have a rate of 0 to 100 spikes per second and an amplitude of 0 to 100 mV. The received Spike frequency demodulated signals may be obtained from motor neurons. The generated Spike frequency demodulated signals may be generated using a left rectangular numerical integration of the SFM signals for each sampling period determined by a given threshold of conversion.

In an embodiment, a system for neural stimulation may comprise at least one read modality adapted to receive electrical and optical signals from electrophysiological neural signals of neural tissue, wherein the electrophysiological neural signals are at least one of Spike frequency modulated or Spike frequency demodulated, at least one write modality adapted to transmit the generated at least one optical or electrical signal to the neural tissue to provide electrophysiological stimulation of the brain tissue, and at least one computing device comprising a processor, memory accessible by the processor, and program instructions stored in the memory and executable by the processor to cause the processor to perform: encoding the received electrical and optical signals using a Fundamental Code Unit, automatically generating at least one machine learning model using the Fundamental Code Unit encoded electrical and optical signals, and generating at least one optical or electrical signal to be transmitted to the neural tissue using the generated at least one machine learning model, wherein the generated signals are at least one of Spike frequency modulated or Spike frequency demodulated.

In an embodiment, a computer program product may comprise a non-transitory computer readable storage having program instructions embodied therewith, the program instructions executable by a computer system, to cause the computer system to perform a method of neural stimulation comprising: receiving electrical and optical signals from electrophysiological neural signals of neural tissue from at least one read modality, wherein the electrophysiological neural signals are at least one of Spike frequency modulated or Spike frequency demodulated, encoding the received electrical and optical signals using a Fundamental Code Unit, automatically generating at least one machine learning model using the Fundamental Code Unit encoded electrical and optical signals, generating at least one optical or electrical signal to be transmitted to the brain tissue using the generated at least one machine learning model, wherein the generated signals are at least one of Spike frequency modulated or Spike frequency demodulated, and transmitting the generated at least one optical or electrical signal to the neural tissue to provide electrophysiological stimulation of the neural tissue using at least one write modality.

### BRIEF DESCRIPTION OF THE DRAWINGS

The details of the present invention, both as to its structure and operation, can best be understood by referring to the accompanying drawings, in which like reference numbers and designations refer to like elements.

FIG. 1 is an exemplary illustration of a theoretical framework for understanding healthy brain function and the brain's capacity for intelligent action.

FIG. 2 is an exemplary block diagram of the Fundamental Code Unit (FCU) of the Brain, or Brain Code.

FIG. 3 is an exemplary block diagram of a BrainOS AI Engine.

FIG. 4 is an exemplary illustration of BrainOS Use Cases.

FIG. 5 is an exemplary illustration of BrainOS Architecture.

FIG. 6 is an exemplary illustration of a Wellness Use Case.

FIG. 7 is an exemplary illustration of a neuropsin-controlled, cGMP-mediated transduction cascade cycle.

FIG. 8 is an exemplary illustration of an implantable sensor system.

FIG. 9 illustrates an exemplary embodiment of a Biological Co-Processor System (BCP).

FIG. 10 illustrates an exemplary embodiment of an implantable signal receiving, processing, and transmitting device, shown in FIG. 9.

FIG. 11 illustrates an exemplary embodiment of Brain Code Collection System earbud, shown in FIG. 9.

FIG. 12 illustrates an exemplary embodiment of a cloud platform.

FIG. 13 illustrates an exemplary embodiment of an inductive powering system.

FIG. 14 illustrates exemplary advantages of aspects of technologies that may be utilized by embodiments.

FIG. 15 illustrates exemplary advantages of aspects of technologies that may be utilized by embodiments.

FIG. 16 illustrates an exemplary embodiment of an implant device.

FIG. 17 illustrates an exemplary embodiment of an implant device.

FIG. 18 illustrates an exemplary embodiment of a tile design for an implant device.

FIG. 19 illustrates an exemplary embodiment of a tile arrangement for an implant device.

FIG. 20 is an exemplary illustration of an approximate representation of how the optrode array could fit over a dense neural network.

FIG. 21 illustrates an exemplary embodiment of an implant device.

FIG. 22 illustrates an exemplary embodiment of an implant device.

FIG. 23 illustrates an exemplary embodiment of CNT connection for an implant device.

FIG. 24 illustrates an example of fast-scan cyclic voltammetry.

FIG. 25 illustrates an example of how carbon nanotube color changes with chiral index.

FIG. 26 illustrates an exemplary embodiment of a nanoengineered electroporation microelectrodes (NEM).

FIG. 27 illustrates an exemplary embodiment of an electrophysiological recording pipeline.

FIG. 28 illustrates an exemplary embodiment of an optical recording pipeline.

FIG. 29 illustrates an exemplary embodiment of an optical recording pipeline.

FIG. 30 illustrates an example of cyclically applied potential for cyclic voltammetry.

FIG. 31 illustrates an exemplary embodiment of recording pipelines and data processing circuitry.

FIG. 32 illustrates an example of spike trains of ChR2 and NpHR expressing neurons when subjected to light beams of different wavelengths.

FIG. 33 illustrates an example of Poisson trains of spikes elicited by pulses of blue light (dashes), in two different neurons.

FIG. 34 illustrates examples of a light-driven spike blockade for different neurons.

FIG. 35 illustrates examples of reaction events for different neurons.

FIG. 36 examples of the correlation between wavelengths (nm) and normalized cumulative charge for different Channelrhodopsins neurons.

FIG. 37 illustrates an exemplary embodiment of an optical stimulation pipeline.

FIG. 38 illustrates an exemplary embodiment of an optical stimulation pipeline.

FIG. 39 illustrates an exemplary embodiment of an optical stimulation pipeline.

FIG. 40 illustrates an exemplary embodiment of optical stimulation pipelines.

FIG. 41 illustrates an exemplary embodiment of an implant device.

FIG. 42 illustrates an exemplary embodiment of pseudocode for a process of data recording.

FIG. 43 illustrates an exemplary embodiment of pseudocode for a process of stimulation requests.

FIG. 44 illustrates an exemplary embodiment of a closed loop control system.

FIG. 45 illustrates an exemplary embodiment of pseudocode for a closed loop control system.

FIG. 46 illustrates an exemplary embodiment of pseudocode for a PID algorithm.

FIG. 47 illustrates exemplary data flow block diagram of a spike sorting technique.

FIG. 48a illustrates a portion of an exemplary embodiment of pseudocode for performing an SPC method.

FIG. 48b illustrates a portion of an exemplary embodiment of pseudocode for performing an SPC method.

FIG. 49 illustrates an exemplary embodiment of pseudocode for a Spike Sorting technique.

FIG. 50 illustrates an exemplary embodiment of pseudocode for bit encoding techniques.

FIG. 51a illustrates a portion of an exemplary embodiment of code for bit encoding techniques.

FIG. 51b illustrates a portion of an exemplary embodiment of code for bit encoding techniques.

FIG. 52 illustrates an exemplary embodiment of pseudocode for a Startup Procedure.

FIG. 53 illustrates an exemplary embodiment of pseudocode for a Provisioning Procedure.

FIG. 54 illustrates an exemplary embodiment of pseudocode for a Configuration Interface.

FIG. 55 illustrates an exemplary embodiment of pseudocode for a Stimulation Interface.

FIG. 56 illustrates an exemplary embodiment of pseudocode for a Recording Interface.

FIG. 57 illustrates an exemplary embodiment of pseudocode for a Status Interface.

FIG. 58 illustrates an exemplary embodiment of pseudocode for a temperature and power monitoring module.

FIG. 59 illustrates an exemplary embodiment of pseudocode for a Startup Procedure.

FIG. 60 illustrates an exemplary embodiment of pseudocode for a Provisioning Procedure.

FIG. 61a illustrates a portion of an exemplary embodiment of pseudocode for a command execution procedure.

FIG. 61b illustrates a portion of an exemplary embodiment of pseudocode for a command execution procedure.

FIG. 61c illustrates a portion of an exemplary embodiment of pseudocode for a command execution procedure.

FIG. 62 illustrates an exemplary embodiment of pseudocode for a data streaming procedure.

FIG. 63 illustrates an exemplary block diagram of a Gateway.

FIG. 64 illustrates an exemplary block diagram of the Cloud.

FIG. 65 illustrates an exemplary embodiment of pseudocode for a command message.

FIG. 66 illustrates an exemplary embodiment of pseudocode for a Configuration Command.

FIG. 67 illustrates an exemplary embodiment of pseudocode for a Stimulation Command.

FIG. 68 illustrates an exemplary embodiment of pseudocode for an Activation Command.

FIG. 69 illustrates an exemplary embodiment of pseudocode for an OTA Command.

FIG. 70 illustrates an exemplary embodiment of pseudocode for a Recording Control Command.

FIG. 71 illustrates an exemplary embodiment of pseudocode for a Status Command.

FIG. 72 illustrates an exemplary embodiment of pseudocode for a command message.

FIG. 73 illustrates an exemplary embodiment of pseudocode for a command message.

FIG. 74 illustrates an exemplary embodiment of pseudocode for a data message.

FIG. 75 illustrates an exemplary block diagram of an architecture for data ingestion and data processing.

FIG. 76 illustrates an exemplary embodiment of pseudocode for an API that may be used to specify the input for real time processing.

FIG. 77 illustrates an exemplary embodiment of pseudocode for an API that may be used to specify the pre-processing for real time processing.

FIG. 78 illustrates an exemplary embodiment of pseudocode for an API that may be used to specify the machine learning processing for real time processing.

FIG. 79a illustrates a portion of an exemplary embodiment of pseudocode for an API that may be used to specify the output for real time processing.

FIG. 79b illustrates a portion of an exemplary embodiment of pseudocode for an API that may be used to specify the output for real time processing.

FIG. 80 illustrates an exemplary embodiment of pseudocode for an API that may be used to specify the input for batch processing.

FIG. 81 illustrates an exemplary embodiment of pseudocode for an API that may be used to specify the machine learning for training new models for batch processing.

FIG. 82 illustrates an exemplary embodiment of pseudocode for an API that may be used to specify custom blocks for batch processing.

FIG. 83 illustrates an exemplary embodiment of pseudocode for an API that may be used for output from batch processing.

FIG. 84 illustrates an exemplary block diagram of an automatic pipeline.

FIG. 85 illustrates an exemplary embodiment of a module for autonomous processes.

FIG. 86 illustrates an exemplary embodiment of a cascading module for workflows.

FIG. 87 illustrates an exemplary embodiment of a pipeline for processing.

FIG. 88 illustrates an exemplary embodiment of a Machine Learning (ML) Toolbox.

FIG. 89 illustrates an exemplary embodiment of a pipeline for processing.

FIG. 90 illustrates an exemplary embodiment of a portion of a process of fabrication of CNT implant devices.

FIG. 91 illustrates an exemplary embodiment of a portion of a process of fabrication of CNT implant devices.

FIG. 92 illustrates an exemplary embodiment of a recording and stimulation signal and data flow on an implant device.

FIG. 93 illustrates an exemplary embodiment of a recording and stimulation signal and data flow on the Gateway and Cloud.

FIG. 94 illustrates an exemplary block diagram of an embodiment of an implant device electrical system.

FIG. 95 illustrates an exemplary embodiment of a portion of an implant device electrical system.

FIG. 96 illustrates an exemplary embodiment of a portion of an implant device electrode connection and firing distribution.

FIG. 97 illustrates an exemplary embodiment of a portion of triggering of the first ADC and the quantization of the action potential.

FIG. 98 illustrates an exemplary block diagram of multiplexer connections.

FIG. 99 illustrates an exemplary block diagram of a Gain Block.

FIG. 100 illustrates an exemplary block diagram of a Gain Block.

FIG. 101 illustrates an exemplary block diagram of an ADC.

FIG. 102 illustrates an exemplary block diagram of a DAC Block.

FIG. 103 illustrates an example of light scattering effects with wavelength.

FIG. 104 illustrates an exemplary block diagram of a computing device in which embodiments of the present systems and method may be implemented.

FIG. 105 is an exemplary block diagram of a system, according to embodiments of the present systems and methods.

FIG. 106 is an exemplary representation of the brain areas and associated functions.

FIG. 107 is an exemplary block diagram of a Closed Loop Control System that may be used by embodiments of the present systems and methods.

FIGS. 108a-d are an exemplary block diagram of an overall architecture of a system, according to embodiments of the present systems and methods.

FIG. 109 is an exemplary pseudocode diagram of a search process, according to embodiments of the present systems and methods.

FIG. 110 is an exemplary block diagram of a computer system, according to embodiments of the present systems and methods.

FIG. 111 is an exemplary block diagram of a cloud computing system, according to embodiments of the present systems and methods.

FIGS. 112a-c are an exemplary block diagram of an Orchestrator architecture, according to embodiments of the present systems and methods.

FIG. 113 is an exemplary illustration of processing workflow of a Selector Component, according to embodiments of the present systems and methods.

FIG. 114 is an exemplary representation of a family of genetic algorithms, according to embodiments of the present systems and methods.

FIG. 115 is an exemplary illustration of a genetic algorithm applied to digit strings, according to embodiments of the present systems and methods.

FIG. 116 is an exemplary illustration of a genetic algorithm, according to embodiments of the present systems and methods.

FIG. 117 shows exemplary flow diagrams of genetic algorithms, according to embodiments of the present systems and methods.

FIG. 118 is an exemplary illustration of Bayesian networks, according to embodiments of the present systems and methods.

FIG. 119 is an exemplary flow diagram of a process of constructing a Bayesian network, according to embodiments of the present systems and methods.

FIG. 120 is an exemplary pseudocode diagram of an Enumeration-Ask process, according to embodiments of the present systems and methods.

FIG. 121 is an exemplary pseudocode diagram of an Elimination-Ask process, according to embodiments of the present systems and methods.

FIG. 122 is an exemplary pseudocode diagram of a Likelihood Weighting process, according to embodiments of the present systems and methods.

FIG. 123 is an exemplary pseudocode flow diagram of a Gibbs Sampling process, according to embodiments of the present systems and methods.

FIG. 124 is an exemplary block diagram of a Critic-selector mechanism on personality layer, according to embodiments of the present systems and methods.

FIG. 125 is an exemplary block diagram of Data ingestion and data processing, according to embodiments of the present systems and methods.

FIG. 126 is an exemplary block diagram of a computer system, in which processes involved in the embodiments described herein may be implemented.

FIG. 127 is an exemplary illustration of an FCU/MCP device.

FIG. 128 is an exemplary illustration of coprocessor functions for implementing the manipulation of cellular structures via signaling.

FIG. 129 is an exemplary illustration of an embodiment of an apparatus in which the present techniques may be implemented.

FIG. 130 is an exemplary illustration of an embodiment of hardware implementation of the read and write modality hierarchy.

FIG. 131 is an exemplary illustration of an embodiment of the read/write modality usage in the detection and treatment of a neurological disorder, such as Alzheimer's disease.

FIG. 132 is an exemplary illustration of a higher-level view of the relationship between sensors, or read modality elements, and effectors, or write modality elements.

FIG. 133 is an exemplary illustration of an embodiment of the translation of neural code, from neurotransmitter and spike/pulse sequences, to action potentials, to frequency oscillations, and finally to cognitive output including speech and behavior.

FIG. 134 is an exemplary illustration of an embodiment of a schematic of the multiple levels at which the FCU analyzer operates.

FIG. 135 is a flow diagram of the process of autofluorescence.

FIG. 136 is an exemplary illustration of a flow diagram of an FCU-based mechanism for exchanging information within the brain: endogenous photon-triggered neuropsin transduction.

FIG. 137 is an exemplary illustration of an embodiment of an apparatus in which the present techniques may be implemented.

FIG. 138 is an exemplary illustration of photonic transduction in NAH Oxidase (NOX) and NAD(P)H.

FIG. 139 is an exemplary block diagram of a system 13900 that utilizes the FCU.

FIG. 140 is an exemplary block diagram of system architecture of a dopamine sensor system.

FIG. 141 is an exemplary illustration of the generation and transmission of neural signals in the nervous system.

FIG. 142 is an exemplary illustration of the waveform of neural spikes, according to neurological experiments.

FIG. 143 is an exemplary illustration of a simulation of the neural spike in MATLAB.

FIG. 144 is an exemplary illustration of the results of an experiment on spike frequency modulation (SFM).

FIG. 145 is an exemplary illustration of the mechanism of dSFM transforming a sequence of spikes into analog activation via motor neuron.

FIG. 146 is an exemplary illustration of the externally detected neural signals resulting from dSFM in brain-machine interface.

### DETAILED DESCRIPTION

The following patent applications are incorporated herein in their entirety: U.S. patent application Ser. No. 15/257,019, filed Sep. 6, 2016, U.S. patent application Ser. No. 15/431,283, filed Feb. 13, 2017, U.S. patent application Ser. No. 15/431,550, filed Feb. 13, 2017, U.S. patent application Ser. No. 15/458,179, filed Mar. 14, 2017, U.S. patent application Ser. No. 15/495,959, U.S. Provisional App. No. 62/214,443, filed Sep. 4, 2015, U.S. Provisional App. No. 62/294,435, filed Feb. 12, 2016, U.S. Provisional App. No. 62/294,485, filed Feb. 12, 2016, U.S. Provisional App. No. 62/308,212, filed Mar. 14, 2016, U.S. Provisional App. No. 62/326,007, filed April 104, 2016, U.S. Provisional App. No. 62/353,343, filed June 104, 2016, U.S. Provisional App. No. 62/397,474, filed September 104, 2016, U.S. Provisional App. No. 62/510,498, filed May 24, 2017, U.S. Provisional App. No. 62/510,519, filed May 24, 2017, U.S. Provisional App. No. 62/511,532, filed May 26, 2017, U.S. Provisional App. No. 62/515,133, filed Jun. 5, 2017, U.S. Provisional App. No. 62/534,671, filed Jul. 19, 2017, U.S. Provisional App. No. 62/560,750, filed Sep. 20, 2017, U.S. Provisional App. No. 62/588,210, filed Nov. 17, 2017, U.S. Provisional App. No. 62/658,764, filed Apr. 17, 2018, and U.S. Provisional App. No. 62/665,611, filed May 2, 2018.

Embodiments of the present invention may provide techniques for brain interfacing, mapping neuronal structure (Google earth for brains), manipulating cellular structure, cognitive, and brain augmentation via implants, and curing, not just managing, neurological disorders.

Embodiments may include a Brain Computer Interface (BCI), such as non-invasive BCI, including techniques such as EEG-based biofeedbacks (DREEM, MUSE, etc.), Transcranial Magnetic Stimulation (TMS), Magnetic Resonance Imaging (MRI), Transcutaneous Electrical Nerve Stimulation (TENS), etc., and invasive BCI, including techniques such as Nerve Cuffs (sacral, vagus, etc.), Cortical stimulation (flexible, electrodes), Spinal implants, etc. Functional neurosurgery may be performed using techniques such as Deep Brain Stimulation (DBS), which may modulate peak expiratory flow rates and disrupt cycles causing tremors and seizures. DBS may be effective for many neurological conditions—even depression. DBS may be able to control many systems in the body, such as cardiac function (neural pacemaker) and urological function (midbrain DBS) DBS is invasive but highly effective for many conditions. New approaches to neurosurgery may be, for example, 1,000-10,000 times more accurate than existing methods and 100 times less expensive than existing methods and may include techniques such as Optical Optogenetics triggering individual neurons and Lower level techniques such as direct modulation of endogenous photonic network, optical modulation of Neuropsin and the NAD(P)H cycle, etc. In embodiments, the Fundamental Code Unit (FCU) of the Brain, or Brain Code may enable intelligent, interactive modulation.

The successful development of new interventions for neurological disorders requires first and foremost, a strong theoretical framework for understanding healthy brain function and the brain's capacity for intelligent action. Such a theoretical framework is shown in FIG. 1 and may include a multi-level model of information exchange in biological systems, and an understanding, from language to cognitive concepts, down to the synaptic, molecular, and atomic interactions that guide brain development and function. These processes are closely inter-related and can be described mathematically in a uniform manner.

Embodiments may utilize the Fundamental Code Unit (FCU) of the Brain, or Brain Code. An exemplary block diagram of the Brain Code 200 is shown in FIG. 2. The example shown in FIG. 2 illustrates the decoding use of this language by mapping higher-order cognitive and behavioral processes to observed neurological states. For example, healthy vs diseased functions and tissues may be mapped, as a lack of function indicates circuits that may be diseased. The Brain Code is biological, not human. FCU is the most fundamental cognitive unit, analogous to quantum units, like photons, gravitons, etc., and the letters of the Brain Code are analogous to DNA codes such as A, G, C, T. Neurophysiological processes map to higher order function and are expressed differently at different levels of cognition. Such processes have the same underlying mathematical properties (unitary) and may map function from absence of function (NDD). FCU may be expressed as read modalities or write modalities. The Brain Code is further described below. A Unary Mathematical Framework of The Fundamental Code Unit is as follows:

Begin with a set S (uncountably infinite) representing brain regions which may be activated by some means. Introduce a σ-algebra A on this set, and call the elements a∈A activation sets (by definition a⊂S). Now introduce a second set W whose elements are labeled concepts in the brain which correspond to words. For some subset [symbol] ⊂A there is a mapping P: a′∈ [symbol] w∈W called the concept activation mapping. The element a′ of [symbol] arc action potentials. Let \tilde{P}: w∈W [symbol] ã∈ [symbol] be a mapping called the brain activation mapping. Let μ be a measure on S, and let [symbol] : A→{+, −} be a parity mapping. An axiology is a mapping Ξ: W→+, −} generated by computing

f(w)=∫ₐ [symbol] (s)dμ

with

a=\tilde{P}(w)

and then projecting

Ξ(w)=sign(f),

wherein:

| Symbol | Description | Properties |
|---|---|---|
| S | Brain regions | |
| A | Activation sets | α ∈ A ⇒ α ∈ S |
| [symbol] | Concept activation sets | [symbol] ⊂ A |
| W | Concepts | |
| P | Concept activation mapping | P: α' ∈ [symbol] w ∈ W |
| Ξ | Axiology | Ξ: W → {+, −} |
| [symbol] | Parity mapping | |
| μ | Weight mapping | |

Big Data in healthcare may include stethoscope data, MRI data, MEG data, EKG data, EEG data, PET data, etc., medical device data, implant data, wearable collected data 24×7, smartphones collected data—audio, video, motion, game/response, cloud data—now have unlimited storage and processing. Neurology has particularly Big Data as the brain is most complex system in the known universe. Such data may include full brain imaging, scans, modeling and may provide new mapping capabilities—optogenetics can now probe to determine functional circuits.

Multimodal Analysis and Diagnostics may be provided by using the Fundamental Code Unit (FCU) and Brain Code (BC). FCU provides the mathematical framework for meaningfully combining all data, built upon foundational neurophysiological processes (quantum Fibonacci 5:3), FCU maps neurophysiological processes to higher order brain function and language. FCU provides the means for decoding the language of the brain. Advanced AI and deep/extreme machine learning may be used . . . AI+IA=AC. Multimodal analysis provides the most accurate detection. Multimodal analysis can see through comorbidities and multitasking that make traditional detection difficult. Multimodal analysis can detect (and quantify) many formerly undiagnosable diseases (AD, PD, PTSD, etc.) and can diagnose conditions earlier than other methods (critical for NDDs).

Impairments of motor and none motor control are linked with factors related to the severity of the neurodegenerative disease therefore represent a potential domain space to detect PD. There is evidence that suggests that global cognitive changes are reflected in detectable changes in speech, therefore speech impairments may be possible markers of the onset and progression of Parkinson's disease. By coding and analyzing the meta characteristics of human speech and muscle movement, it may be possible to identify patterns associated with varying levels of cognitive functioning. Using a novel analysis framework that integrates multiple data streams, this research has sought to characterize the earliest deviations from normal neurocognitive functioning in NDG patients.

Embodiments may include two main functional/structural elements—the BrainOS Engine and the KIWI implantable neural sensor and stimulation device. The BrainOS, described further below, may include functional elements such as a Deep Cognitive Neural Network (DCNN) and a solution Architecture, as described below. The Deep Cognitive Neural Network (DCNN) architecture may integrate both convolutional feedforward and recurrent network principles, and may employ a novel queuing theory driven design to create perception and reasoning characteristics similar to the human brain.

Embodiments may provide gene expression profiling of single cells in the brain, whose contents may be extracted, for example, using a robotic probe. In order to convert these circuit-level targets into molecular targets, so as to look for drugs that bind to these molecular targets, the robotic probe may take a small piece of hollow glass and extract mRNA and other molecules from the cell to provide a molecular characterization of the cell type. The molecules that uniquely define a cell may serve as novel drug targets, and provide handles for further investigations of those cells. Embodiments may provide an ultraprecise platform for neural prosthetics. For example, for some brain disorders, such as those in which a large quantity of neurons is lost, a drug therapy may not be powerful enough to augment the remaining circuits. Embodiments may directly enter information into the brain in order to repair brain computations that have gone awry. Embodiments may include molecular methods, and hardware for stimulating the brain with light (described below), for better control of neural circuits that have gone awry in the brain. For example, embodiments may quiet down an epileptic seizure or repair blindness. Embodiments may further include whole-brain recording, to enable closed-loop processing—record info from the brain, compute what needs to be provided to the brain, and then transmit that information to the brain.

Embodiments may provide new approaches to pharmaceuticals. For example, photosynthetic molecules (optogenetics) may enable single neurons to be switched on and off. Implantable wireless 3D arrays of optical elements may be used in animals to identify functional circuits, replicate neural damage, and reverse-engineer repairs. From circuit-level targets to molecular targets molecules can uniquely identify a cell, which may be the target. A robotic probe may extract mRNA from molecules in the cell. Such molecular characterization may enable novel drug targeting. Gene expression from single cells in the brain may be automatically extracted and identified by the robotic probe.

Embodiments may utilize a BrainOS AI Engine, an example of which is shown in FIG. 3. BrainOS AI Engine may provide a comprehensive AI system capable of capturing data from different input sources, performing data enhancement using a variety of neural network architectures and generating, fine-tuning, validating, and combining to create powerful ensembles of models. Embodiments may provide functionality such as Contextual Awareness, Sentiment Analysis, Situational Awareness, Multi-modal Analysis, Orchestrator/Qualifier, Intent Based Learning, Infrastructure Management, etc. This may provide advantages such as Broader Application, Better Accuracy, Lower Resource Consumption, Quicker Learning, and Training, etc. Further, the BrainOS AI Engine may provide benefits. For example, the Deep Cognitive Neural Network (DCNN) architecture enables highly energy efficient computing with remarkably fast decision making and excellent generalization (long-term learning), and significantly outperforms Multi-Layer Perceptron (MLP) neural structures. As the volume and complexity of available data grows, the computational inefficiency of MLP solutions will generate an unsustainable need for hardware expansions, and processing latencies detrimental to critical, time sensitive activities. As the volume and complexity of available data grows, the computational inefficiency of MLP solutions will generate an unsustainable need for hardware expansions, and processing latencies detrimental to critical, time sensitive activities.

Examples of BrainOS Use Cases are shown in FIG. 4. The core features of the BrainOS are flexibility and scalability. The system can be adapted for a large array of existing problems, and extended with new approaches. An example of a BrainOS Architecture is shown in FIG. 5. An example of a Wellness Use Case is shown in FIG. 6.

The traditional belief is that the brain is electrochemical through regional ionization, action potentials, etc. However, such mechanisms are too slow, too hot, and use too much energy to be responsible for all neural function. Rather, there are also optical circuits in the brain. For example, at a level underlying neural firing, photons are utilized, such as in the neuropsin-controlled, cGMP-mediated transduction cascade cycle shown in FIG. 7. Neuropsin is bistable, with two states—(a) and (b). Neuropsin(a) plus a 380 nm photon yields Neuropsin(b), while Neuropsin(b) plus a 470 nm photon yields Neuropsin(a) and G-protein activation. A self-regulating optical cycle in the neocortex has been identified, which is active during periods of increased neural spiking activity. This cycle is linked to both increased neural activity and to neuroplastic changes such as memory formation in the hippocampus.

This optical mechanism not only explains the famous “Energy Paradox of the Brain”, but also enables entirely new methods of optical neurosurgery. The role bistable Neuropsin has in the activation of neuroplasticity-associated signaling pathways within the synaptic cleft creates many potential uses in computing: Neuropsin could serve as a transistor for organic biochip architecture. Such biochips could be grown from cells from patients and be self-powered. An entire neurophotonic system could serve as the core components for nanoscale optical computer and may enables entirely new methods of optical neurosurgery.

The KIWI, described further below, may provide an interface with neural tissue. Embodiments may record more neurons than previously possible. Embodiments may interpret recorded signals in real-time to formulate responses. Embodiments may electrically stimulate (write/modulate) neurons in real time. Embodiments may provide real-time, full data capture and cloud-based analysis. Embodiments may decode the Language of the Brain. Embodiments may utilize Carbon Nanotubes CNTs to increase points of neural connection and improve bio acceptance. Embodiments may utilize the FCU mathematical foundation, Brain Code theory, and Intention Awareness theory. Embodiments may include a 3D probe design, a closed-loop architecture, wireless communication and power, device and cloud integration, Collaborative research initiative (API/SDK), and deep machine learning. In embodiments, the device may carry and deliver immunological therapy, pharmacological agents, and stem cell treatments with great precision. Further, subsequent recordings may serve as a uniquely specific measure of treatment efficacy/progress in existing therapies.

An example of a KIWI system 800 is shown in FIG. 8. In this example, KIWI system 800 includes a Sensor Module 806—a small device that uses carbon nanotube (CNT) electrodes to make neural connection, an Electronics Platform 804—connected to the sensor module 800 via a cable and residing under the skull, and an External Interrogator 802, which will provide power and communications to implanted components and will be worn on the head.

In embodiments, system 800 may include an electronics platform 804 and a small (for example, <Icc) sensor module 806, connected by a miniaturized cable providing power and communication between these two units. Sensor module 806 may provide the interface and signal conditioning to the CNT array, and the electronics platform may house the processors, communication, and power management hardware. Power may be provided wirelessly by a head-mounted interrogator 802, which may also include a high-speed wireless data interface for communicating to the implant. The implant may operate completely under wireless power, removing the need for an implanted battery.

The electronics platform 804 may be designed to be placed between the skull and dura matter, allowing for the most efficient transfer of wireless power. High-speed wireless communication operating at a peak data rate of, for example, 4 Gb/s, will allow for maximum power efficiency, since the required throughput of the system is less than 5% of the wireless system capacity. This allows the wireless system to spend over 95% of its time in sleep mode, minimizing power consumption. The electronics platform 804 may include a low-power processor coupled with a programmable accelerator for DSP workloads. This ultra-low power compute system may run the spike sorting algorithms and manage the wireless communication. The electronics platform 804 may contain the electronics needed to receive the data from the sensor module, store it temporarily, and then forward it out on the platform's radio. The electronics platform 804 may be integrated using flexible PCBs into the appropriate medically-accepted housing and feedthrough connections. The electronics platform 804 may include integration of the charging and telemetry antennas into the miniaturized bio compatible package. In embodiments, the electronics platform 804 will NOT require a battery. Thus the system will work when the External Interrogator 802 is in place; removing the External Interrogator 802 depowers the system, rendering it inert. This is an important safety consideration when implementing autonomous feedback within the brain.

In embodiments, sensor module 806 may include an integrated front end System-On-Chip to provide pre-amplification and multiplexing of detected signals, as well as stimulus for outgoing neural signals, all contained in a volume of less than, for example, 1 cc. Covering the surface of the sensor will be, for example, 10,000 fibers made of, for example, carbon nanotube network filaments. These fibers may be built on an interfacial substrate and surrounded by a gel within a dissolvable membrane, such as Dextrane, Gelatine, or Collicoat. The gel coating will attract neurons to the implant, while the exposed CNT surface will provide excellent neuron attachment. This will further reduce the risk of damaging sensitive surface tissue during surgery and minimize adverse tissue reactions following implant insertion, protecting both the patient and the electrodes. The sensor module will be able to sense signals from pyramidal layers III down to layer VI of any brain cortex region.

In embodiments, electronics platform 804 may include the electronics needed to receive the data from the sensor module, store it temporarily, and then forward it out on the platform's radio. The electronics assembly may be integrated using flexible PCBs into the appropriate medically-accepted housing and feedthrough connections. The electronics 804 platform may include integration of the charging and telemetry antennas into the miniaturized bio compatible package. In embodiments, electronics platform 804 may not require a battery. Thus the system will work when the interrogator is in place; removing the interrogator depowers the system, rendering it inert. This is an important safety consideration when implementing autonomous feedback within the brain.

In embodiments, control and configuration of the platform may be performed from external interrogator 802 and for streaming data to the external interrogator Control and configuration data sent to the electronics platform 804 requires reliable delivery, but only limited throughput is required. However, the streaming data from the electronics platform 804 to the external interrogator 802 requires significant data throughput, making issues related to latency requirements important considerations. In embodiments, the wireless communication may be designed to support, for example, 10,000 channels or more, depending on the data size and sampling frequency of each channel. For example, if channels are sampled at a 1 kHz sampling rate and use a 12-bit analog-to-digital converter (ADC), then each channel requires a throughput of 12 kb/s. If there are 1000 channels, then the total streaming throughput to the Interrogator is 12 Mb/s. If there are 10,000 channels then the required throughput is 120 Mb/s. These data rates cannot be provided with a low data rate system like Bluetooth. However, Wi-Fi chips may be used to provide this high-speed data transfer. The electronics platform 804 may use an IEEE 802.11ac chip that supports up to 80 MHz bandwidth in the 5 GHz frequency band. This device has a peak data rate of 390 Mb/s.

The external interrogator 802 may use similar chips to the internal electronics platform 804, however, external interrogator 802 may include the software necessary for it to operate as a Wi-Fi access point (AP), while the internal electronics platform 804 may operate as a Wi-Fi station (STA). The external interrogator 802 may support two antennas for receive diversity so as to provide excellent signal-to-noise ratio (SNR) even if the interrogator is rotated on the skull and not perfectly aligned with the internal electronics platform. This may provide robust performance and ensures that the high throughput is available even under less than ideal laboratory conditions. Control and configuration of the platform may be provided from the external interrogator 802 and for streaming data to the external interrogator 802. Control and configuration data sent to the electronics platform 804 requires reliable delivery, only limited throughput is required. However, the streaming data from the electronics platform 804 to the external interrogator 802 requires significant data throughput, making issues related to latency requirements important considerations. The wireless communication may be designed to support 10,000 channels or more, depending on the data size and sampling frequency of each channel.

Fundamental Code Unit (FCU) algorithms may provide extremely high rates of data compression (>90%), association and throughput, enabling the KIWI to transcribe neural signals in high volume. A cloud platform may be used to harbor the parallel data flow and FCU analytic engine powered by neurocomputational algorithms and deep machine learning. KIWI data may be uploaded to the cloud wirelessly from the interrogator. A suite of algorithms may analyze and formulate instructions for electrical neuromodulations in a closed loop feedback system. Integrated stimulation/control, recording/readout, and modulated stimulation parameters may allow simultaneous electrical recording and stimulation.

Embodiments may provide decoding of the language of the brain and may be used in healthy patients to enhance natural human capabilities, as well as preemptive treatment for disorders/diseases. For example, the KIWI system 800 may be used, alone or in combination with other read modalities, to capture electrical and optical signals from electrophysiological neural signals of brain tissue, encode the captured electrical and optical signals using the Fundamental Code Unit, an input the encoded signals to the BrainOS. The BrainOS may then automatically generate one or more machine learning models that model the behavior of the neural tissue. Such models may then be used to generate signals, which may be encoded using the Fundamental Code Unit. The generated signals may then be applied to the neural tissue using the KIWI system 800, alone or in combination with other write modalities, to provide electrophysiological stimulation of the brain tissue.

In embodiments, a carbon nanotube (CNT) based electrode array may serve as a building block enabling high-density neural connections in a manner that is non-destructive to tissue. These electrodes may be integrated with solid-state imager readout circuitry (ROIC). For example, modern imager ROIC devices may have pixel densities on a micron pitch scale, which may be configured for single neuron voltage readout. Likewise, CNT electrodes and LED diodes (for optical stimulation) may be heterogeneously integrated on single a ROIC that could both optically stimulate and read the electrical potential from individual neurons.

In embodiments, a large number of electrically active brain-probing sites may be provided, along with long-term use. In embodiments, an implantable neural connecting probing system may be enabled by compliant, biocompatible, carbon nanotube (CNT) electrical wires. In embodiments, these contacts may directly stimulate and readout a high density of individual neural signals using read-out integrated circuit technology (ROIC) similar to that employed in focal plane arrays used in imaging applications.

In embodiments, an ROIC may include a large array of “pixels”, each consisting of a photodiode, and small signal amplifier. In embodiments, the photodiode may be processed as a light emitting diode, and the input to the amplifier may be provided by the CNT connection to the neuron. In this manner, neurons may be stimulated optically, and interrogated electrically. In embodiments, CNT electrical connection to neural tissue may be provided. In embodiments, a small pitch (2-20 micron) CNT array may be compatible with ROIC designs.

An exemplary embodiment of a Biological Co-Processor System (BCP) 900 is shown in FIG. 9. In embodiments, BCP 900 may include a neuromodulatory system comprising one, two, or more inductively-recharged neural implants 902 (the implant device), two earbuds 906, which may include wireless and various sensors, together known as the Brain Code Collection System (BCCS) 910. These devices may work independently, but together may form a closed-loop system that provides the BCP 900 with bidirectional guidance of both internal (neural) and external (behavioral and physiological) conditions. The BCCS earbuds 906 may read the brain for oscillatory rhythms from internal onboard EEG and analyze their co-modulation across frequency bands, spike-phase correlations, spike population dynamics, and other patterns derived from data received from the implant devices 902, correlating internal and external behaviors. The BCP may further comprise Gateway 911, which may include computing devices, such as a smartphone, personal computer, tablet computer, etc., and cloud computing services, such as the Fundamental Code Unit (FCU) 912 cloud computing services, which is a mathematical framework that enables the various BCCS 910 sensor feeds and implant device 902 neural impulses to be rapidly and meaningfully combined.

The FCU 912 may provide common temporal and spatial coordinates for the BCP 900 and resides in all components of the system (implants, earbuds, app, cloud) ensuring consistent mapping across different data types and devices. FCU 912 algorithms may provide extremely high rates of data compression, association, and throughput, enabling the implant device 902 to transcribe neural signals in high volume. Each implant device 902 may have an embedded AI processor, optical neurostimulation capabilities, and electrical recording capabilities. The implant device 902 may consist of two types of microfabricated carbon nanotube (CNT) neural interfaces, a processor unit for radio transmission and I/O, a light modulation and detection silicon photonic chip, an inductive coil for remote power transfer and an independent receiver system, where the signal processing may reside. The BCP 900 system may comprise four components: (1) the implant device 902 implant(s), (2) the BCCS 910 and (3) the cloud services (with API and SDK) and (4) an inductive power supply.

The implant device, an example of which is shown in FIG. 10, may be an ultra-low power computing device with interconnects that can attach to nerve and/or brain tissue and read signals/voltages and/or stimulate those tissues with electrical or optical pulses. This multi-physics interaction between the implant device and the tissue may be performed through two back-to-back arrays of optic fibers coated with single wall carbon nanotubes (CNTs). The CNTs may be chosen due to their structure, which has been shown to readily attach to tissue and also due to their remarkable electrical properties. Effectively, the CNTs may serve as electrochemical and optical sensors and measurement/stimulation electrodes. The device may be implanted in the brain or other parts of the body to attach to the nervous system, although this document focuses on attaching to the brain to treat neurological disorders. The implant device may include a communication module to transmit data to a Gateway device such as cell phone or other nearby computer which can in turn analyze data, give input to the implant device, and/or send the data to the Cloud for deep analysis.

The implant device may provide a revolutionary brain-computer interface for research in Neuroscience and medicine, being a closed-loop neural modulator informed by internal and external conditions. The possible therapeutic applications are numerous. For example, the implant device could be used for treatment of chronic pain, spinal cord injury, stroke, sensory deficits, and neurological disorders such as epilepsy, Parkinson's, Alzheimer's, and PTSD, all of which have evidence supporting the efficacy of neurostimulation therapy.

Turning briefly to FIG. 10, each implant device 902 implant may be, for example, an oblate spheroid (for example, 0.98×0.97×1.0 cm), a design inspired by the radial characteristics of an implant device 902 fruit. In the center of the implant is a nucleus surrounded by a fleshy membrane. The nucleus may house the processing, transmitting, and receiving circuitry 1008, including an embedded processor for local preprocessing, read and write instructions, the modulation scheme, and an optical FPGA dedicated for real time optical modulation. It may also contain a CMOS dedicated integrated front-end circuit developed for a pre-amplification and multiplexing of the neural signals recorded, 4G-MM for offline storage, wireless transceiver, inductive power receiver, and an optical modulation unit. Covering the nucleus are, for example, 1 million fibers 1002 made of single walled carbon nanotubes (SWCNT) and, for example, 1100 geometrically distributed optical fibers coated with SWCNT, connected in the same manner as the SWCNT fibers, wrapping around a central primary processing nucleus. Fibers may be built on a flexible interface substrate and surrounded by a gel/flesh membrane. When implanted, the membrane casing will slowly dissolve, naturally exposing the probes to a cellular environment with limited risk of rejection. For example, the gel may be relatively solid at about 25° C. and liquid at about 37° C. The lubrication of the CNT probes will attract neurons to the implant. The implant device 902 implant will be able to record from pyramidal layers II-III down to layer VI of any brain cortex region. Also shown in FIG. 10 are delay line devices 1004, light sources, such as vertical-cavity surface-emitting lasers 1006 (VCSELs), and antenna1010.

Returning to FIG. 9, the BCCS earbud 906, also shown in FIG. 11, wirelessly communicates with the implant device 902. The earbud contains a signal amplifier and a relay for modulation schemes, algorithms, and instructions to and from the implant. The BCCS earbud 906 also has additional functions, such as EEG and vestibular sensors, which will serve as crosscheck metrics to measure efficacy and provide global behavioral, physiological, and cognitive data along with neural data on the same timescale.

A cloud platform 912, also shown in FIG. 12, may include the parallel data flow and FCU 912 analytic engine powered by neuro-computational algorithms and extreme machine learning. EEG, ECG, and other physiological data (external and internal) will be uploaded to the cloud wirelessly from the BCCS 910 and implant device 902. A suite of algorithms will analyze the aggregate datastream and formulate instructions for optimal electrical and/or optical neuromodulations in a closed loop feedback system. Integrated stimulation/control, recording/readout, and modulated stimulation parameters will allow simultaneous optical and/or electrical recording and stimulation.

An inductive powering system 914, also shown in FIG. 13, may be used recharge the implant device 902 implant (see FIG. 9). Various wearable and/or kinetic inductive power technologies may be utilized during the design phase, including a retainer/mouthguard, a head-mounted cap to be worn at night, or an under the pillow charging mat.

Combined electro and optogenetic approach enables precise (ON/OFF) control of specific target neurons and circuits. Unary controls in combination with rapid closed loop controls in the implant device's microchip will enable neural synapse firings with intensity, and frequency modulation.

Integrating SWCNT nanotechnology with optical fibers enables both optogenetic writing and electrical neurostimulation capabilities.

CNTs are biologically compatible, enabling the implant device to be stably implanted for long periods of time.

A dissolvable membrane, such as Dextrane, Gelatine, or Collicoat, will limit the risk of damaging sensitive surface tissue during surgery and minimize adverse tissue reactions following the implant insertion trauma. This will protect both the patient and the CNTs.

The implant device will be in the brain parenchyma, rather than tethering the implant to the skull, which can be a major contributor to adverse tissue reactions.

The implant device's open hardware architecture can record data from all pyramidal layers II-III down to layer VI offering several advantages in terms of data quality.

Closed loop architecture enables dynamic, informed response based on live internal and external conditions.

Big data approach utilizing smartphone apps, SDKs, and websites/APIs will provide visual, aggregate, and actionable real-time biofeedback and software modification capabilities.

Big data approach utilizing cloud API will provide storage to capture extremely large volumes of data. The cloud platform also provides the massive processing power required to analyze these huge data sets across subject profiles and a plurality of research databases (PPMI, PDRS, etc.).

Open software architecture SDK will allow the creation of new applications and different protocols for clinical and research use, by partners, researchers, and third parties.

The BCCS will be able to synchronously capture EEG, ECG, PulseOx, QT intervals, BP, HR, RR, true body temperature, body posture, movement, skin conductance, vestibular data, and audio data to provide a rich set of multimodal data streams to dynamically correlate internal states read by the implant device and external states observed by the BCCS, a process which will help to effectively map neural pathways and function.

A passive inductive power unit and the BCCS earbud amplifier will be used external to the cranium, allowing the implant device to be small, low power and of low energy consumption. Any design for an extended-use implant without such an external component would need to be considerably larger (and of a finite lifespan).

The BCP data flow (internal and external) allows machine learning, prior experience, and real time biofeedback to autonomously guide implant device neuromodulation. Eventually the BCP will achieve an advanced level of sensitivity and will be able to autonomously sense neuron activity and guide light and/or electrical stimulation as needed.

Autonomous stimulation will be guided by intuitive algorithms and operational self-monitoring during awake state and sleep. Personal profiles and personalized signatures of neural activity will be learned and coded over time.

The BCP system takes two distinct but complementary approaches: a direct approach by means of recording brain activity and an indirect approach deduced from the multimodal aggregate analysis of peripheral effectors such as temperature, cardiac activity, body posture and motion, sensory testing etc. This simultaneous and coupled analysis of the interplay between the brain “activities and functions” (including physiological, chemical and behavioral activities) and its peripheral effectors and the influence of the effectors on the brain “activities and functions” has never been done before.

Simultaneous brain recording and stimulation of the same region allows us to take account of the initial state of the neurons and their environment, enabling comprehension of the neurons properties and network as well as brain functions (as the data are only valid for the specific conditions in which they were obtained). Methods which are forced to ignore this initial state have limited potential for understanding the full system.

Implant device Development—in an embodiment, an approach to solving density challenges combines traditional photolithographic thin-film techniques with origami design elements to increase density and adaptability of neuronal interfaces. Compared to traditional metal or glass electrodes, polymers such as CNT are flexible, strong, extremely thin, highly biocompatible, highly conductive, and have low contact impedance, which permits bidirectional interfacing with the brain (Vitale et al., 2015). These properties are especially valuable for the construction of high-density electrode arrays designed for chronic and/or long-term use in the brain. Our approach to precision and accuracy supersedes the current state of the art (SOA), which is limited to only being able to fit certain regions of the brain. These limits are due both to the physical design of the interface inserted and also to the limits of tethered communication within deeper cortical areas. The implant device, on the other hand, is wireless and inductively powered, and so is implantable anywhere in the brain with a subdural transceiver, to allow reading of neurons both at the surface and in 3D. CNT fibers will allow for bidirectional input and output. CNTs will also enable more biocompatible, longer-lasting designs—current neural implants work well for short periods of time, but chronic or long-term use of neural electrodes has been difficult to achieve. The main reasons for this are: 1) degradation of the electrode, 2) using oversized electrodes to attain sufficient signal-to-noise ratio during recording, and 3) the body's natural immune response to implantation. Although there is a strong desire among neurologists to record chronic neural activity, electrodes used today can damage brain tissue and lose their electrical contacts over time (McConnell et al., 2009, Prasad et al., 2012). This is of particular concern in the case of deep cortical implants, so alternative materials, design principles, and insertion techniques are needed. CNT is a biocompatible material that has been studied for long-term use in the brain.

Optogenetics may be used to facilitate selective, high-speed neuronal activation. Optogenetics pairs light-sensitive genes with a light source to selectively switch brain cells on or off. Some embodiments may mostly deliver light to one spot, whereas brain activity usually involves complex sequences of activation in different locations. Other embodiments may take optogenetics into three dimensions, with the ability to send patterns of light to neurons at various coordinates in the brain. For example, embodiments may include a technology in which light-sensitive ion channels are expressed in target neurons allowing their activity to be controlled by light. By coating optical fibers (˜8 μm) with dense, thin (˜1 μm) CNT conformal coatings, optical modulation units may be built within the nucleus of the implant device that can deliver light to precise locations deep within the brain while recording electrical activity at the same target locations. The light-activated proteins channelrhodopsin-2 and halorhodopsin may be used to activate and inhibit neurons in response to light of different wavelengths. Precisely-targetable fiber arrays and in vivo-optimized expression systems may enable the use of this tool in awake, behaving primates.

Such embodiments may not only solve the famous “Energy Paradox of the Brain”, but may also enables entirely new methods of optical neurosurgery. Further, the role bistable Neuropsin has in the activation of neuroplasticity-associated signaling pathways within the synaptic cleft may create many potential uses in computing. For example, neuropsin could serve as a transistor for organic biochip architecture, biochips could be grown from cells from patient and self-powered, and entire neurophotonic system could serve as the core components for nanoscale optical computers.

A suite of brain to digital and digital to brain (B2D:D2B) algorithms may be used for transducing neuron output into digital information. These algorithms may be theoretically-grounded computational models corresponding to the theory of similarity computation in Bottom-Up and Top-Down signal interaction. These neurally-derived algorithms may use mathematical abstractions of the representations, transformations, and learning rules employed by the brain, which will correspond to the models derived from the data and correspond to the general dynamic logic and mathematical framework, account for uncertainty in the data, as well as provide predictive analytical capabilities for events yet to take place. The BCP analytics may provide advantages over conventional systems in similarity estimation, generalization from a single exemplar, and recognition of more than one class of stimuli within a complex composition (“scene”) given single exemplars from each class. This enables the system to generalize and abstract non-sensory data (EEG, speech, movement). Combined, these provide both global (brain-wide) and fine detail (for example, communication between and within cytoarchitectonic areas) modalities for reading and writing across different timescales.

The implant device may be a microfabricated carbon nanotube neural implant that may provide, for example, reading from ≥1,000,000 neurons, writing to ≥100,000 neurons, and reading and writing simultaneously to ≥1,000 neurons. The BCCS may include multisensory wireless inductive earbuds and behavioral sensors and provide wireless communication with implant device, inductively recharge implant device, provide Bluetooth communication with a secure app on smartphones, tablets, etc., and may provide interfacing with cloud—API, SDK and secure website for clinicians, patients (users)

The implant device and BCCS devices may be used in combination with FCU, BC and IA algorithms to translate audial cortex output, matching internal and external stimulus (for example, output) to transcribe thought into human readable text.

The BCP may provide advantages over conventional systems by providing a closed loop neural interface system that uses big data analytics and extreme machine learning on a secure cloud platform, to read from and intelligently respond to the brain using both electrical and optical modulation. The FCU unary framework enables extremely high-speed compression, encryption, and abstract data representation, allowing the system to process multimodal and multi-device data in real-time. This capability is of great interest and benefit to both cognitive neurosciences and basic comprehension of brain function and dysfunction because: (1) it combines high dynamic spatiotemporal and functional resolution with the ability to show how the brain responds to demands made by change in the environment and adapts over time through its multiple relationships of brain-behavior and brain-effectors; (2) it assesses causality because the data streams are exhibited temporally relative to the initial state and each state thereafter by integrating physiological and behavioral factors such as global synchrony, attention level, fatigues etc., and (3) data collection does not affect, interfere or disrupt any function during the process.

The BCP may provide advantages over conventional systems by recording from all six layers of the primary A1 cortex and simultaneously from the mPFC, with very high spatial resolution along the axis of the penetrating probe by combining CNT with fiber optic probes that wrap around a central nucleus. By including the principal input layer IV and the intra columnar projection layers, as well as the major output layers V and VI, brain activity can be monitored with unprecedented resolution. The recording array will be combined with optogenetic stimulation fibers, which are considerably larger and stiffer than electrode arrays. CNT fibers will be used as recording electrodes at an unprecedented scale and within a highly dense geometry.

Carbon nanotubes address the most important challenges that currently limit the long-term use of neural electrodes and their unique combination of electrical, mechanical and nanoscale properties make them particularly attractive for use in neural implants. CNTs allow for the use of smaller electrodes by reducing impedance, improving signal-to-noise ratios while improving the biological response to neural electrodes. Measurements show that the output photocurrent varies linearly with the input light intensity and can be modulated by bias-voltage. The quantum efficiency of CNTs are about 0.063% in 760 Torr ambient, and becomes 1.93% in 3 mTorr ambient. A SWCNT fiber bundle can be stably implanted in the brain for long periods of time and attract neurons to grow or self-attaching to the probes. CNT and optical fibers will be an excellent shank to wrap a polymer array around.

Returning to FIG. 10, the optical fibers 1002 will be coated with SWCNTs and make electrical connections with the underlying delay line. The delay line 1004 will be transparent to allow light from the vertical-cavity surface-emitting lasers 1006 (VCSELs) to reach the optical fibers. The delay lines 1004 potentially make the electrical signal position-dependent by comparing the time between pulses measured at the outputs. Provided the pulses are of sufficient intensity and individual pulses are sufficiently separated in time (>1 μs or so), the difference between pulse arrival times could be related to the position on the array. Combining this with spatially controlled optical excitation (i.e., by turning on specific VCSELs 1006) would further help to quantify position, as VCSEL pulses excite a small region at the end of the adjacent fiber. These pulses are measured at a position on the delay line close to this fiber, so if neighboring neurons fire, they are sensed by nearby fibers (i.e., the SWCNTs on the fibers) and would generate additional pulses that could then be tracked over time with the delay line, mapping out the path. The SWCNT coated fiber array 1002 would be randomly connected to the underlying VCSEL array as we will not have control over the fiber locations in the bundle. The substrate connectors will be graphitic nano joints to a single-walled carbon nanotube, we will also utilize the IBM CNT connect technique for other connectors.

Carbon nanotubes are ideal for integration into a neural interface and the technical feasibility of doing so is well documented. The use of CNT allows for one unit to function as recording electrodes and stimulating optical fibers. The optical transceivers will be integrated as a separate die on a silicon substrate, tightly-coupled to logic dice (a.k.a. “2.5D integration”). The choice of materials reflects the positive results of recent studies demonstrating the impact of flexibility and density of implanted probes on CNNI tissue responses. CNTs are not only biocompatible in robust coatings, but they are supportive to neuron growth and adhesion. It has been found that CNTs actually promote neurite growth, neuronal adhesion, and viability of cultured neurons under traditional conditions. The nanoscale dimensions of the CNT allow for molecular interactions with neurons and the nanoscale surface topography is ideal for attracting neurons. In fact, they have been shown to improve network formation between neighboring neurons by the presence of increased spontaneous postsynaptic currents, which is a widely accepted way to judge health of network structure. Additionally, functionalization of CNT can be used to alter neuron behavior significantly. In terms of the brain's immune response, CNT have been shown to decrease the negative impact of the implanted electrodes. Upon injury to neuronal tissue, microglia (the macrophage-like cells of the nervous system) respond to protect the neurons from the foreign body and heal the injury, and astrocytes change morphology and begin to secrete glial fibrillary acidic protein to form the glial scar. This scar encapsulates the electrode and separates it from the neurons. However, carbon nanomaterials have been shown to decrease the number and function of astrocytes in the brain, which in turn decreases the glial scar formation.

Optogenetic tools may be used to enable precise silencing of specific target neurons. Using unary controls in combinations and in rapid closed loop controls within the implant device will enable neural synapse firings with highly precise timing, intensity, and frequency modulation. Optical neuromodulation has many benefits over traditional electrode-based neurostimulation. This strategy will allow precision stimulation in near real time.

The implant device uses a 3D design (and dissoluble membrane), both of which may provide advantages over conventional systems. The dissoluble membrane protects both the patient and the implant during surgery and the lubricant and contraction encourages neural encroachment and adherence to CNTs upon dissolution. This design maximizes neural connectivity and adhesion, while minimizing implant size. Implant device size is further reduced through inductive charging.

The BCP system aims at producing a significant leap in neuroscience research not only in scale but also in precision. The method of optical reading and writing at the same time, using SWCNT optrodes, can be combined with current cell marking techniques to guide electrodes and optic fibers to specific regions of the brain. One of the biggest challenges facing neuroscientists is to know for certain if they are hitting the right spot when performing in vivo experiments, whether it is an electrophysiological recording or an optogenetic stimulation. Cell marking techniques, on the other hand, have made a lot of progress during the past 20 years with the use of new viral approaches as well as Cre-Lox recombination techniques to express cell markers in specific sites of the brain. This has allowed, for example, the expression of fluorescent Calcium indicators in target locations without affecting surrounding regions, which is commonly used in in vivo Calcium imaging. Our technique of simultaneous optical reading and writing makes it possible to insert optrodes and guide them through brain tissue until they “sense” optical changes corresponding to the activity of target cells that express a Calcium indicator. This will reduce, to a great extent, the probability of off-target recordings and stimulations.

The synchronous connection between the implant device and BCCS will likely lead to rapid advances in understanding the key circuits and language of the brain. The BCP provides researchers with a more thorough (and contextual) understanding of neural signaling patterns than ever before, enabling far more responsive brain-machine interfaces (for example, enabling a paralyzed patient to control a computer, quadcopter or mechanical prosthetic). A wireless implanted device might allow a PD patient to not only quell tremors but actually regain motor capacity, even just minutes after receiving an implant. By combining these technologies with behavioral and physiological metrics, we hope to open up new horizons for the analysis of cognition. Our multimodal diagnostic and analysis allows for an approach of analyzing brain machinery at higher data resolution. The data method could be considered a first step in progressing medicine from snapshots of macro anatomo-physiology to continuous, in-vivo monitoring of micro anatomo-physiology. The in-vivo study of a brain's parcel may give us a real-time relationship of the different components and their functionality, from which the complex functional mechanism of the brain machinery could be highlighted. Giving rise to new medical approaches of diagnosis, treatment, and research. If the animal experiences of two implants prove efficacy and lack of any harm to animal or humans, the BCP may allow us to define a powerful new technique for brain-functional mapping which could be used to systematically analyze and understand the interconnectivity of each brain region, along with the functionality of each region.

Therapeutic aims may include use of the device as a brain stimulator, and indirect by data from recordings highlighting the mechanism(s) by which several diseases occur, owing to implant device's ability to record a basic global neuronal state of a brain region and the dynamic neuronal interplay. The modifications which occur during its normal activity enable us to understand the neuronal properties and the function of a given brain region. Our device is able to give us the dynamic continuum of the whole activity of the considered region and thus provide important insights into the fundamental mechanisms underlying both normal brain function and abnormal brain functions (for example, brain disease). The potential for these findings to be translated into therapies are endless because this device may be used in any region of the brain and represents the first synthesis of a closed-loop neural modulator informed by internal and external conditions. The BCP provides a large amount of information and could be used to explore any brain disease within a real dynamic, in vivo condition. If successful, the potential of this device for the diagnosis of organic brain diseases is enormous and it could be an important complement to MRI for the diagnosis of non-organic disease. The possible therapeutic use of this device may also include chronic pain, tinnitus, and epilepsy. The device could be used in focal epileptic zone owing to its optogenetic capacity to control excitability of a specific populations of neurons. Even if the device does not cure epilepsy, it may help to control otherwise refractory seizures and help to avoid surgery. Nonetheless optimizing the place of this device in therapy for epilepsy will require further study and clinical experience.

Recent demonstrations of direct, real-time interfaces between living brain tissue and artificial devices, such as with computer cursors, robots and mechanical prostheses, have opened new avenues for experimental and clinical investigation of Brain Machine Interfaces (BMIs). BMIs have rapidly become incorporated into the development of ‘neuroprosthetics,’ which are devices that use neurophysiological signals from undamaged components of the central or peripheral nervous system to allow patients to regain motor capabilities. Indeed, several findings already point to a bright future for neuroprosthetics in many domains of rehabilitation medicine. For example, scalp electroencephalography (EEG) signals linked to a computer have provided ‘locked-in’ patients with a channel of communication. BMI technology, based on multi-electrode single-unit recordings, a technique originally introduced in rodents and later demonstrated in non-human primates, has yet to be transferred to clinical neuroprosthetics. Human trials in which paralyzed patients were chronically implanted with cone electrodes or intracortical multi-electrode arrays allowed the direct control of computer cursors. However, these trials also raised a number of issues that need to be addressed before the true clinical worth of invasive BMIs can be realized. These include the reliability, safety and biocompatibility of chronic brain implants and the longevity of chronic recordings, areas that require greater attention if BMIs are to be safely moved into the clinical arena. In addition to offering hope for a potential future therapy for the rehabilitation of severely paralyzed patients, BMIs can be extremely useful platforms to test various ideas for how populations of neurons encode information in behaving animals. Together with other methods, research on BMIs has contributed to the growing consensus that distributed neural ensembles, rather than the single neuron, constitute the true functional unit of the CNS responsible for the production of a wide behavioral repertoire (reference).

When designing an interface between a living tissue and an electronic device, there are important factors to consider. Particularly, the structural and chemical differences between these two systems; the electrode ability to transfer charge; and the temporal-spatial resolution of recording and stimulation. Traditional multi-electrode array (MEAs) for neuronal applications present several limitations: low signal to noise ratio (SNR), low spatial resolution (leading to poor site specificity) and limited biocompatibility (easily encapsulated with non-conductive undesirable glial scar tissue) which increases tissue injury and immune response. Neural electrodes should also accommodate for differences in mechanical properties, bioactivity, and mechanisms of charge transport, to ensure both the viability of the cells and the effectiveness of the electrical interface. An ideal material to meet these requirements is carbon nanotubes (CNTs). CNTs are well suited for neural electrical interfacing applications owing to their large surface area, superior electrical and mechanical properties, and the ability to support excellent neuronal cell adhesion. Over the past several years it has been demonstrated as a promising material for neural interfacing applications. It was shown that the CNTs coating enhanced both recording and electrical stimulation of neurons in culture, rats, and monkeys by decreasing the electrode impedance and increasing charge transfer. Related work demonstrated the single-walled CNTs composite can serve as material foundation of neural electrodes with chemical structure better adapted with long-term integration with the neural tissue, which was tested on rabbit retinas, crayfish in vitro, and rat cortex in vivo.

Using long CNTs implanted into the brain has many advantages, for instance an optical fiber with CNTs protruding from it, but this technology has not been trialed in vivo or expanded to very large numbers of recording channels. Characterization in vitro showed that the tissue contact impedance of CNT fibers was lower than that of state-of-the-art metal electrodes, chronic studies in vivo in parkinsonian rodents also showed that CNT fiber microelectrodes stimulated neurons as effectively as metal electrodes. Stimulation of hippocampal neurons in vitro with vertically multiwalled CNTs electrodes suggested CNTs were capable of providing far safer and efficacious solutions for neural prostheses than metal electrode approaches. CNT-MEA chips proved useful for in vitro studies of stem cell differentiation, drug screening, and toxicity, synaptic plasticity, and pathogenic processes involved in epilepsy, stroke, and neurodegenerative diseases. Nanotubes are a great feature for reducing adverse tissue reactions and maximizing the chances of high-quality recordings, but squeezing a lot of hardware into a small volume of tissue will likely produce severe astroglial reactions and neuronal death. At the same time, CNTs could extend the recording capabilities of the implant beyond the astroglial scar, without increasing the foreign body response and the magnitude of tissue reactions. Implantation of traditional, rigid silicon electrode arrays has been shown to produce a progressive breakdown of the blood-brain barrier and recruitment of an astroglial scar with an associated microglia response.

Neural implant geometry and design is highly dependent on animal model used, where larger animals will see a somewhat less dramatic deterioration in recording quality and quantity, so early trials in rats probably shouldn't be too focused on obtaining very long-term recordings on a very large number of channels. While loss of yield due to abiotic failures is a manufacturing process and handling problem, biotic failures driven hostile tissue reactions can only be addressed by implementing design concepts shown to reduce reactive astrogliosis, microglial recruitment and neuronal death (Prasad, A. et al., 2012; McGonnell, G C. et al., 2009).

Conventional thin film probes can fit hundreds of leads into one penetrating shank. Rolling up a planar design would come with several benefits: first, it would decrease the amount of tissue damage a wide 2D-structure would produce. This is essential for the very high densities we are aiming for. Second, it would stiffen the probe, making it easier to penetrate tissue. Thirdly, a round cross section is preferable for reducing the foreign body response in the brain parenchyma. Finally, this design allows for potentially extremely dense architectures, as by combining several of these probes into a 10×10 array of 1 cm², an implant using this technology could potentially deploy several tens of thousands of leads in a multielectrode array, and could be conceivably combined with optical fibers for stimulation within an electronic-photonic microarray implant. A design of an implantable electrode system may be a 3D electrode array attached to a platform on the cortical surface. Said platform would be used for signal processing and wireless communication.

Why coatings or composites with CNT? The unique combination of electrical, mechanical and nanoscale properties of carbon nanotubes (CNT) make them very attractive for use in NE. Recent CNT studies have tried different CNT coatings or composites on metal electrodes and growing full electrodes purely from CNT. Edward W. Keefer et al., (2008) was the first to do a recording study using different coatings made with CNT on electrodes. They found that CNT can help improve the electrode performance during recording by decreasing impedance, increasing charge transfer and increasing signal-to-noise ratio. CNT may improve the biological response to neural electrodes by minimizing risk of brain tissue rejection.

Why ICA for analysis? ICA signal separation is performed on a sample by sample basis where no information about spike shape is used. For this reason, it is possible to achieve good performance of sorting accuracy in terms of misses and false positives, especially in cases where the background noise is not stationary but fluctuate throughout trials, which is the fact based on biophysical and anatomical considerations but is ignored by most current spike sorting algorithms One assumption underlying this technique is that the unknown sources are independent, which is the case under the assumption that the extracellular space is electrically homogeneous, pairs of cells are less likely to be equidistant from both electrodes. The other assumption of this approach is that the number of channels must equal or greater than the number of sources, which can yield advantages for large-scaled recordings.

Exemplary tables of advantages of aspects of technologies that may be utilized by embodiments are shown in FIGS. 14 and 15.

The two-implant device's may be implanted within the mPFC in addition to the A1 primary auditory cortex because this cortical area may be implicated in the pathogenesis of PTSD. Dopaminergic modulation of high-level cognition in Parkinson's disease and the role of the prefrontal cortex may be revealed by PET, as may widely distributed corticostriatal projections. The mPFC may also be implicated in psychiatric aspects of other disorders, for example deficits in executive functions, anxiety, and depression. By recording from the selected sensory areas and implanting two kiwis at same time, the chance of needing further surgical corrections may be reduced, and data recording may be increased. Knowledge may be extracted that may lead to corrections of associated cognitive deficit in conditions like PTSD but in general to cognitive decline as it occurs for many unknown indicators.

In an embodiment, the BCP hardware may be fabricated using electronic components available on the market today. In an embodiment, the implant device may be made with a microfabricated carbon nanotube (CNT) neural interface, a light modulation and detection silicon photonic chip, and an independent Central Processing Unit (CPU) where all the processing will preside. RF communication between the implant device and BCCS may carried out either by making use of the processor's Bluetooth capability or by implementing an independent RF transceiver in each of the two devices. The BCCS device may be calibrated to and securely integrated with the implant device. Exemplary block diagrams of embodiments of an implant device 1600 is shown in FIGS. 16 and 17, and are described further below.

As may be seen from FIG. 10, the implant device may be composed of two such hardware components in a back to back configuration, each one functioning independently. In embodiments, each of the two boards may be split into, for example, 100 tiles with 16 I/O pins. An exemplary embodiment of such a tile design is shown in FIG. 18. Each tile may include, for example, one Reference Pin 1802, five Ground Pins 1804, six Recording Pins 1806, and four pins for either Recording or Stimulation 1808. The specific function of each pin is described below. On one side the tile cells may be attached to CNTs, while on the other side, the tiles may interface with the hardware components needed to process the analog signals.

An exemplary embodiment of an arrangement of tiles is shown in FIG. 19. In this embodiment, the tiles may be physically arranged in a 10×10 matrix as shown. Each integrated circuit (application-specific integrated circuit (ASIC), field-programmable gate array (FPGA), etc.) may be connected to a tile block that is composed of, for example, 10×10 tiles. Thus, the integrated circuit may simultaneously read 10×10×10=1000 channels and simultaneously stimulate up to 10×10×4=400 channels. In an embodiment, the implant device may include two integrated circuits and be able to read up to 2000 channels and write up to 800 channels simultaneously.

Channel types that may be supported may include Optrodes and Electrodes. Optrodes (optical electrodes), may perform optical and electrical recording and stimulation. Optrodes may be composed of optical fiber coated with single walled carbon nanotubes. The optical fiber may be used for transporting light signals bidirectionally. Electrodes may perform only electrical recording and stimulation. The carbon nanotubes may be used to transport electric signals. In embodiments, the configuration may depend on the goals of the device implant for each individual patient. Thus, in embodiments, the implant device may support different configurations in terms of channels (number and type (electrical, optical, or chemical) of stimulation and/or recording channels) and Computing Power.

In embodiments, the power budget of the implant device may be in the range of about 100 μW to 1 mW. Embodiments of battery options, assuming an implant device autonomy of 72 hours may include:

Rechargeable Li-ion Battery: In embodiments, the battery may be as small as a grain of rice. The energy of such a battery would be only 3 mWh, or maybe less in normal operating conditions. If a more likely nominal capacity of 2 mWh is considered, this equates to a power budget of 30 μW over a period of 72 hours, in the case that a custom integrated circuit is not needed.

Rechargeable Silver Oxide Battery: In embodiments, a cylindrical Silver Oxide battery with a volume of about 30 cmm (cubic millimeters) may have a nominal capacity of 11 mWh. Over a period of 72 hours this equates to a power budget of about 160 μW. However, due to the chemistry of the Silver Oxide battery, it can only allow a limited number of recharge cycles.

Rechargeable Li—Po Battery: While expensive compared to the other two options, the Li—Po batteries promise about 1200 Wh/L, which would equate to 36 mWh for the same volume of 30 cmm. Over a period of 72 hours, this equates to a power budget of about 500 μW. Due to its high power density, the Li—Po battery has since long been used for pacemakers and may be used in embodiments of this application as well.

For safety reasons, the battery should not heat up more than 1° C. during charging.

Typical implant methods and medical implications. In the field of neural modulation, DBS surgery has been used for the symptomatic treatment of Parkinson's disease for a long time. The intervention implies the drilling of the skull and the insertion of the stimulation electrodes deep within the brain. After this step, another intervention inserts the pulse generator under the skin of the patient's chest, close to the collar bone. Severe intraoperative adverse events included vasovagal response, hypotension, and seizure. Postoperative imaging confirmed asymptomatic intracerebral hemorrhage (ICH), asymptomatic intraventricular hemorrhage, symptomatic ICH, and ischemic infarction, and was associated with hemiparesis and/or decreased consciousness. Long-term complications of DBS device implantation not requiring additional surgery included hardware discomfort and loss of desired effect in 10. Hardware-related complications requiring surgical revision included wound infections, lead malposition, and/or migration, component fracture, component malfunction, and loss of effect.

Under DARPA's Reliable Neural-Interface Technology (RE-NET) program, scientists have developed the stentrode, a chip that is far less invasive due to the fact that it is implanted to the brain through blood vessels without opening the skull. This approach was tested on sheep and the chip was inserted via a blood vessel in the neck and guided to the brain using real-time imaging. Once the chip reaches the target location it expands and attaches to the walls of the blood vessel to read the activity of the nearby neurons.

In embodiments, different implantation procedure may be used, and each has advantages and disadvantages.

Implant device Cyber Security. Billions of sensors that are already deployed lack protection against attacks that manipulate the physical properties of devices to cause sensors and embedded devices to malfunction. Analog signals such as sound or electromagnetic waves can be used as part of “transduction attacks” to spoof data by exploiting the physics of sensors.

A “return to classic engineering approaches” may be needed to cope with physics-based attacks on sensors and other embedded devices, including a focus on system-wide (versus component-specific) testing and the use of new manufacturing techniques to thwart certain types of transduction attacks.

Transduction attacks may target the physics of the hardware that underlies that software, including the circuit boards that discrete components are deployed on, or the materials that make up the components themselves. Although the attacks target vulnerabilities in the hardware, the consequences often arise in the software system, such as improper functioning or denial of service to a sensor or actuator. Hardware and software have what might be considered a “social contract” that analog information captured by sensors will be rendered faithfully as it is transformed into binary data that software can interpret and act on. But materials used to create sensors can be influenced by other phenomena—such as sound waves. Through the targeted use of such signals, the behavior of the sensor may be interfered with and even manipulated.

In embodiments, the implant device may take measures against vulnerability to accidental or malicious wave interferences.

Neuron Connection Interface. Due to their extraordinary properties, CNTs may be used in different roles, such as electrophysiological reading, electrophysiological stimulation, electrochemical detection, optical reading, and optical stimulation. Embodiments may include specialized implant devices that feature only one type of CNTs or hybrid implant devices with multiple types of CNTs, which may use artificial intelligence (AI) to manage them according to the nature of the application.

Carbon Nanotubes (CNTs) are a material with broad application, such as additives, polymers, and catalysts; in autoelectron emission, flat displays, gas discharge tubes, absorption, and screening of electromagnetic waves, energy conversion, lithium battery anodes, hydrogen storage, composite materials, nanoprobes, sensors, and supercapacitors. CNTs may be used as super-miniaturized chemical and biological sensors based on the fact that their voltage-current (V-I) curves change as a result of adsorption of specific molecules on their surface. Furthermore, the boundary (tip) of the CNT may be modified by functional groups, metal nanoparticles, polymers and metal oxides to increase the selectivity of the detectors built based on them, adding filtering capabilities to it.

CNTs have remarkable mechanical, thermal, and electrical properties. For example, the Young's modulus of CNTs, which is a measure of axial tensile stiffness, may be over 1 TPa (Aluminum has 70 GPa). CNTs may have a strength-to-weight ratio 500 times greater than Aluminum. The thermal conductivity of CNTs may be very high (approximately 3000 W/mK) in the axial direction and very small in the radial direction. CNTs may have a very high current carrying capacity and may have an electrical conductivity six orders of magnitude higher than copper. Due to their high mechanical and thermal stability and resistance to electromigration, CNTs may sustain current densities of up to 109 A/cm2. Depending on their chirality—the geometric orientation of the carbon atoms network—the electrical properties of the CNTs may change—they may behave either as conductors or semiconductors. In an electronic device this may allow both the active devices and interconnects to be made of CNTs.

In embodiments, CNTs may be used as Sensors, for functions such as Electrophysiological Recording, measuring the electrical potential in neural tissue by using CNTs as conductors, Electrochemical Recording, detecting neurotransmitters in neural tissue through fast-scan cyclic voltammetry (FSCV), Optical Recording, making CNTs sensitive to fluorescent substances by changing their chiral configuration, Neural Stimulators, Electrophysiological Stimulation, stimulating the brain neurons by using CNTs as conductors, Optical Stimulation, using Optogenetics techniques, and Electrochemical Stimulation.

Connection Method. When implant device is inserted in the brain, the CNTs may establish strong adhesive contact with the neuronal tissue, becoming able to measure the electrical field in their vicinity. The following approximate calculations provide an intuition on how the implant device CNTs will fit over the neural network. The brain cells may be in the range of 10-50 micrometers in diameter. The width of a CNT may be in the range of 0.7-50 nanometers. In embodiments, the optrodes (the CNT coated optic fibers) or electrodes (with CNT fiber) may be organized in 100 tiles arranged in a square configuration. Each tile may be made of a 4 by 4 array of optrodes. Therefore, the CNTs may be arranged in a 400×400 matrix. Given that one side of the KIWI optrode array may be about 1 cm, the interaxial distance between the CNTs is about 25 micrometers.

An exemplary illustration of an approximate representation of how the optrode array could fit over a dense neural network is shown in FIG. 20. In this example, the following assumptions have been made. The brain cells 2002 have been represented as circles 30 microns in diameter and 50 microns apart (distance between centers). The centers of the optrodes have been represented as squares 25 microns apart. The diameter of the CNT may be about 1000 times smaller than the diameter of the brain cell, so the CNTs would hardly be visible if they were drawn to scale. For better readability, an array of only 10 by 10 optrodes has been represented.

In order to obtain a clear reading from one single point of contact with the brain tissue and avoid electrical short circuit, it is important for the CNTs to remain upright and not stick to each other, which would naturally happen due to the force of molecular adhesion (van der Waals interactions). Soft lubricant gel may be used to ensure their upright position, as shown in FIG. 21. After the implant, due to its size, position, and optrodes configuration, the implant device may be able to connect to all neuron layers from I to VI, as shown in FIG. 22. At the other end, the CNTs 2302 may connect to the electrodes 2304 through which the neuron stimulation and reading will be performed, as shown in FIG. 23.

Electrophysiologic Detection of Voltage. In embodiments, CNTs may be used for deep brain recordings of voltages from neural tissues in their vicinities. For this task, CNT, based electrode arrays may be used that enable high-density neural connections in a manner that is non-destructive to the neuronal tissue. This method is feasible and efficient because of all the above-mentioned properties of CNTs—mechanical, thermal, and electrical.

Electrochemical Detection of Neurotransmitters. In embodiments, CNTs may be used in yarn macrostructures (which are several parallel CNTs) to detect neurotransmitters in vivo. Disk-shaped CNT yarns may detect electro-active transmitters, as shown in FIG. 24, which is a fast-scan cyclic voltammetry diagram of CNT yarn disk shaped (CNTy-D) microelectrodes and conventional microelectrodes detecting different neurotransmitter species. The method employed, fast-scan cyclic voltammetry (FSCV), is a technique by which changes in the extracellular concentration of electroactive molecules may be monitored when the electrode is ramped up to a certain threshold over time, and then it is ramped down to return to the initial potential.

Different surface structures (chirality) of the CNTs may result in different CV (Cyclic Voltage) responses towards each neurotransmitter species. The sensitivity of the CNT yarn microelectrodes may also be enhanced by different modification approaches: laser treatment may increase sensitivity towards dopamine, O₂ plasma etching may increase sensitivity towards dopamine, and anti-static gun treatment may increase surface area by increasing the roughness.

Fluorescent Carbon Nanotubes. The different geometries of the carbon atom network making up a CNT may determine different electronic properties. The different electronic properties may be correlated with different optical properties because their electronic band-gap between valence and conduction band may make the single walled CNTs fluorescent in the near infrared (NIR, 900-1600 nm). This property may enable the CNTs to be used for optical multiplexing because every chiral configuration could be used as a single color. An example of how carbon nanotube color changes with chiral index is shown in FIG. 25. The colors of the CNTs arise due to the absorption of light in the visible range. In this example, a sample with separated SWCNT of different chiralities and corresponding absorption and fluorescence spectra are shown, labelled with the main (n,m) chiral index component. Further, single walled CNTs used as optical sensors may exhibit a near Infrared emission range that coincides with the tissue transparency window.

The unique composition of the polymeric functionals used with single walled CNTs may enable them for the selective detection of neurotransmitters with high spatial resolution. For example, a fluorescent nanosensor array based on single-walled CNTs may be used for sensing dopamine from PC12 neuroprogenitor cells at high temporal (100 ms) and spatial (20.000 sensors per cell) resolution.

CNT arrays as a solution for spatially distributed current release. Techniques have been developed to map electrical microcircuits in the brain at far more detail than existing techniques, which are limited to tiny sections of the brain (or remain confined to simpler model organisms, like zebrafish).

In the brain, groups of neurons that connect up in microcircuits help us process information about things we see, smell, and taste. Knowing how many neurons and other types of cells make up these microcircuits would give scientists a deeper understanding of how the brain computes complex information.

Nanoengineered microelectrodes. Embodiments may use “nanoengineered electroporation microelectrodes” (NEMs). Electroporation is a microbiology technique that applies an electrical field to cells to increase the permeability (ease of penetration) of the cell membrane, allowing (in this case) fluorophores (fluorescent, or glowing dyes) to penetrate into the cells to label (identify parts of) the neural microcircuits (including the “inputs” and “outputs”) under a microscope. Such electrodes may be used to map out cells that make up a specific microcircuit in a part of a brain for a particular function. The electrodes may include a series of tiny pores (holes) near the end of a micropipette, produced using nano-engineering tools. The new design distributes the electrical current uniformly over a wider area (up to a radius of about 50 micrometers—the size of a typical neural microcircuit), with minimal cell damage. An example of an embodiment of a NEM can be seen in FIG. 26. By releasing the current through multiple openings, multiple neuron layers may be stimulated using the NEM. Multiple release points mean the current will be distributed in a wider area so that neurons will not suffer from a local current concentration (which one would create to stimulate a larger volume of tissue)

In embodiments, the configuration and implant position of the implant device may provide conditions for multi-point electric stimulation. With regards to reaching multiple layers of neurons, the implant device may connect to layers I to VI, due also to the length and geometrical configuration of the CNTs. With regards to the electrical potential distribution in the tissue, due to the 2000+CNT fibers populating it, the implant device may have a greater number of stimulation points, offering a superior spatial resolution.

Optical Fibers. In addition to embodiments of the implant device being able to read/write electric and electrochemical signals from/to the neurons through the CNTs, embodiments of the implant device may also have the capability of optically stimulating the neurons and reading optical signals from them. The optical interaction between the brain and the implant device may take place through an array of optical fibers in a process called optogenetics.

Optogenetics and fiber photometry are neuro-modulation technologies in neuroscience that utilizes a combination of light and genetics to control and monitor neurons in vivo. In embodiments, optogenetics and fiber photometry may provide the capability to map the amygdala, such as for fear conditioning, to perform studies for targeting pharmacotherapies and addiction via nucleus accumbens, for expression of pyramidal neurons in PFC, and for genetic components of social behavior and drug efficacy in neuropsychiatric disorders etc.

Optical Stimulation. Optogenetics is a technology in which light-sensitive ion channels may be virally expressed in target neurons allowing their activity to be controlled by light. By coating optical fibers with dense, thin CNT conformal coatings, embodiments may include optical modulation units within the nucleus of the implant device that may deliver light to precise locations deep within the brain, while recording electrical activity at the same target locations. As described below, the light-activated proteins Channelrhodopsin-2 and Halorhodopsin may be used to activate and inhibit neurons in response to light of different wavelengths and we are currently developing precisely targetable fiber arrays and in vivo-optimized expression systems to enable the use of this tools in awake, behaving primates.

The implant device software may be synchronized with optogenetic actuators and sensors and fiber photometry devices allowing for acquisition of behavioral data during experiments by using TTL (transistor-transistor logic) and a specially developed software interface. This brings research into a new realm with the possibility of simultaneous control of biochemical events of living freely behaving animals and the collection of this data in both high-throughput and real-time.

In order to be able to monitor and modulate the biochemical events in behaving animals, the animals must be able to move freely without being restricted by wires and tethers. Embodiments of the implant device may provide this capability due to the fact that all data exchanges and power delivery are wireless.

Embodiments of the implant device may be used for experiments mapping function of the amygdala such as fear conditioning, studies for targeting pharmacotherapies and addiction via nucleus accumbens, expression of pyramidal neurons in PFC and genetic components of social behavior and drug efficacy in neuropsychiatric disorders, etc. In embodiments, examples of optogenetic/fiber photometry systems that may be used may include SEIZURESCAN®, HOMECAGESCAN®, GROUPHOUSESCAN®, FREEZESCAN®, CHAMBERSCAN®, GAITSCAN®, TREADSCAN®, RUNWAYSCAN®, TOPSCAN®, AND SOCIALSCAN®.

Optical Sensing of Neurotransmitters. The optical sensing of neurotransmitters may have advantages over the electrochemical sensing techniques. For example, improved Lower limit of detection (the smallest substance concentration/quantity that can be detected), often reaching a nanomolar range or less (compared, for example, to 300 nM for dopamine detection using electrochemical sensing by CNT yarn microelectrodes. The broad range of optical spectrum may allow for the interference from other chemical species to be minimized. Optical sensing may provide high spatial resolution. The release and uptake of neurotransmitters may occur in a highly localized fashion; therefore the high spatial resolution refers to that fact that the sensors are small enough to identify which neurons are involved in these chemical interactions. Optical sensing may provide improved temporal resolution. The neurotransmitter release and uptake processes occur in a millisecond time range. Optical sensors may have a sampling rate that is high enough to detect the concentration changes.

Neuronal Data Recording. In embodiments, the implant device may include both optical fibers and CNTs that can have multiple roles. In such embodiments, the implant device may record neuronal activity data using, for example, any of the following three methods: Electrophysiological Recording, Optical Recording, and Electrochemical Recording. In embodiments, specialized implant devices may be used that feature only one type of neural interaction, hybrid implant devices may be used that feature all types of interaction. In the latter case, complex AI algorithms may be used for CNT management according to their properties.

The Electrophysiological Recording functionality relies on the special current carrying capacity of the CNTs. The Optical Recording may, for example, be performed in two ways. First, the implant device may use an on-board light-source to activate fluorescent cells and may use the dedicated optical fibers to record and transmit the data to the circuitry. Second, the fluorescent CNTs (polymer functionalized CNTs) may be used to optically identify the release of certain neurotransmitters.

The Electrochemical Recording functionality of the implant device may provide for the detection of released neurotransmitters based on analyzing the shape of the curve obtained by plotting current intensity over electric potential in fast-scan cyclic voltammetry.

Recording Capacities. In embodiments, the implant device may record up to 2,000 channels simultaneously. For example, such an embodiment may use the tile architecture described above (implant device Design), which includes 2 electrode/optrode boards, 10×10 tiles per board, and up to 10 recording channels per tile.

In embodiments, the reading and stimulation circuitry may be in the form of a readout-integrated circuit (ROIC), which may be similar to or a modification of, for example, a solid-state imaging array. The ROIC may include a large array of “pixels”, each consisting of a photodiode, and small signal amplifier. In embodiments, the photodiode may be processed as a light emitting diode, and the input to the amplifier may be provided by the CNT connection to the neuron. In this manner, neurons may be stimulated optically, and interrogated electrically. The ROIC may include CCD or CMOS photodiodes or other imaging cells, to receive optical signals, electrical receiving circuitry, to receive electrical signals, light outputting circuitry, such as LED or lasers, to output optical signals, and electrical transmitting circuitry, to transmit electrical signals.

Electrophysiological Recording. In electrophysiology—the oldest strategy for neural recording, an electrode is used to measure the local voltage at a recording site, which conveys information about the spiking activity of one or more nearby neurons. The number of recording sites may be smaller than the number of neurons recorded since each recording site may detect signals from multiple neurons in the area.

An example of an electrophysiological recording pipeline 2700 is shown in FIG. 27. Pipeline 2700 may include a plurality N of electrodes 2702, such as SWCNT fibers. The SWCNT fibers may each be connected to a preamplifier 2704, which may convert the weak electrical signal coming from the neurons into an output signal that is strong enough to be noise-tolerant and processing ready. The output signal from each preamplifier 2704 of a plurality N of preamplifiers 2704 may be input into an electrical Multiplexing Unit (MUX) 2706 having N inputs. Between the processing circuitry 2710 and MUX 2706 is a Select Line 2707, through which processing circuitry 2710 may communicate to MUX 2706 the channel to read through at that time. In order to be able to select from N inputs, the Select Line may specify log 2(N) bits, which means that it may contain that many connections. In an embodiment, there may be 1000 or more recording channels. In such an embodiment, it may be difficult to have a single Multiplexer that can switch among all of the inputs. Accordingly, in embodiments, the circuitry may include, for example, with two layers of multiplexers with 16 input channels each, as follows: 64 multiplexers connected to the CNTs, which feed into 4 multiplexers. In embodiments, there may be another layer of multiplexing as well. Embodiments may include any convenient arrangement of multiplexers to handle the number of recording channels.

From MUX 2706, the selected signal goes into Analog to Digital Converter (ADC) 2708, which converts the received analog value into a digital value, for example, 8, 10, or 12 bits, which is then passed along to processing circuitry 2710. Processing circuitry 2710 may include digital processing circuitry, such as one or more microprocessors, microcontrollers, digital signal processors (DSPs), custom or semi-custom circuitry, such as application specific integrated circuits (ASICs), field programmable circuitry, such as field programmable gate arrays (FPGAs), etc., or any other digital processing circuitry.

In order to minimize the interference between the recording and stimulation signals, in embodiments, the CNTs that are used for electrical recording may be used only for recording. Even so, given the proximity of all the CNTs, in embodiments, the recorded signal may be cleaned of the electric stimulation signal, which is may be much stronger than the signal input from the neurons.

Recording Formula. For calculating the recorded electrical voltage, embodiments may use the Ground that is closest to the Recording channel, and the Reference for negative values. Without the Reference, the negative values would be clipped to 0, and by this valuable information may be lost.

Optical Recording. In embodiments, the implant device may also record optically using optical properties of CNTs and/or optical fibers coated with CNTs. For Optical Recording, the neurons that have been modified, for example, genetically, to have fluorescent capabilities may be illuminated to trigger the fluorescence. The fluorescence may vary based on the voltage that is going through the membrane of the neuron. So, the recorded light intensities may correspond to the voltage strength of the neurons. In embodiments, the optic fiber in the optrode may be used for both optical stimulation and recording by way of a Beam Splitter, which may be positioned close to the optrode, to convert the two-way light circuit into two one-way light circuits.

An example of an embodiment of an optical recording pipeline 2800 is shown in FIG. 28. In this example, pipeline 2800 may include a plurality N of optrodes 2802, such as SWCNT coated optical fibers. The signal that comes from each optrode 2802 goes through a beam splitter 2804 into an Optical Modulator 2806, which may transform it from a baseband signal to a bandpass signal, that can be processed by the Optical processor 2810.

From the Optical Modulator 2806, the optical signal may be input to Optical Multiplexing Unit 2808, where based on the selection signal on select line 2812 from the Optical processor 2810, one channel may be selected to be read. The Select Line between Optical Multiplexing Unit 2808 and the Optical processor 2810 may, for example, be a digital electrical signal. The Optical processor 2810 may receive the selection instructions (which channel to read) from the processing circuitry 2814 over select line 2816.

The selected light signal from Optical Multiplexing Unit 2808 may be input to Optical processor 2810 through an optical connection. Optical processor 2810 may convert the light signal into a digital electrical signal, for example, 8, 10, or 12 bits, and outputs the digital signal to processing circuitry 2814. Processing circuitry 2814 may include digital processing circuitry, such as one or more microprocessors, microcontrollers, digital signal processors (DSPs), custom or semi-custom circuitry, such as application specific integrated circuits (ASICs), field programmable circuitry, such as field programmable gate arrays (FPGAs), etc., or any other digital processing circuitry.

An example of an embodiment of an optical recording pipeline 2900 is shown in FIG. 29. In this example, pipeline 2900 may include a plurality N of optrodes 2902, such as SWCNT coated optical fibers. The signal that comes from each optrode 2902 goes into Optical Multiplexing Unit 2904, where based on the selection signal on select line 2912 from processing circuitry 2914, one channel may be selected to be read. The Select Line between Optical Multiplexing Unit 2908 and the Optical processor 2910 may, for example, be a digital electrical signal.

The selected light signal from Optical Multiplexing Unit 2908 may be input to Photodiode 2906, which converts it into an analog electrical signal. This analog electrical signal may be passed than through a Signal Conditioning Unit 2908, which may perform filtering and amplification on the analog electrical signal. The processed analog electrical signal may then be input into Analog to Digital Converter (ADC) 2910, which may convert it into a digital electrical signal, for example, 8, 10, or 12 bits, and output the digital signal to processing circuitry 2814. Processing circuitry 2914 may include digital processing circuitry, such as one or more microprocessors, microcontrollers, digital signal processors (DSPs), custom or semi-custom circuitry, such as application specific integrated circuits (ASICs), field programmable circuitry, such as field programmable gate arrays (FPGAs), etc., or any other digital processing circuitry.

Electrochemical Recording. Although called Electrochemical Recording, in embodiments, this functionality may rely on the ability of embodiments to electrically stimulate the neural tissue (stimulation) and compute the current intensity (processing) by knowing the electrical resistivity. Electrochemical recording may be performed through the CNTs and may be based on the fast-scan cyclic-voltammetry (FSCV) technique to detect the neurotransmitters' release and uptake. The method involves subjecting neural tissue to an electric potential linearly increasing over time up to a certain threshold. After reaching the threshold, the electric potential is linearly ramped down to the initial value.

An example of a conceptual diagram of the cyclically applied potential is shown in FIG. 22.

The FSCV stimulation potential may be applied through a specific command given by the processing circuitry through the stimulation pipeline described below. The current at the working electrode is plotted versus the applied voltage to give the cyclic voltammogram trace. A few examples of how these cyclic voltammogram traces look are shown in FIG. 30. Therefore, the released neurotransmitters may be identified based on knowing the shape of their specific cyclic voltammogram trace.

Hybrid Recording: Justification and Specifics. Given that the Electrophysical Recording Pipeline may be built separately from the Optical Recording Pipeline, depending on the number of CNTs assigned to each one of the two methods, embodiments may be able to simultaneously record both electrophysically and optically. By combining both methods, embodiments may record more complex and novel insights about the functionality of the brain.

Pipeline Summary. An example of a high-level architecture 3100 of the pipelines presented above, as well as compression and data transmission to Gateway (Communication Platform) is shown in FIG. 31. The sense channels pipeline architecture highlights the components used for propagating the neurons recorded voltages to the Gateway component. As shown in this example, the architecture may include a plurality of sense channels 3102, zone selection/controller circuitry 3104, a plurality of recording pipelines 3106A-M, a plurality of data compression engines 3108A-M, and Parallel-In-Serial-Out Converter (PISO) 3110. Sense channels 3102, for example, electrical and/or optical sense channels including CNTs, SWCNTs, optical fibers, etc., may be input to zone selection/controller circuitry 3104. Zone selection/controller circuitry 3104 may select groups or zones of sense channels 3102 for input to recording pipelines 3106A-M. Recording pipelines 3106A-M may convert analog electrical and/or optical signals to digital electrical signals. Each recording pipeline 3106A-M may handle a plurality of sense channels 3102 and may include a plurality of instances of recording pipeline circuitry. For example, each instance of recording pipeline circuitry may include signal conditioning circuitry 3112, such amplifiers, filters, variable gain stages, etc., N to 1 analog MUX 3114, and ADC 3116. Each instance of recording pipeline circuitry may convert analog electrical and/or optical signals to digital electrical signals at a rate of 20 Kilo-samples per second (Ksps) per input sense channel 3102. Assuming, for this example, 10 bits per sample, each instance of recording pipeline circuitry may generate 200 Kilobits per second (Kbps) of data. As each analog MUX may multiplex N signals, ADC 3116 may generate 200N Kbps of data. The data from each recording pipeline 3106A-M may be input to a data compression engine 3108A-M, which may, for example, provide 100 times compression. Thus, in this example, each 200N Kbps data channel may be compressed to a 2N Kbps data channel. The outputs from each data compression engine 3108A-M may be input to PISO 3110, in which the M parallel 2N Kbps data channels may be serialized to form a single serial output data channel 3118, which may be input to processing circuitry (not shown). In this example, with 1000 sense channels 3102, serial output data channel 3118 may handle 2 Mega-bits per second (Mbps). The maximum sample rate and data rate may depend on the particular engineering design, such as the specifications of the processing circuitry, such as processor and memory.

Although in this example, ADC 3116 may provide 10-bit samples, any resolution ADC may be used. For example, ADCs with resolutions of 24 bits per sample are readily available. However, ADCs having less resolution may consume less power and may take up less space. Accordingly, ADCs having resolutions from 8 bits per sample to 12 bits per sample may provide a good tradeoff between resolution and power and space consumption. Likewise, ADCs having a variable number of bits per sample may be used. For example, such an ADC may provide a variable number of bits per sample of from 8 bits per sample to 12 bits per sample.

The measured data for each sense channel 3102 may represent the voltage from a small region of neural tissue. In embodiments, range of sample rates may be from about 1000 samples/second to about 20,000 samples/second. In embodiments, depending upon the number of sense channels 3102, the maximum compressed data generated throughput may be about 4 Mbps. In embodiments, data representing simultaneously recorded voltages may be grouped into data frames, where the number of recorded values encapsulated in one data frame may depend on the number of simultaneously active reading channels 3102, and on the transfer rate capabilities to the Gateway at that time. The recording process may adapt to the specific use case and the available transfer bandwidth to the Gateway using a recording rate and channel selection module. In embodiments, the same data sequential order within a frame may be maintained and the order of recordings in the frame may follow the physical distribution of the Recording Channels on the tile matrix. In embodiments, processing circuitry, such as input/output (I/O) Control circuitry and/or software may control and configure PISO 3118 and MUX 3114 capabilities.

Neural Activity Modulation. In embodiments, neural tissue may be stimulated using one or more of several techniques, such as Optical Stimulation (Optogenetics), Electrophysiological Stimulation, and Electrochemical Stimulation.

Optical Stimulation. Optogenetics is a method for brain stimulation/modulation by inducing well-defined neuronal events at a millisecond-time resolution, enabling optical control of the neural activity. The method may utilize physiological processes such as Channelrhodopsin-2 (ChR2): a light-sensitive ion channel, Halorhodopsin (NpHR): an optically activated chloride pump, and Archaerhodopsin (Arch): a proton pump. ChR2 and NpHR may be genetically expressed in neurons using a viral approach. Conventionally these viruses are injected in the neural tissue, but in embodiments, the virus vector may be carried on the tips of the CNTs. Due to their small dimensions, these viruses do not interfere with the reading and stimulation processes.

There are several types of Channelrhodopsins, each one responding to a particular wavelength. Some Channelrhodopsins stimulate neuronal activity (ChR2), while others inhibit it (NpHR). Therefore, the optical sensitivity of these proteins enables both the increasing/activation and decreasing/silencing of the voltage inside neurons, by targeted laser beams of blue and yellow light, respectively. The technique is deemed as safe, precise, and reversible.

Optogenetics may be used as a side-effect-free method for alleviating symptoms of neurological diseases which occur through either neuronal overexcitability, such as epilepsy, or underactivity, such as schizophrenia. One practical advantage is that optogenetics may have minimal instrumental interference with simultaneous electrophysiological techniques.

Examples of spike trains of ChR2 and NpHR expressing neurons when subjected to light beams of different wavelengths are shown in FIG. 32. FIG. 32, Ai shows an example of neuron expressing channelrhodopsin-2 fused to mCherry. FIG. 32, Aii shows an example of neuron expressing halorhodopsin fused to GFP. FIG. 32, Aiii shows an example of an overlay of Ai and Aii.

Optogenetics enable the optical control of individual neurons, but even neurons with no genetic modification have light sensitivity, such as in a circuit mediated by neuropsin (OPN5), a bistable photopigment, and driven by mitochondrial free radical production. This bistable circuit is a self-regulating cycle of photon-mediated events in the neocortex involving sequential interactions among 3 mitochondrial sources of endogenously-generated photons during periods of increased neural spiking activity: (a) near-UV photons (˜380 nm), a free radical reaction byproduct; (b) blue photons (˜470 nm) emitted by NAD(P)H upon absorption of near-UV photons; and (c) green photons (˜530 nm) generated by NAD(P)H oxidases, upon NAD(P)H-generated blue photon absorption. The bistable nature of this nanoscale quantum process provides evidence for an on/off (UNARY+/−) coding system existing at the most fundamental level of brain operation and provides a solid neurophysiological basis for the FCU. This phenomenon also provides an explanation for how the brain is able to process so much information with slower circuits and so little energy-quantum tunneling. Computers built from such material would be orders of magnitude faster than anything developed to date. The atomic scale of CNTs could potentially enable interfacing with this naturally optosensitive layer of the brain in the future, a system many orders of magnitude smaller than the neuron.

FIG. 33 illustrates an example of Poisson trains of spikes elicited by pulses of blue light (dashes), in two different neurons.

FIG. 34 illustrates an example of a light-driven spike blockade, demonstrated for (TOP) a representative hippocampal neuron, (BOTTOM) a population of 7 neurons. This example illustrates I-injection, neuronal firing induced by pulsed somatic current injection(300 pA, 4 ms). This example illustrates light, hyperpolarization induced by periods of yellow light (bars). This example illustrates I-injection+Light, yellow light drives Halo to block neuron spiking, leaving spikes elicited during periods of darkness intact.

FIG. 35 illustrates an example of (TOP) an action spectrum for ChR2 overlaid with absorption spectrum for N. pharaonis halorhodopsin and (BOTTOM) Hyperpolarization and depolarization events induced in a representative neuron by a Poisson train of alternating pulses (10 ms) of yellow and blue light.

FIG. 36 illustrates examples of the correlation between wavelengths (nm) and normalized cumulative charge for a number of different Channelrhodopsins expressing neurons. From all the Channelrhodopsins discovered types, Crimson red light stimulation is the most suited because in its case, the light intensity is proportional to how deep it travels in the brain.

In embodiments, the circuitry may be in the form of a readout-integrated circuit (ROIC), which may be similar to or a modification of, for example, a solid-state imaging array. The ROIC may include a large array of “pixels”, each consisting of a photodiode, and small signal amplifier. In embodiments, the photodiode may be processed as a light emitting diode, and the input to the amplifier may be provided by the CNT connection to the neuron. In this manner, neurons may be stimulated optically, and interrogated electrically. The ROIC may include CCD or CMOS photodiodes or other imaging cells, to receive optical signals, electrical receiving circuitry, to receive electrical signals, light outputting circuitry, such as LED or lasers, to output optical signals, and electrical transmitting circuitry, to transmit electrical signals.

An example of an embodiment of an optical stimulation pipeline 3700 is shown in FIG. 37. In this example, pipeline 3700 may include processing circuitry 3702. Processing circuitry 3702 may include digital processing circuitry, such as one or more microprocessors, microcontrollers, digital signal processors (DSPs), custom or semi-custom circuitry, such as application specific integrated circuits (ASICs), field programmable circuitry, such as field programmable gate arrays (FPGAs), etc., or any other digital processing circuitry.

Processing circuitry 3702 may encode stimulation commands for modulation of optical signal. For example, such commands may be 5 bits, for up to 32 different modulation commands. Processing circuitry 3702 may send one of the 32 possible commands and the data identifying the channel to be stimulated. Each command may be mapped into a wavelength and a light intensity, which may be encoded digitally and sent to optical processor 3704 on its digital in/out port, together with the channel on which the light may be transmitted.

Optical processor 3704 may transform the input digital electrical signal into an optical signal of the appropriate wavelength and intensity. Optical processor 3704 may then transmit the light signal to Optical Demultiplexing Unit (DEMUX) 3706, along with the desired channel on the Select Line 3714.

Optical Demultiplexing Unit 3706 may forward the light signal on the appropriate channel. Each light signal may pass through a Delay Line 3708 and then through an Optical Modulator 3710, which may adjust and amplify the signal to its appropriate values. The light signal then be transmitted through optrodes 3712, through the fibers, to the neurons.

An example of an embodiment of an optical stimulation pipeline 3800 is shown in FIG. 38. In this example, pipeline 3800 may include processing circuitry 3802. Processing circuitry 3802 may include digital processing circuitry, such as one or more microprocessors, microcontrollers, digital signal processors (DSPs), custom or semi-custom circuitry, such as application specific integrated circuits (ASICs), field programmable circuitry, such as field programmable gate arrays (FPGAs), etc., or any other digital processing circuitry.

Processing circuitry 3802 may encode stimulation commands for modulation of optical signal. For example, such commands may be 5 bits, for up to 32 different modulation commands. Processing circuitry 3802 may send one of the 32 possible commands and the data identifying the channel to be stimulated. Each command may be mapped into a wavelength and a light intensity, which may be encoded digitally and sent to DAC 3804, in which the digital electrical signal may be converted to an analog electrical signal.

The analog electrical signal may be amplified by a Signal Conditioning Unit 3806, to increase its amplitude to useful levels. From Signal Conditioning Unit 3806, the analog electrical signal may be input to an electrical Demultiplexing Unit (DEMUX) 3808. Based on the signal that comes from the processing circuitry 3802 on Select Line 3820, DEMUX 3808 may transmit the analog electrical signal on an appropriate channel to the LED 3810 that generates an optical signal of the required wavelength. LED 3810 may generate an optical signal, which may be transmitted through a Delay Line 3812, to an Optical Modulator 3814. From the Optical Modulator 3814, the optical signal may travel through an Optical Demultiplexing Unit, which, based on the received signal on select line 3822 from processing circuitry 3802, may forward the light beam to the correct optrode 3818.

In this exemplary embodiment, there are two demultiplexing units: an electric one 3808, which leads to the LED of the right wavelength, and an optical one 3816 which sends the light down the correct channel. Accordingly, embodiments may have as many light sources as wavelengths to be generated.

Electrophysiological Stimulation. Alzheimer's disease produces irreversible degradation to the brain to the point where there are not many treatment options. There are only a few medications available, which unfortunately cannot stop the symptoms from getting progressively worse or even fatal.

However, one potential treatment for diseases such as Alzheimer's may be deep brain stimulation. Deep brain stimulation works by continuously tickling neurons in the frontal lobe of the brain with electrodes. Patients who have these electrodes implanted may maintain more of their mental faculties than a group of control patients, who started out at similar stages of the disease.

Electrophysiology is a tool for deep brain stimulation in which electrical current is applied via electrodes implanted on/in the brain parenchyma. While optical stimulation is able to target specific neurons very precisely, electrical stimulation implies current dissipation in the surrounding area.

Electrophysiological Stimulation may be used for neuron stimulation by applying electrical current via CNTs that are connected to nanoelectrodes and are implanted directly in the brain parenchyma.

An example of an embodiment of an optical stimulation pipeline 3900 is shown in FIG. 39. In this example, pipeline 3900 may include processing circuitry 3902. Processing circuitry 3902 may include digital processing circuitry, such as one or more microprocessors, microcontrollers, digital signal processors (DSPs), custom or semi-custom circuitry, such as application specific integrated circuits (ASICs), field programmable circuitry, such as field programmable gate arrays (FPGAs), etc., or any other digital processing circuitry.

Processing circuitry 3902 may encode stimulation commands for the output signal. For example, such commands may be 5 bits, for up to 32 different modulation commands. Processing circuitry 3902 may send one of the 32 possible commands and the data identifying the channel to be stimulated. Each command may be mapped into a stimulation voltage, which may then be sent out from processing circuitry 3902 to Digital to Analog Converter (DAC) 3904, which converts the digital electrical signal to an analog electrical signal. The analog electrical signal may be amplified by Signal Conditioning Unit 3906, to provide the proper amplitude signal. From Signal Conditioning Unit 3906, the signal may be input into an electrical Demultiplexing Unit (DEMUX) 3908. Based on the signal that comes from processing circuitry 3902 on Select Line 3912, the DEMUX 3908 may transmit the stimulation signal to the corresponding CNTs 3910, which will stimulate the neurons in their vicinity.

Pipeline Summary. An example of a high-level architecture 4000 of the stimulation pipelines described above is shown in FIG. 40. In embodiments, electrical stimulation CNTs may be mixed with optical stimulation and recording CNTs, as there may be little interference between them. As shown in this example, the architecture may include a Serial-In-Parallel-Out converter (SIPO) 4002, a plurality of stimulation pipelines 4004A-M, and zone selection/controller circuitry 4006.

Processing circuitry (not shown) may transmit a serial stream of digital electrical stimulation signals to SIPO 4002. The processing circuitry may translate stimulation commands into a stimulation operation having a particular stimulation signal. SIPO 4002 converts the serial stream to a plurality of parallel digital electrical signals, which may be transmitted to one or more stimulation pipelines 3106A-M. Each stimulation pipeline 3106A-M may convert its input digital electrical signals to electrical or optical neuro stimulation signals 4008, as described above. Neuro stimulation signals 4008 may then be transmitted to zone selection/controller circuitry 4006, which may route each neuro stimulation signal 4008 to an appropriate electrical stimulation electrode or optical stimulation optrode.

Embodiments may contain two units with 100 tiles each. Each tile may contain four selectable stimulation channels which may be controlled independently. In embodiments, up to 400 channels may be used for stimulation at any time. In embodiments, command values may be arranged in a matrix format that corresponds to the physical representation of the stimulation channels. In embodiments, each stimulation command may include the channel reference which represents the address of the optrode that will be used for stimulation. In embodiments, each stimulation command may include the commands array which represents the stimulation values. In embodiments, the commands array may contain the type of stimulation and the stimulation pattern (potential/intensity, timing). In embodiments, the intensity of the light beam may depend upon how far the neuron is in the tissue (and therefore how strong the light source should be in order to reach it). In embodiments, each stimulation command may depend on its specific goal, which will dictate whether the task is to increase or decrease voltage inside the targeted neuron(s). In embodiments, the optical stimulation commands shall specify the features of the stimulation pattern (light wavelength, light intensity, frequency, and duration). In embodiments, the electrical stimulation commands may specify the discrete voltage values to be applied through the stimulation channels at each time step. In embodiments, the command values may be arranged in a matrix format (10×10 commands for tile) that corresponds to the physical representation of the stimulation channels. In embodiments, a DAC may convert the digital signal into an analog signal. In embodiments, a stimulation light may have wavelengths between 400-650 nm. In embodiments, each stimulation command may be encoded as 5 bits, resulting in a total of 32 different possible stimulation commands.

Architecture Overview. An exemplary block diagram of an embodiment of an implant device 4100 is shown in FIG. 41. In this example, implant device 4100 may include neuronal recording circuitry 4102, neuronal modulation or stimulation circuitry 4104, control module/processing circuitry 4106, compression module 4108, closed loop control module 4110, gateway communication module 4112, temperature and power management module 4114, and status and configuration module 4116. In this example, implant device 4100 may further be electrically, optically, and/or communicatively connected to neural tissue neurons 4118 and gateway 4120. It is to be noted that the circuitry shown in FIG. 41 may also include, or be associated with, software to cause the circuitry to perform the desired functions.

Neuronal recording circuitry 4102 may include circuitry, such as that described above, for recording electrical and/or optical signals from neurons 4118. Neuronal modulation or stimulation circuitry 4104 may include circuitry, such as that described above, for generating and transmitting electrical and/or optical stimulation signals to neurons 4118. Control module/processing circuitry 4106 may include circuitry, such as that described above, for receiving data from neuronal recording circuitry 4102 representing recorded electrical and/or optical signals from neurons 4118 and for generating and transmitting command data neuronal modulation or stimulation circuitry 4104 to generate and transmit electrical and/or optical stimulation signals to neurons 4118. Compression module 4108 may include circuitry for receiving recorded data from control module/processing circuitry 4106 and compressing the recorded data. Closed loop control module 4110 may include circuitry for receiving neural recording data and updating stimulation command data based on the received neural recording data to achieve closed-loop control of the stimulation process. Gateway communication module 4112 may include circuitry for communicating data to and from gateway 4120. Temperature and power management module 4114 may include circuitry for monitoring and controlling implant device temperature, power consumption, battery charging and discharging, etc. Status and configuration module 4116 may include circuitry for monitoring implant device status and for managing the configuration of the implant device.

Software Architecture.

Neuronal Recording Interface. Control module/processing circuitry 4106 may make reading requests to the neuronal recording circuitry 4102 specifying the desired sampling rate and the target CNTs. An example of pseudocode for data recording is shown in FIG. 42.

Neuronal Modulation Interface. Control module/processing circuitry 4106 may make neuron modulation requests to the Neuronal modulation or stimulation circuitry 4104. An example of pseudocode for stimulation requests is shown in FIG. 43.

Control module/processing circuitry Input/Output (I/O) Interactions.

Stimulation Scheduler. In embodiments, there are options regarding what circuitry will be responsible for keeping track of the stimulation command duration. In an embodiment, closed loop control module 4110 may be responsible for keeping track of time. In this case, closed loop control module 4110 may send a stimulation command to control module/processing circuitry 4106, which may apply that stimulation recipe until otherwise instructed. An advantage of this approach is that control module/processing circuitry 4106 does not have to feature a function for stimulation time management. However, control module/processing circuitry 4106 still may have to deal with timing issues for recording (the sampling rate).

In an embodiment, the time management function may be implemented in control module/processing circuitry 4106. In this case, closed loop control module 4110 may send a stimulation command to control module/processing circuitry 4106, along with a time period value. Control module/processing circuitry 4106 may apply that stimulation recipe for the specified duration. When the specified stimulation time ends, the stimulation on that channel may stop and the control module/processing circuitry 4106 may waits for further instructions. If a new command is received while the previous one is active, the previous one may be overwritten. The advantage of this approach is that closed loop control module 4110 is entirely free from managing time and can focus on I/O management.

In embodiments, modules may modify the list of active channels for recording, such as closed loop control module 4110 and gateway communication module 4112. Gateway communication module 4112 may modify the list of active channels for recording in order to read a different set of channels than the ones that are in use by closed loop control module 4110.

Throttling Side-channel. Control module/processing circuitry 4106 may also communicate with temperature and power management module (TPMM) 4114. In embodiments, when TPMM 4114 detects that the temperature of the implant device is rising, approaching the thermal safety limits, it may send a SLOW signal to control module/processing circuitry 4106 to start throttling the I/O activity. When receiving the SLOW signal, control module/processing circuitry 4106 may decrease the recording sampling rate and communicate to closed loop control module 4110 to reduce the rate of stimulation commands. If the temperature exceeds the thermal safety threshold, TPMM 4114 may send a STOP signal (by flipping another bit) to control module/processing circuitry 4106, which may then cease all recording and stimulation activities.

TPMM 4114 may also monitor the battery level of the implant device. If the battery level falls below a threshold B1, TPMM 4114 may send a SLOW signal to control module/processing circuitry 4106 to start throttling the I/O activity. If the battery level falls below a lower threshold B2, TPMM 4114 may send a STOP signal to control module/processing circuitry 4106 in order to preserve battery life.

In embodiments, this side channel may be focused only on activity and process control, therefore no neural data may be sent or received on it.

Data Flow. In embodiments, an efficient data flow between the modules may be implemented, which will take into account the constraints in terms of memory and processing resources.

For example, in embodiments, control module/processing circuitry 4106 may place the recorded data in a memory buffer (an array) from which data will be shared with the other modules, according to the protocol described above. Closed loop control module 4110 may store the stimulation commands in a memory buffer (an array) from which the commands may be used by the control module/processing circuitry 4106 for stimulation.

TPMM 4114 may send signals to control module/processing circuitry 4106 by flipping a corresponding bit in memory. This bit may also be shared with closed loop control module 4110 and may trigger the slowing down of the stimulation activities.

Closed-Loop Control (Command & Recording). In embodiments, brain stimulation may be more effective when it is applied in response to specific brain states, via Closed Loop Monitoring, as opposed to continuous, open loop stimulation. An example of a conceptual sketch of a closed loop control system 4400 is shown in FIG. 44. In this example, a target signal 4402, which may indicate a desired output 4410 from system 4400, may be input to system 4400. An error circuit 4404 may determine a difference (error signal) between target signal 4402 and a measurement 4412 of output 4410. The error signal may be input to a controller 4406, which may generate a control input signal 4408 to control system 4409 to generated the desired output 4410 indicated by target signal 4402. Output 4410 may be measured 4412 and feedback to error circuit 4404. In overall operation, closed loop control system 4400 may continuously adjust its operation so that the actual desired output 4410 corresponds to the desired output indicated by target signal 4402.

Closed-loop, activity-guided control of neural circuit dynamics using optical and electrical stimulation, while simultaneously factoring in observed dynamics in a principled way may be a powerful strategy for causal investigation of neural circuitry. In particular, observing and feeding back the effects of circuit interventions on physiologically relevant timescales may be valuable for directly testing whether inferred models of dynamics, connectivity, or causation are as accurate in vivo testing.

In embodiments, Neuronal Response Latency (NRL) may measure a time-lag between the extracellular stimulation and the intracellularly recorded evoked spike. The NRL of the same neuron may vary among extracellular stimulating electrodes depending on their position; however, for a given stimulating electrode it may be reproducible qualitatively (for low stimulation frequencies). For example, the NRL may range between about 1-15 ms.

In embodiments, spike-detecting, closed-loop Single Input Multiple Output (SIMO) control may use template matching to do online spike detection on 32-channel tetrode recordings (system outputs) and may use detected spikes to control optogenetic stimulation through a single fiber optic (system input) at ˜8 ms closed-loop latency in awake rats. Further, simulated closed-loop control in an all-electrical Multiple Input Multiple Output (MIMO) systems for Electrical Deep Brain Stimulation (EDBS) may raise key points directly relevant to closed-loop optogenetics for MIMO systems, showing that a properly designed MIMO feedback controller may control a subset of simulated neurons to follow a prescribed spatiotemporal firing pattern despite the presence of unobserved disturbances. Such disturbances may be typical in neural systems of interest, as most of the brain will remain unobserved. Further, a simplified linear-nonlinear model may be quite effective in controlling firing rates, despite strong simplifying assumptions (this is important for systems where speed dictates hard computational constraints). In addition to the practical goal of safer, more effective deep-brain stimulation, the resulting spatiotemporal patterns identified may themselves be of intrinsic value in providing new insights into how neural circuits process information.

Additional theoretical work may involve optimal control theory to design control inputs that evoke desired spike patterns with minimum-power stimuli in single neurons and ensembles of neurons using electrical current injection. Robust computational models may use similar methods for optimal control of simple models of spiking neural networks and for individually controlling coupled oscillators using multilinear feedback. Given that converging evidence suggests that abnormalities in synchronized oscillatory activity of neurons may have a role in the pathophysiology of some psychiatric disease and considering their established role in epilepsy, it may be fruitful to continue considering oscillations themselves as a direct target of closed-loop optogenetic control alongside control of spiking neurons.

As described above, in closed-loop optogenetics, the control input 4408 may be a structured, time-varying light stimulus that is automatically modulated based on the difference between desired and measured outputs. Measured outputs may include behavioral, electrophysiological, or optical readouts of activity generated by the subject.

In embodiments, optrodes-MEA are may be used as a hybrid approach for optical neuron stimulation and electrophysiological neuron recording. Embodiments may use optical fibers ‘coated’ with CNTs in order to support this hybrid approach, being able to record and stimulate both optically and electrically.

The advantage of optical over electrical interaction with the neurons is that, while electrical stimulation implies current dissipation in the surrounding area, optical stimulation is able to target specific neurons with greater precision, and it incurs minimal interference with simultaneous electrophysiological recording techniques.

Control Techniques. Depending on the specific neural modulation task associated to the disease that is being treated, embodiments may use different closed loop control packages, which may be uploaded to the implant device. These may be implemented in the control module/processing circuitry.

In embodiments, different types of control techniques may be used for closed loop control. For example, such techniques may include simple on/off control, Proportional Integral Derivative (PID) control, Model Predictive Control (MPC), robust control, adaptive control, and optimal control. Each of these techniques may have different tradeoffs, for example, between obtaining more accurate results and being more computationally costly. The control technique may be chosen based on both the available hardware resources and on the task at hand. In embodiments, the closed loop controller module may use a simple on/off technique, or any other closed-loop control technique.

The control technique may rely on machine learning models trained both offline and online. For example, offline, gathered data may be processed in the Cloud with the purpose of deriving new insights for treatment and encapsulated in new models. This task may be advantageously performed remotely from the implant device due to the greater processing power and memory resources that may be available remotely, such as in the Cloud.

Online, the models obtained in the Cloud may be used on the implant for neuron modulation. In this way, computationally costly but necessary processing may be run offline, yielding new models appropriate for fast online conditional stimulation of the neural activity. In addition to the implant device applying the models computed in the Cloud, it may also be able to run simpler machine learning techniques on a dedicated hardware component. However, in embodiments, the models computed offline may have priority over those computed online due to the Cloud's ability to process larger amounts of data and use more advanced machine learning techniques.

In embodiments, models used by the control algorithm may be personalized for each individual user employing transfer learning. A general model may be trained on a large amount of data gathered from a large number of patients and may then be refined by training on data recorded from each individual patient. In this way, each patient may have their own personalized model, with the same generic architecture, but unique weights. Hence, transfer learning may be used to enable use of large amounts of general collected data for the benefit of individual patients and model personalization may be an appropriate approach due to the fact that neural activity has features that are specific to each patient depending on several factors (for example, age, health condition, etc.)

Closed Loop Module. In embodiments, the closed-loop controller module may have a well-defined interface, common to all the controller modules, which may be used to read data and to send commands. In embodiments, the closed-loop controller module may have a simple on/off algorithm, for example, sketched in pseudocode shown in FIG. 45. For example, in the memory improvement task, the calculate_next_state function may run a logistic regression model to predict whether the currently heard word will be remembered, while the calculate_duration function would return a constant duration of X ms.

An example of a PID algorithm is shown in pseudocode FIG. 46. In this example, The KP, KI, KD, and bias are constants that may be tuned for every implant.

Closed Loop Control Conditions. In embodiments, decisions to stimulate taken by the implant device may be sent to the Gateway/Cloud for further processing and fine-tuning of the online model. Due to time constraints (for example, <8 ms latency may be required), the decision to stimulate may be taken internally by the implant device. Using machine learning techniques, the implant device may also compute the optimal optic or electric response that minimizes the difference between current and ideal neural activity. The closed loop control module may monitor voltage levels inside neurons through electrical and optical recording.

In embodiments, the closed loop control module may output the appropriate stimulation pattern in less than 8 ms from when the neuronal measurement was taken. The implant device may allow the Gateway to replace or update the closed feedback loop technique (controller) according to what best fits the task at hand. The task-specific technique may be used to process the recorded data to determine the appropriate stimulation pattern. The closed loop control module may output (to the Stimulation Module) the appropriate stimulation pattern encoded in one of, for example, 32 control commands. All the controller modules may take into account the safety thresholds described below.

Control Module/Processing Circuitry. The raw data as it comes from the CNTs may not be interpreted directly. It may be preprocessed and filtered for noise removal. Before it can be sent to the Cloud, it also may be compressed. Also, for processing with the Closed Loop Control Module, first the state of the neurons(spiking or not) may be identified.

Data Types.

Neuronal Recording. In embodiments, the measured data may be stored in 10-bit variables for both electrical and optical reading. The electrical recording may represent a potential measurement with values between, for example, about −100 mV and 100 mV. These values may be normalized to a floating-point value between [0, 1].

In the case of optical reading, light intensity emitted by the fluorescent substance may be measured. This reading may be correlated linearly with the voltage going through the neuron's membrane and may be represented as between, for example, about −100 mV and 100 mV. These values may also be normalized to a floating-point value between [0, 1].

Neuron Stimulation. In embodiments, stimulation commands may be encoded with 5-bit data. As a result, the implant device may be able to trigger a total of 32 different stimulation patterns. For example, the first bit may specify the type of stimulation (electrical or optical), and the last 4 bits may describe the actual patterns, resulting in 16 combinations for each type of stimulation. In the case of electrical stimulation, the patterns may vary in terms of applied electrical potential and timing. In the case of optical stimulation, the patterns may vary in terms of light wavelength, intensity, and timing.

Data Buffering. The compression module may process blocks of recorded data, hence, in embodiments, the recorded values may be buffered until an entire block is filled. The required size of the input buffer may be at least 100*10=1000 bits=125 bytes.

In embodiments, for the output, a second buffer may account for any potential problems in data transfer to the Gateway, such as packet loss over the Wi-Fi signal or unexpected transfer rate changes. Using a buffer for the output channel may also make the transfer process more robust, as sending data may be more efficient if data is first gathered in a data frame before being transferred to the recipient. In embodiments, the minimum required buffer size may be determined by the size of the largest Wi-Fi frame, for example, 2304 bytes.

Spike Sorting. An exemplary data flow block diagram of a spike sorting technique 4700 is shown in FIG. 47. As shown in this example, when data arrives in a data buffer 4702, spike detection 4704 may be performed, using, for example, an adaptive threshold 4706 to recognize spiking events, template memory 4708 to identify neurons, and correlation detector 4710 to identify overlapping spikes.

The obtained spiking data may then be compressed 4712 so that it can be buffered 4714 and sent. In the spiking compression process predictive filters 4716 may be used to correct for potential erroneous measurements and Run Length Encoding 4718 and Huffman Coding 4720 may be used to compress the data encoded in zeroes (for when neurons are not spiking) and ones (when neurons are spiking).

In embodiments, the electrical potential data recorded from the CNTs may contain signals from multiple nearby neurons. Many neurons, however, have a distinctive spiking pattern, which enables their identification from these recordings. The neurons that are the closest (up to, for example, about 100 microns) to the CNT tip may be identified individually, while for neurons that are between, for example, about 100 and 150 microns, their spikes may be detected, but the background noise may be too strong for individual identification.

Noise Filtering. In embodiments, the first step in processing the data may be to apply a filter in order to remove noise. A band pass filter between 300 and 3000 Hz may be employed for electrical signals recorded from neurons.

Spike Detection. In embodiments, a spike may be detected when the electric field potential exceeds a given threshold. Because different neurons have different thresholds, the threshold value may be set through an adaptive method. For example,

$$Thr = 5 {σ}_{n}$$

$${σ}_{n} = median \{ \frac{\vert x \vert}{0.6745} \}$$

Where x is the bandpass filtered signal and σₙ is an estimate of the standard deviation of the background noise.

Feature Extraction. In embodiments, using wavelets to extract features from the raw waveforms may result in a better separation of the clusters for the templates. The wavelet coefficients may be selected so that they have a multimodal distribution, to be able to distinguish different spike shapes. This may be performed using, for example, a Kolmogorov-Smirnov test for Normality.

Clustering. In embodiments, in order associate the spikes to the neurons that produced them, clustering may be performed on the resulting data. For example, the Super-Paramagnetic Clustering (SPC) method may be used. SPC is a stochastic method that does not assume any particular distribution of the data and groups the spikes into clusters as a function of a single parameter, the temperature. In analogy with statistical mechanics, for low temperatures all the data may be grouped into a single cluster and for high temperatures the data may be split into many clusters with few members each. There is, however, a middle range of temperatures corresponding to the super-paramagnetic regime where the data may be split into relatively large size clusters, each one corresponding to an individual neuron that is recorded.

An example of pseudocode for performing an SPC method is shown in FIGS. 48a-b.

In embodiments, the clustering process describe above may be performed offline, for example, in the Cloud, and only the resulting neuron templates may be communicated to the implant, which may use them to detect new spikes in real time.

Potential challenges are represented by overlapping spikes, which happen when two close-by neurons fire at the same time. In this case, the two spikes might not be cleanly separable and a different method may be to solve this problem, such as looking for linear superpositions of other spike shapes. An example of pseudocode for such a Spike Sorting technique is shown in FIG. 49.

Data Compression. In embodiments, the implant device may generate up to 400 Mb/s of uncompressed data, which may exceed the bandwidth capabilities of low powered wireless transmission methods. Accordingly, in embodiments, the data may be compressed. Two major types of compression techniques may include lossy compression and lossless compression. The advantage of the lossless compression is that the raw data may be exactly reconstructed in the Cloud, but the compression ratio (around 2-3× at most) may not be as large as the one available with the lossy methods. With lossy compression, the original data cannot be reconstructed exactly. There is a tradeoff to be made between how much data is lost and how strong the compression is. Embodiments may use lossy compression, lossless compression, and/or a combination of the two techniques.

Optical and Electrochemical Data. In embodiments, Discrete Wavelet Transforms (DWT) with run-length encoding may be used to compress data in a lossy manner. In embodiments, using compression sensing with unsupervised dictionary learning may result in compression rates between 8× to 16×, with a signal to noise distortion ratio (SNDR) between 3.60 dB and 9.78 dB.

Electrical Recording Data. In embodiments, the methods described above may be used when the goal is to preserve the waveforms of the spikes. For a higher compression ratio, but at the cost of losing raw waveform information, embodiments may use spike detection and/or spike sorting. Examples of hardware implementations of spike detection may be as simple as a comparator with a pre-defined threshold. In this way, a compression ratio higher than 100× may be achieved with little power consumption.

In embodiments, bit encoding techniques may be applied to detected spikes. The activity wave may be segmented in X regions and then bit encoding may be used for each region. For example, if the activity range is split into 16 regions, the values may be encoded in just 4 bits instead of 10 bits. Then, the recording of each channel may be encoded in a fixed position in a block array. Each recording channel value (4 bits) may be a part of a time block container.

An example of bit encoding techniques for the case of 1 bit per channel is shown in FIG. 50. In embodiments, this technique may be extrapolated to, for example, 4 bits per reading channel. An example of a Python implementation of bit encoding techniques is shown in FIGS. 51a and 51b.

Example. For a better understanding, the following example is presented. In this example, there may be 1000 reading channels. In each channel, the recorded values may be encoded as 4 bits, with a sample rate of 10,000 samples per second. For sending data blocks, each taking 10 ms to transmit, a matrix may be generated wherein the bytes for each row is 1000 channels×4 bits/channel=4000 bits/8=500 bytes. The number of rows is 10 ms×20.000 sps/1000 channels=200 Rows. Thus, in total, the Header Information—containing the timestamp for time TO may include a Header Marker of 2 bytes, the Timestamp for TO of 4 bytes, and a Data Size of 200 Rows×500 bytes=100,000 bytes.

In this specific example, 100,006 bytes, which include 1000 channels recorded at an interval of 10 ms, may be transmitted. If a compression of 100× is achieved, a data buffer of only 1 KB may be needed for each 10 ms span representing the recorded data from 1000 channels.

Running Modes. In embodiments, the Spike Sorting and Data Compression Modules may learn from recorded data in the first stage. Therefore, in embodiments, after being implanted, the implant device may run in a training mode for a period of time, at the end of which it may switch to an operating mode. If, in operation, the implant device configuration produces greater than some predefined level of errors, when evaluated by an evaluation model, than the implant device may switch back to training mode for re-configuration. The evaluation model may be configured depending on the specific task of the implant device. Such could be the case, for example when the implant device drifts, making the recorded data no longer match with the previously learned neuron spiking patterns.

For example, an evaluation metric that may be used to switch back to training mode may include determining if the number of spikes per minute, averaged over N hours or during a known supervised exercise, drops below X % of the initially recorded number of spikes per minute. If so, then the implant device may switch back to training mode to learn new dictionaries and new spike templates.

In embodiments, during the training phase, the implant device may collect the raw waveforms and send them to the Cloud for acquisition of the dictionaries for the data compression (if lossy compression is used) and for generation of the spike templates that may be used for the spike sorting process. Most of the lossy compression methods work by building a list of the most often repeated parts of the data, which may be stored in a dictionary. Then, the whole data may be scanned for parts that are very close to the entries of the dictionary and they may be replaced by a pointer to them. In this way, several bytes may be replaced with just a pointer into the dictionary. In embodiments, Cloud processing may be used create the dictionary that may be used for compression by the implant device. The lossy factor may be represented by how the similarity between the scanned data and the dictionary entries is modeled.

When enough data has been collected—meaning that the models perform to a specified task dependent accuracy threshold—the implant device may switch to the operating mode. In this mode, the implant device may perform the following actions: identifying active neurons and recording spiking activity, running the closed loop feedback controller module trained and computed on the Cloud, and compressing the recorded data and sending it to the Cloud.

In embodiments, besides the training and operating modes, the implant device may also be configured in terms of the data transfer ratio and compression method. In embodiments, examples of configurations that may be used include:

All the channels recording electrical signals. Data compression may be based on spike detection and bit encoding, transmitting only neural spike timings to the Cloud without any raw waveforms.

Only a number of N channels may be used in total for recording, in any of the three recording modes. Lossy compression may be performed on the waveforms and the resulting data may be sent to the Cloud. In this case, the maximum number of channels depends on the quantity of data that must be preserved during compression.

Less than 5% of the channels may be used in total for recording. The compression may be lossless and the full waveform may be reconstructable in the Cloud.

In embodiments, when the transfer rate is lower than the recording rate, the implant device may use appropriate techniques to filter the data to obtain a manageable data volume. The implant device may perform real time Nx compression of the recorded data. The N value may be defined depending on the hardware limitations and task goals. In embodiments, the implant device may have an input buffer for the neuron recordings of at least 125 bytes. In embodiments, the implant device may have an output buffer of at least 2304 bytes. In embodiments, the implant device may have two running modes: training mode and operating mode. In embodiments, the implant device may be able to classify spikes to identify which neuron they belong to. In embodiments, the implant device may test different lossy and lossless compression algorithms, with the goal of choosing the optimal method. In embodiments, the implant device may initially start in training mode. In embodiments, the implant device may be able to switch between running modes upon receiving a command from the Gateway.

Gateway Interface. In embodiments, the implant device may be wirelessly connected to a Gateway component by exposing an interface for the following processes: Transmitting neural recording streams, receiving control commands, and Receiving configuration commands. Embodiments may use wireless communications such as Wireless Data Communication Type—802.11ac, Wireless Frequency—5 GHz, and Radio Channel Size—80 MHz.

In embodiments, Wi-Fi communications may be used, due to its high rate data transmission. However, embodiments may use alternatives to the 802.11ac Wi-Fi standard. For example, the Full-Duplex Wireless Integrated Transceiver for Implant-to-Air technology may be used. This technology includes a transmitter designed to support uplink neural recording applications with a data rate of up to 500 Mb/s and power consumption of 5.4 mW and 10.8 mW, respectively (10.8 pJ/b). This high-speed data transfer rate removes the need for compression in the implant device, which may reduce the overall power consumption and generated heat. Also, another advantage of this chipset is its size of just 0.8 mm×0.8 mm. Another example is the Thread Protocol, an IEEE 802.15.4 standard, which provides a data transfer rate of 250 Kbps. This technology may have advantages including great community support, low power consumption, supported by a large number of chipsets manufacturers, secured, and stable implementation.

In embodiments, the communication channel between the implant device and Gateway may include Bluetooth. This may be the case, for example, when the Gateway device is a smartphone. In order to accommodate this requirement, the implant device may be able to buffer the data transmitted to the Gateway in cases when the transfer speed is lower than the recording speed.

In embodiments, the recorded data may be encoded as a 10-bit floating point value. Given that most of the AI and Processing Tools on the Cloud Component are processing float data in 16 bits or 32 bits encoding, the input data may be converted in the Cloud to the corresponding data type.

In embodiments, a default version of software may be installed at the factory. In embodiments, the implant device may start with a provisioning procedure if the provisioning was not already done. In embodiments, the implant device may support over-the-air (OTA) updates. In embodiments, the software may be constantly updated with novel processing models to ensure its integrity and proper functionality for the specific performed task. These updates may be performed after implantation in the brain. In embodiments, the OTA update interface may be dependent on the hardware specifications. Embodiments may allow updates to be pushed over the wireless communication channel only from specific IP address. In embodiments, stimulation and recording operations may be paused during the OTA updates. In embodiments, the implant device may restart the recording and stimulation operations, after the OTA update is finished. In embodiments, the updates may preserve the integrity of implant device. In embodiments, if an OTA update fails for any technical reasons, the implant device may restart and continue to use its previous Software Version. In embodiments, OTA updates may be processed only when the battery level is higher than a threshold that guarantees a safe update and restart of the implant device. In embodiments, OTA updates may be accepted only from one or more specific configured Gateways. In embodiments, automatic OTA updates may be enabled/disabled through configuration parameters.

In embodiments, when the implant device is initially powered on, it may start a private WAN by initiating an AP (Access Point). The Gateway may connect to this AP, using a password that is specific to the target implant device, such as a serial number or other unique identifier. In embodiments, after a successful connection, the Gateway may initiate the Provisioning Phase. In embodiments, the Provisioning Phase may provide the default parameters for all the initial configurations of the target implant device. In embodiments, the initial configuration may include parameters such as predetermined MAC addresses of the accepted Gateways, power system configuration parameters, local WAN credentials, recording parameters, wireless charging parameters, configured blocks for reading, etc.

In embodiments, after the provisioning phase is finished, the implant device may execute a reset command. After the implant device has restarted, it may connect to the local WAN, being ready to receive new commands from the Gateway. In embodiments, if Wi-Fi/AP Provisioning is not supported, for example, with mobile devices, the implant device may use the Bluetooth channel for provisioning. In embodiments, when the implant device is initially powered on, it may start the Bluetooth Discovery process, to perform Bluetooth Low Energy (BLE) pairing with the Gateway. In embodiments, the implant device may use a fixed value pin for pairing that shall be linked to the target implant device. In embodiments, after the pairing operation is executed successfully, the same Wi-Fi provisioning steps mentioned above may be performed.

In embodiments, the Gateway may connect to the implant device using a secured configuration interface. In embodiments, the Gateway may have the rights to modify configuration parameters such as power system configuration parameters, wireless charging parameters, recording parameters, activated recording blocks, activated recording channels per block, etc. In embodiments, for security reasons, the MAC addresses of the valid gateways may not be changed via the Configuration Interface. Rather, they may be changed only via the Provisioning Configuration Process.

In embodiments, a Gateway may connect to one or multiple implant devices. In embodiments, a Gateway may save the data stream from all connected implant devices. In embodiments, the implant device may only accept as a Subscriber to its Published data the Gateway which has the MAC address that was configured during the Provisioning Phase. In embodiments, the communication channel between the implant device and Gateway may support continuous data streaming of, for example, up to 4 Mb/s.

In embodiments, the implant device may publish its recorded data when it is requested by the Gateway. In embodiments, the Gateway may receive the real time data from the implant device through a secured data streaming protocol. In embodiments, in the data streaming process, the Gateway may act as the receiver, while the implant device may be the publisher. In embodiments, the implant device may switch off the data transmission as long as there is no Gateway connected, for battery conservation. In embodiments, every sample time the implant device may apply a framing mechanism to create a data frame consisting of Header Marker and a Payload. In embodiments, the Header Marker may be used to mark the boundaries of the current frame. In embodiments, the Payload may be calculated as follows: A×N×CBS, where A is the number of activated reading blocks (up to, for example, 100), N is the number of activated recording channels per block (up to, for example, 10), and CBS is the size of the compressed reading per channel.

In embodiments, the implant device may have a Data Transfer Buffer placed between the PISO layer and the Communication Channel. In embodiments, the Data Transfer Buffer may be used in cases when the transfer rate falls below 4 Mb/second. In embodiments, the implant device may receive control commands from the Gateway. In embodiments, each individual stimulation command may be encoded in 2 bytes, containing, for example, a reference to the blocks that are closest to the targeted neurons (for example, 8 bits), the referenced channel inside the block (for example, 2 bits), and the desired encoded stimulation command from the, for example, 32 different stimulation patterns (for example, 5 bits).

In embodiments, individual stimulation commands may be grouped together to be executed simultaneously. In embodiments, for each tile block, for example, 1 up to 4 channels may be stimulated simultaneously. In embodiments, a stimulation command may have a size up to for example, 200×2 bytes.

In embodiments, the communication between the implant device and the Gateway may be secured. The implant device and Gateway Wi-Fi chipset may provide a hardware secure channel between these two devices. In embodiments, in order to react promptly to the recorded data, the implant device may use machine learning models for data processing. The models may be trained in the Cloud and then pushed to the implant device via the Gateway. In embodiments, the implant device Gateway Communication Module may request the machine learning models from the Gateway through a dedicated application programming interface (API). In embodiments, the control module/processing circuitry and the closed-loop control module may load the models and may use them for data processing. In embodiments, the machine learning models may be updated using the OTA updates. In embodiments, the implant device may receive activation and inactivation commands over the stimulation API. In embodiments, the implant device may receive status request commands, and may respond with information such as battery level, temperature level, software version, enabled reading/stimulation tiles, device state, etc.

A pseudocode example of a Startup Procedure is shown in FIG. 52. A pseudocode example of a Provisioning Procedure is shown in FIG. 53. A pseudocode example of a Configuration Interface is shown in FIG. 54. A pseudocode example of a Stimulation Interface is shown in FIG. 55. A pseudocode example of a Recording Interface is shown in FIG. 56. A pseudocode example of a Status Interface is shown in FIG. 57.

In embodiments, circuitry of the implant device may meet certain specifications, for example, in terms of CPU, RAM, and I/O characteristics.

CPU Speed. In embodiments, CPU speed may meet certain specifications. For example, the implant device may be able to record up to 20,000 samples per second. Each sample may be encoded in 10 bits. There may be 1000 channels per integrated circuit in the implant device. Accordingly, the transfer size per second may be calculated as 20,000 samples/second*10 bits/sample=200 Kbits/second/channel. 200 Kbits/second*1000 channels=2*10\hat{ }8 Bits/second=200 Mbits/second. Assuming that 100 operations (machine instructions) are needed for compressing one 32-bit integer, the number of operations needed for compressing one packet of 200 Mbs may be calculated as (2*10\hat{ }8 bits to process/32 bits per int)=6,250,000 Bits/operation; 6,250,000 Bits/operation*100 operations/int=625,000,000 operations. Assuming 1 operation per cycle, a CPU clock speed of at least 625 MHz may be needed.

For example, 1000 million integers may be compressed per second with Single instruction, multiple data (SIMD) acceleration. This results in approximately a 3× compression. Assuming a duration of one cycle per instruction, on a 3 GHz processor, compressing one integer would take 3*10\hat{ }9/10\hat{ }9=3 instructions. Given that SIMD instructions typically work on a 128-bit (16 bytes) architecture, with an 8-bit architecture, approximately 3*16=48 instructions may be needed to compress an integer. Taking into account the adjacent processes of copying data into memory and running the Closed Loop Module simultaneously, the 100 operations per integer are justified.

RAM Memory. In embodiments, RAM requirements may be estimated. Assuming a compression ratio of 10 and an Output Buffer of 2304 bytes (a limitation of the maximum packet frame supported by Wi-Fi. Therefore, the size of the Input Buffer that will store the data that needs to be compressed to the Output Buffer size will be ten times larger: 10*2304=23040 bytes. Further, the input and output buffers may be doubled to avoid synchronization issues between the reading and writing processes. In addition, in embodiments, a third intermediary buffer may be added of the same size as the Output Buffer, which may be used for storing other relevant data needed for the computation. Accordingly, an example of a formula for minimum total RAM size requirement is 3*(23040+2304)=76032 bytes=76 kB.

In embodiments, there may be a need for other RAM uses for example, running machine learning models, commands and status communication with the Gateway, etc.

I/O Interface. In embodiments, the implant device may support 802.11ac Wi-Fi and Bluetooth Low Energy connectivity for transmitting data to the Gateway. In embodiments, to connect to the CNT layer, the implant device may also have at least 26 general purpose I/O pins. For example, 12 pins may be used for controlling the MUX Select Lines when recording data, 12 pins may be used for controlling the DEMUX Select Lines in stimulation commands, and two more pins may be used for the actual data transfer.

Device Size. In embodiments, the chipset size used for the implant device may be, for example, about 15 mm×15 mm. A number of currently available processor chips or chipsets meet this size, and some of them also provide the necessary CPU and RAM characteristics. Some also support Wi-Fi/BLE, but there are small chips that could be used for this functionality.

Temperature & Power Management. In embodiments, the implant device may constantly monitor its temperature and power levels in order to make sure it doesn't damage brain tissue. When the implant device detects that temperature levels are starting to rise, it may throttle the neural recordings and stimulations. If the temperature increases by, for example, about 1° C., the implant device may stop all recording and stimulation activities and all processing until the temperature is back to normal.

In embodiments, when the implant device detects that the battery levels are getting low, it may enter a battery saving mode, where neural recordings and stimulations may be throttled. If the battery level reaches a critical threshold, for example, under about 10%, all recordings and stimulations may be stopped, to prevent the implant device from discharging completely.

In embodiments, the implant device may also keep track of the total power output into the brain. Thermal limit requirements inside the brain may be <1 mW/mm². This limit may not be exceeded. As a safety threshold, throttling may start when power output is over 0.75 mw/mm². In embodiments, due to health and safety reasons, electrical stimulation potentials may be below the threshold of 700 mV at all times. An example of pseudocode for the temperature and power monitoring module is shown in FIG. 58.

Safety Thresholds. In embodiments, the implant device may limit its worst-case temperature rise (due to a local hot-spot) from 1° C. to 0.8° C. The typically accepted limit up to which a compact device may be allowed to heat up without damaging surrounding brain tissue is 1° C., so embodiments may provide additional safety margin.

The electrical stimulation potentials threshold for irreversible tissue damage is generally considered to be at 700 mV. Therefore, in embodiments, the implant device may limit electrical stimulation potentials to 700 mV. In order to stay below this threshold while still reaching the desired volume of tissue, embodiments may use multiple current release sites.

The Gateway. In embodiments, the implant device may be connected to the neurons, being able to read data and execute stimulation commands on them. Data received from the implant device may be analyzed by researchers and doctors. By using AI/ML models, the doctors may command different stimulation patterns for neurons from different brain areas in order to treat different brain related diseases.

In embodiments, the implant device may stream data up to 4 Mb/s. Pushing all these data directly to Cloud would require either a high band internet connection or a large buffer on the implant device. Both options may have disadvantages such as high costs, limited hardware resources, battery consumption etc. Also, the data content may be highly sensitive, which may require the data to be sent over a highly secured channel that may provide the consistent delivery and privacy of the data.

Responsibilities. Accordingly, in embodiments, the implant device may communicate directly with a Gateway component. The responsibilities of the Gateway may include receiving high speed data stream from the implant device, buffering the implant device recorded data, compressing the data and streaming the data securely to the Cloud for processing and analysis, receiving complex control commands from the Cloud and delivering the commands to the implant device as neuron stimulation commands, sending configuration commands to the implant device, and requesting the implant device status information.

In embodiments, the implant device may be provisioned to stream data and to receive commands from only one single Gateway. In embodiments, the Gateway may have the capacity to receive data and send commands to multiple implant devices.

In embodiments, the Gateway may have sufficient processing power to handle the communication with multiple implant devices and to stream data to the Cloud and to receive commands from the Cloud. To reduce the complexity of the Gateway and to reduce the maintenance efforts, in embodiments, the Gateway may not contain complex logic or a complex User Interface. The only needed User Interface may be a Configuration/Maintenance Interface.

Examples of Types of Gateway. In embodiments, the software may run on gateway devices such as a mobile gateway, such as a smartphone, tablet, or wearable device, a home gateway, and a deep clinic (hospital) gateway. In embodiments, each of the gateway types may use the same data transfer and security protocols, but may allow for different data rates, buffering and analysis tools, and may have different associated implant device operation modes.

In embodiments, Gateway hardware may include, for example, a CPU/Main Board—for example, ready for operating system kernel installation, a Wireless Communication chipset, Wireless Card for connecting to a local Wi-Fi network, Internal Memory>2 GB, Internal Mass Storage>10 GB, etc.

In embodiments, a Gateway software configuration may include, for example, an operating system, Gateway Software, Web-Server software, that may, for example, be use for configuration purposes, etc.

In embodiments, a default version of the Gateway software may be installed on the Gateway from the factory. In embodiments, the Gateway may start with the provisioning procedure if the provisioning was not already done.

In embodiments, the Gateway software may be frequently updated with novel software versions to ensure data integrity and optimal functionality. In embodiments, the OTA updates may be triggered via Cloud commands. In embodiments, during the OTA updates, the Gateway may suspend the connection to implant devices and to the Cloud to be able to properly execute the OTA update. When the update is finished, the Gateway may restart and reconnect to implant devices and the Cloud. In embodiments, OTA updates may not alter the previously configured parameters. In embodiments, OTA updates may preserve the integrity of the Gateway. In embodiments, when the OTA update fails for any technical reasons, the Gateway Module may re-start and use the previous software version. In embodiments, OTA updates may be accepted only from a specific Cloud host and may be signed with a special OTA related key. In embodiments, automatic OTA updates may be enabled/disabled through the use of the configuration API.

In embodiments, during the initial power up, the Gateway may start its private WAN by initiating an AP(Access Point). In embodiments, in provisioning mode, Gateway may start a web-server that may be used to receive provisioning commands. In embodiments, while in the provisioning phase, a user connected to the AP initiated by Gateway may access the Gateway Configuration Interface via a browser. Example of provisioning parameters may include a connection address of the Cloud Host, Cloud connection credentials for the initial configuration cycle, Credentials needed to connect to a local Wi-Fi network, Gateway administration credentials, etc.

In embodiments, once the Cloud Host address and initial credentials are set correctly, the gateway may trigger a “pairing command”. As a result of the pairing command, the cloud may generate an 8-byte code. This code may be set using the Gateway Provisioning UI. The code may be transmitted to the Cloud to prove its identity. After a successful execution of this process, the Gateway may be ready to receive commands from the Cloud and to stream data to the Cloud.

In embodiments, a Local Configuration Interface may be available during the entire period that the Gateway is running for maintenance purposes. In case of malfunction, a technician may connect to this interface, analyze the status and configuration of the Gateway, and determine the cause of the problems. In embodiments, the technician may manually change the configuration parameters. Any manual changes of the configuration parameters may be synchronized with the Cloud.

In embodiments, the Gateway Configuration UI may be implemented as secured web application. In embodiments, the administration credentials may be set only during the provisioning phase or by a credential override command received from the Cloud. In embodiments, the Gateway may expose a configuration workspace without a user interface and the technician could connect for configuration using a mobile application.

In embodiments, after a successful provisioning, the Gateway may register itself as command executor, for the commands sent by the Cloud. Thus, the Gateway may receive any commands sent by a Cloud user for the purpose of commanding or configuring the implant device or the Gateway. In embodiments, once registered as a command executor, the Gateway may receive commands such as a Gateway configuration command, an implant device configuration command, an implant device state inactivation/activation command, an implant device stimulation command, an implant device status command, an implant device OTA command, an implant device control recording command, etc.

In embodiments, for each Gateway configuration command received from the Cloud, the Gateway may validate it and then change the configuration as requested. In embodiments, the data recording from the implant device modules may not be affected, by the execution of configuration commands on Gateway. In embodiments, for each implant device configuration command received from the Cloud, the Gateway may connect to the targeted implant device configuration API, and send the configuration command to that implant device. In embodiments, the implant device configuration commands received from Cloud may be translated to implant device configuration commands before being delivered to implant device over the implant device configuration API.

In embodiments, for each implant device activation/inactivation command received from the Cloud, the Gateway may connect to the targeted implant device stimulation API and then send the activation/inactivation command. In embodiments, the implant device activation commands received from Cloud may be translated into implant device activation commands before being delivered to implant device over the implant device stimulation API. In embodiments, for each implant device stimulation command received from the Cloud, the Gateway may connect to the targeted implant device stimulation API and then send the stimulation command. In embodiments, the implant device stimulation commands received from Cloud may be translated into implant device stimulation commands before being delivered to implant device over the implant device stimulation API. In embodiments, for each implant device status command received from the Cloud, the Gateway may connect to the targeted implant device status API, request the status, and send it back to the Cloud. In embodiments, the implant device status information may include information such as Battery Level, Recording State: on/off, Active Recording channels, Active Stimulation channels, Software version, etc.

In embodiments, for each implant device OTA command received from the Cloud, the Gateway may connect to the targeted implant device OTA API and deliver the software updates. In embodiments, for each implant device Control Recording command received from the Cloud, the Gateway may send to the target implant device the command for execution, for example, start or stop recording. In embodiments, the communication channel between implant device and Gateway may support continuous data streaming of up to 4 Mb/s. In embodiments in which each Gateway may be connected to multiple implant devices, parallel processing of the incoming data streams may be performed. In embodiments, the Gateway may be able to record multiple incoming data channels and to stream them separately to the Cloud.

In embodiments, the communication between the implant device and the Gateway may be secured. The implant device and Gateway Wi-Fi chipsets may ensure a hardware secure channel between these two devices.

In embodiments, the data recorded by implant device may be streamed at a speed up to 4 Mb/s. For such a high rate data transfer to the Cloud, embodiments may include a high-speed data connection. This may become a constraint in different clinics or facilities. Thus, in this scenario, the Gateway may need to handle a high-speed data publisher (the implant device) and a slower consumer—the upload stream to the Cloud. To solve this problem, in embodiments, the Gateway may buffer the data received from the implant device, package and compress it and only afterwards send it to the Cloud at the optimal provided transfer rate.

In embodiments, the Gateway may send to the Cloud data packets of similar sizes. In embodiments, the Gateway may start to send the data when the internal in-memory data buffer is full.

In embodiments, the data coming from the implant device may be compressed using an encoding algorithm. Still, the need to convert, for example, 10 bits float to 16 bits float, enlarges the data volume that needs to be transferred to the Cloud by 60%. To keep the transfer size low and to reduce the Cloud upload latency, the Gateway may compress these data before uploading it to Cloud.

Given that there could be multiple Implant devices connected to the same Gateway, in embodiments, the Gateway may be able to handle the incoming data in multiple parallel threads. The ongoing data transmission flow may not be affected by new incoming data streams. In embodiments, any incoming data channel for a specific implant device may be processed, compressed, and streamed to the Cloud independently of any other active data channels corresponding to other Implant devices.

In embodiments, when the Gateway is powered on, it may open the data incoming channels (server sockets) for all linked implant devices. It may be that for certain reason, for example, battery drain, implant device location changed, etc., the implant device may not be able to connect at that moment to the Gateway. Still, when the implant device enters the connection area and starts transmitting data, the Gateway may pair with the implant device and start receiving its data.

In embodiments, after the provisioning phase is finished, the Gateway may be paired with the Cloud, thus for each implant device that it controls it may, for example, register itself as a Commands Executor and initialize the Data Publisher Channel. In embodiments, any communication between Gateway and Cloud may be over a secure channel and may use an AES(128 bits) encryption key. In embodiments, execution/configuration commands received from Cloud may be encrypted with this key. In embodiments, the Gateway may encrypt all data pushed to the Cloud with the AES key. In embodiments, the AES keys may be periodically changed and may be transferred between Cloud and Gateway using, for example, the Diffie-Hellman Symmetric Key Exchange protocol.

In embodiments, the Gateway may ensure that any data recorded from the implant device may be transmitted to the Cloud. In embodiments, in case of communication failures between the Gateway and the Cloud, the Gateway may retry sending the data when the connection is restored. In embodiments, the Gateway may store locally (on persistent storage) the un-sent data in case the communication channel is broken for a longer period of time. In embodiments, the persistence buffer may have a pre-configured size. In embodiments, once this size is exceeded, the Gateway may apply a first-in-first-out (FIFO) eviction policy. Thus, the older entries may be deleted in order to make room for new incoming data. In embodiments, this may be the only configurable scenario in which the Gateway may lose data received from the implant device. In embodiments, once the connection is re-established the Gateway should automatically synchronize the data with the Cloud.

In embodiments, the data uploaded from Gateway to Cloud may not contain any private information about the patient. In embodiments, the link between the patient details and the recorded data may be stored and known only in the Cloud. In embodiments, each data incoming channel on the Cloud may be associated with a specific implant device. In embodiments, in the Cloud there may be a privacy information database, which may store the relations between the patient and the implant devices. In embodiments, no patient sensitive data may be transferred from Cloud to Gateway. In embodiments, the commands sent from the Cloud may address directly the implant device and may not contain any patient information.

A pseudocode example of a startup procedure is shown in FIG. 59. A pseudocode example of a Provisioning procedure is shown in FIG. 60. A pseudocode example of a command execution procedure is shown in FIGS. 61a, 61b, and 61c. A pseudocode example of a data streaming procedure is shown in FIG. 62.

An exemplary block diagram of a Gateway 6300 is shown in FIG. 63. As shown in this example, Gateway 6300 may include communications with implant device 6302, communications with the Cloud 6304, a data recording interface 6306, data compression 6308, a buffer 6310, a data publisher 6312, a stimulation interface 6314, a command executor 6316, and a configuration/status interface 6318. Communications with implant device 6302 may include hardware and software to provide communications with the implant device. Communications with the Cloud 6304 may include hardware and software to provide communications with the Cloud. Data recording interface 6306 may include hardware and software to receive data from the implant device and process the data prior to data compression, as described above. Data compression 6308 may include hardware and software to provide compression of the processed data received from the implant device, as described above. Buffer 6310 may include hardware and software to provide temporary storage of compressed and/or uncompressed data, as described above. Data publisher 6312, may include hardware and software to publish and communicate data to the Cloud, as described above. Stimulation interface 6314, may include hardware and software to generate stimulation commands, and/or multiple or sequences of stimulation commands to be transmitted to the implant device, as described above. Command executor 6316, may include hardware and software to receive stimulation commands 6320 from the Cloud and execute those comments in conjunction with stimulation interface 6314 and the implant device, as described above. Configuration/status interface 6318, may include hardware and software to receive and process configuration/status commands from the Cloud, as described above.

The Cloud. Data recorded from the implant device may be processed and analyzed. Based on this data, the neuroscience researchers may build AI/ML, models that may be used by practitioner doctors to treat different brain related maladies such as Parkinson, Alzheimer, etc.

The Cloud may include of a cluster of nodes on which different microservices may be deployed. An exemplary high-level block diagram of the Cloud 6400 is shown in FIG. 64. Also shown in this example are implant device 6402 and Gateway 6404. As shown in this example, Cloud 6400 may include a command service 6406 and a data service 6408. Command Service 6406 may receive, for example, stimulation, activation, configuration, provisioning commands from the user via a User Interface and then may distribute them to the Gateways for execution. Command Service 6406 may also receive back the result of the command execution and present them to a user. Data Processing Service 6408 may take care of data ingestion coming from the implant device and the processing and storing of this data.

Command Service. In embodiments, Command Service 6406 may execute commands such as implant device OTA, implant device Configuration, Gateway Configuration, implant device stimulation, implant device activation/inactivation, implant device recording control, etc.

In embodiments, the commands may be transmitted from the Cloud as a request of a user (Medical Doctor, Researcher) and may reach an implant device which may be located in a local network behind a firewall. Accordingly, in embodiments, a Publish/Subscribe architecture may be used. In embodiments, the Cloud may publish commands for execution, while the Gateway may be registered as a subscriber for these commands. In embodiments, the Gateway may, in this case, play the role of commands executor.

In embodiments, Command Service 6406 may be implemented as a microservice and may be deployed on multiple nodes in Cloud 6400. In embodiments, Command Service 6406 may expose an interface for command requests, which may be used by other services to send commands. In embodiments, each command may indicate the implant device or the Gateway to which it is addressed. In embodiments, when a user triggers a command from the user interface, the command may be created and then may be published on a commands Queue. The Command Executor which is registered for that implant device or Gateway Address may execute the command. A pseudocode example of a command message is shown in FIG. 65.

In embodiments, a Configuration Command may contain configuration changes which apply to the targeted implant device. In embodiments, the Configuration Command may include Configuration Parameters that may contain parameters that may be configured on an implant device. In embodiments, the Configuration Parameters may contain information such as Gateway IP/MAC addresses, Stimulation channels, recording channels, Recording reporting frequency, Scheduled start/stop, Stimulation methods—Optical, Electrical, Chemical, etc. A pseudocode example of a Configuration Command is shown in FIG. 66.

In embodiments, the Stimulation Command may include information about the stimulation of specific channels of the targeted implant device. A pseudocode example of a Stimulation Command is shown in FIG. 67. In embodiments, the Command Executor may apply the required stimulation command on the specified channels.

In embodiments, the Activation Command may include information about the activation/inactivation of certain channels of a targeted implant device. A pseudocode example of an Activation Command is shown in FIG. 68. In embodiments, the Command Executor may apply the required activation/inactivation on the specified channels.

In embodiments, the OTA Command may include information about a new version of software that needs to be installed on the implant device. A pseudocode example of an OTA Command is shown in FIG. 69. In embodiments, when executing this command, the gateway to which the implant device is connected may download the OTA image data from a predetermined network address, verify it and then it will trigger the implant device OTA update by pushing the image data through the implant device OTA interface. In embodiments, after a successful OTA update installation, the implant device may restart and use the new software version.

In embodiments, the Recording Control Command may be a request to start or suspend the recording on the implant device. A pseudocode example of a Recording Control Command is shown in FIG. 70. In embodiments, when executing this command, the Gateway may send the request to start or suspend recording or neuronal activity to the controlled implant device.

In embodiments, the Status Command may be a request to update the implant device Status on the Cloud. A pseudocode example of a Status Command is shown in FIG. 71. In embodiments, when executing this command, the Gateway may request the status information from the implant device and push the status information to the Cloud.

In embodiments, the Gateway Configuration Command may include information about the new configuration that needs to be set on the Gateway. A pseudocode example of a command message is shown in FIG. 72. In embodiments, the configuration parameters may include information such as Local Wi-Fi network credentials, Cloud host network address, local administration credentials, network addresses of connected implant devices, implant device heartbeat checking interval, etc.

In embodiments, the Gateway may have a predefined buffer for recording data from the implant device. In embodiments, when this buffer is full, the recordings may be pushed to the Cloud. If real time data recording and streaming to the Cloud is needed, this buffer may be disabled or it may have a smaller size.

In embodiments, the Gateway OTA Command may include information about a new version of software to be installed on the Gateway. A pseudocode example of a command message is shown in FIG. 73. In embodiments, when executing this command, the Gateway may download the OTA image data from a predetermined network address, verify it, and then trigger the OTA update. In embodiments, after a successful OTA update installation, the Gateway may restart and use the new software version.

In embodiments, for each executed command, the Gateway may publish the status of execution back to the requestor of that command. In embodiments, when a command is added to the commands Queue, it will have an execution timestamp deadline. If the command is not taken from the Queue by any executor before the timestamp expires, the command may be marked with status “failed to execute” and the requestor may be informed about this failure. In embodiments, each command may be executed only once, irrespective of the result. The requester may decide to re-trigger the command in case of error, but this may be recognized as a new command. In embodiments, the commands may not contain any information related to the patient on which the implant device is applied. In embodiments, the commands may be executed only by the Gateway which controls the target implant device. In embodiments, the commands may be sent to Gateway over a secure channel. In embodiments, the system may guarantee the delivery of the commands to the Gateway component, where they may be executed. In case of error, the requestor of the command may be notified about the failure.

Data Service. In embodiments, Data Processing Service 6408 may be responsible for collecting the implant device data, decompressing the data (if need be), and storing the data for later use. In embodiments, there may be a large number of implant devices, which may send their data to the Cloud. Thus, on the Cloud, there may be a need for high scalability in recording this data and also there may be a demand to store a large amount of data. In embodiments, different technologies may support this. For example, the Publish/Subscribe Paradigm may enable the constant increase of implant devices and high parallelism of incoming data. In embodiments, the implant devices may act as data publishers while the Cloud that processes the data may act as a subscriber.

In embodiments, Data Service 6408 may be implemented as a microservice and may be deployed on multiple nodes on cloud. In embodiments, the Gateway may automatically upload the incoming data from the implant device to the Cloud. In embodiments, the Gateway may automatically register itself as a data publisher when one of the connected implant devices is starting to stream data. In embodiments, the communication channel between the Gateway and the Cloud may guarantee the delivery of the data. In case of connection errors, connection interruptions, lost packets, etc., the Gateway may be notified about the failure so that it can schedule a retry request. In embodiments, only a registered Gateway may stream data to the Cloud. Registered Gateways are those for which the provisioning step was executed and they have exchanged the encryption keys with the Cloud. In embodiments, the gateway and the Cloud may be connected over a secured channel. The messages transferred over this channel may be encrypted. The data streaming channel may be compliant with the existing medical standards.

In embodiments, for each channel, the implant device may record the specific value at a given time. The time of recording, reading value and recording type may be grouped together and may be streamed to the Cloud via the connected Gateway.

In embodiments, the data pushed from the Gateway to the Cloud may be time series data and may have a message structure similar to the example shown in FIG. 74. In this example, the message may include a plurality of floating point values, which may, for example, represent the data recorded from all active channels at a given timestamp, in which case, the order in the array may be fixed and may follow the physical tiles and channels numbering. As another example, the values may represent all data recorded from all active channels over a large interval of time. In embodiments, for each recorded channel the values may contain a timestamp=timestamp+blockIndex*readingInterval.

In embodiments, the data coming from the implant device may be encoded/compressed. Accordingly, when it arrives on the Cloud, the data may be reconstructed by applying a decoding/decompressing process. This process may include the entire pipeline of encoding/compression algorithms used at the implant device level while reading, processing, and sending data to the Gateway.

In embodiments, implant device data may be saved on the Cloud on a persistence layer in order to allow later-on batch processing and data retrieval. Any persistence technology may be used that provides the capability to handle the data volume. In embodiments, the data volume may be quite high. For example, an implant device may output up to 4 Mb/s. Assuming a full 24 hours recording, and 1000 implant devices, a data volume up to 432 TB per day may be produced.

Further, the persistence technology may provide the capability for data saving and retrieval to be as near to real time as possible. The high volume of data may generate big storage costs and also could increase the processing power needed for fast retrieval of the stored data.

In embodiments, to reduce the volume of data and to optimize the data retrieval speed, the persistence layer may support Backup Policies—based on predefined rules, the data that matches these rules may be backed up automatically, and Eviction policies—based on predefined rules, the data that matches these rules may be removed from the persistent storage.

In embodiments, Data Service 6408 may expose a data retrieval API that may be used by other Cloud services. This API may support data retrieval by using different filtering conditions. In embodiments, using this API and the filters, UI widgets, ML models, and data exporters may retrieve and use the data stored on the persistence layer. In embodiments, the interaction shall be performed through REST or QL filters.

In embodiments, after decoding and decompression, the implant device streamed data may be exposed to other components as a real time data stream, for example, for real time data visualization.

In embodiments, the incoming data from implant devices may not contain any information related to the patient. In embodiments, the Cloud may store the relation between the patients and implant device data, but this should be available only for Authorized User Roles and Authorized Operation Types. For example, researchers may have access only to anonymized data. In embodiments, practitioners may have access to patient private data only for the patients that are under their supervision.

In embodiments, in order to support high scalability during data ingestion, the data processing service may be deployed in a cluster computing environment. Each data stream event may be processed by a single cluster node. An example of an architecture 7500 for data ingestion and data processing is shown in FIG. 75. In this example, technologies that may be included may ease the implementation of the functional and nonfunctional requirements of the Data Processing Service. It is to be noted that although specific technologies are described in this example, one of ordinary skill in the art would recognize that other technologies that provide similar or equivalent functionality may be used instead, or in addition to, the described technologies.

For example, APACHE KAFKA™ 7502 may be used for data streaming and ingestion. It may be used for building real-time data pipelines and streaming apps. KAFKA™ is horizontally scalable, fault-tolerant, and very fast, being used in production by large companies. In embodiments, the data coming from implant devices may be distributed for processing to Cloud Data Processing Service 7504, which may be deployed in several nodes on the Cloud. KAFKA™ may also provide an easy method for starting/stopping the KAFKA™ Processors (the Cloud Data Processing Service 7504). In embodiments, APACHE KAFKA™ Security with its flavors TLS™, KERBEROS™, and SASL™ may help in implementing a highly secure data transfer and consumption mechanism.

In embodiments, APACHE KAFKA™ Streams 7506 may ease the integration of Gateway and Data Processing Service in the KAFKA™ Ecosystem.

In embodiments, APACHE BEAM™ may unify the access for both streaming data and batch processed data. It may be used by the real time data integrators to visualize and process the real time data content.

In embodiments, a high volume of predicted data and data upload and retrieval may be handled by a Time Series database Examples of such technologies may include OPENTSDB™—A Distributed, Scalable Monitoring System, TIMESCALE™—an Open-Source Time-Series SQL Database Optimized for Fast Ingest, Complex Queries and Scale, BIGQUERY™—Analytics Data Warehouse, HBASEυ, HDF5™, and ELASTICSEARCH™, which may be used as second index to retrieve data based on different filtering options.

In embodiments, add-on programs, such as GEPPETTO™ UI widgets may be used for visualizing neuronal activities. Further, KIBANA™ is a charting library that may be used on top of ELASTICSEARCH™ for drawing all types of graphics: bar charts, pie charts, time series charts etc.

Processing Pipelines. In embodiments, to give doctors and researchers the ability to manipulate the data and apply various algorithms to classify patient data, recognize patterns, recommend treatment, and do any types of processing, the Cloud component may support pipelines. In embodiments, the pipelines may include separate blocks, which may determine what data to process and what code to run over it. Each block may be configured individually. For example, the configuration may be done via a Drag and Drop UI or via a coding interface.

In embodiments, there may be different kinds of pipelines, for different use cases. For example, a real-time processing pipeline may be used by doctors to treat patients. This pipeline may have low latency and may not need high throughput. Another example is a batch processing pipeline, which may be used by researchers who want to train new models. This pipeline may have very high throughput, but the latency requirements may not be high. Another example is an automatic pipeline based on a central schema, which may be used for aggregating and analyzing data from different sources, and for scheduling automatic training and processing in the entire system.

Real-time Processing. In embodiments, to enable the system to respond quickly to incoming data from the implant devices, real time processing may be provided. This means that each data point (for example, electrical measurement taken by the implant device) is processed as soon as it arrives into the cloud database. An example of an API that may be used to specify the input for real time processing is shown in FIG. 76.

In embodiments, after specifying inputs, other kinds of operators may be applied to the data, element-wise, such as band pass filters, smoothing, and dimensionality reduction such as ICA or PCA. An example of an API that may be used to specify the pre-processing for real time processing is shown in FIG. 77.

In embodiments, for real time processing, existing machine learning models may be applied to the data in order to obtain inferences about the patient. These machine learning models may exist in a central repository. These models may be annotated with information about what kind of diseases they apply to and what conditions have they been tested in (such as location of implant devices). An example of an API that may be used to specify the machine learning processing for real time processing is shown in FIG. 78.

In embodiments, after all the processing has been done, the result may be output. This may mean either saving to disk, in a patient's file for example, or shown in a visualization, so that a user may understand what is going in the patient's brain in real time, or it may be used to send information to the implant device about what kind of neural stimulation commands to give. An example of an API that may be used to specify the output for real time processing is shown in FIGS. 79a and 79b.

Batch Processing. In embodiments, researchers may train algorithms over the data of many patients. These algorithms may take a long time to train, so there are few latency requirements in this case, but they need to be able to process a large amount of data, processing gigabytes of data every second.

In embodiments, as input, the researchers may select data belonging to only some patients, according to various criteria (such as having a certain age, or a certain disease, etc.). The output of this pipeline may be the resulting trained models, along with statistics about how well they performed (accuracy, loss, etc.). An example of an API that may be used to specify the input for batch processing is shown in FIG. 80.

In embodiments, the preprocessing blocks for the batch pipelines may be similar to the Real Time Processing Blocks, and these functions may be accessed using a similar API.

In embodiments, for batch processing, the researchers may have the option to use existing machine learning models or they may train new models which may then be saved into a central repository. These models may be annotated with information about what kind of diseases they apply to and where the data for them has been obtained (such as location of implant devices). For existing models, similar processing blocks and API may be used as for the Real Time Processing. For training new models, an example of an API that may be used to specify the machine learning for training new models for batch processing is shown in FIG. 81.

Custom Blocks. In embodiments, researchers may have the ability to run custom blocks where they can run any code they want. These custom blocks may have access to standard machine learning libraries and servers such as MATLAB™, TENSORFLOW™, SCIKIT-LEARN™etc. An example of an API that may be used to specify the custom blocks for processing is shown in FIG. 82.

In embodiments, when the batch processing has been completed, the resulting model may be written to disk. At the same time, during training, a summary of the progress of the model training may be saved. An example of an API that may be used for output from batch processing is shown in FIG. 83.

Automatic Pipeline. An exemplary block diagram of an automatic pipeline 8400, which may be used for aggregating and analyzing data from different sources, and for scheduling automatic training and processing in the entire system, is shown in FIG. 84. Pipeline 8400 may provide a way of joining different fields of expertise in a common collaboration environment. Each researcher may define his own experiments/tests that may be linked in a common workflow. The output of one Module (research) can trigger (automatically) a Module prepared by another researcher. All Modules may be versioned and may be easily reproduced by any team member.

For example, data to be utilized may include data from sources such as handwritten notes, MRI data, EKG, data EEG data, data from new medical devices, scans, wearables (24×7), data from smartphones—audio, video, motion, game/response, data from prosthetics, implants, the Internet of Things (IoT), etc. Such data may be collected from, for example, patient interviews where patients perform tests while multitasking (Daily Living Activities), from analysis of audio and written notes (patient and doctor), from Linguistic analysis such as LXIO and MSI, emotional states, Sentic and sentiment data, cognitive analysis data (Past/future), from content and context—subtle delays, tremor, repetition, etc., and from analysis of sensor and video data including synchronous multimodal response to stimulus/testing. Data collection tactics may include a clinical-use multimodal network of sensors, motion detection—wristband, ankle band, EEG-earbud or non-invasive wearable, Stethoscope—EKG, audio, video, Bluetooth, reaction—smartphone, tablet, Brain Code Collection System (BCCS) including a network of sensors+backend cloud. Such sensors may be wireless and synchronous.

Collaboration is only meaningful with a general understanding of each other, this applies also for any process run through the pipeline. In embodiments, the core of Pipeline 8400 may be the Generic Schema (GS) 8402 that may be used to map all the different data elements used by the different Modules. GS 8402 may be seen as the common language (describing data) used by each of the Modules even when using different programming languages. Furthermore GS 8402 may be heavily used by the Reporting layer that reports and analyses results across all modules.

Modules 8404, also shown in FIG. 85. In embodiments, modules may be autonomous processes that may include Data Input 8502—one or more Data sets/sources, Transformation 8504-code & scripts needed to do the transformation on the input, and Data Output 8506—one or more result sets. In embodiments, each module may be run in the cloud and may launch spot instances. In embodiments, each module may accept as input any data formats. In embodiments, code used in Transformation 8504 may be versioned using a version management system. In embodiments, rolling forward and backward may be possible with the same data sets.

Cascading Modules—8406 in FIG. 84, also shown in FIG. 86. Each Module may have Data Inputs that may be of any commonly used file format or online stored data set. Alternatively, the Input 8602 of a Module may be defined as the Output 8604 from another Module. In embodiments, this feature may be used to define Cascading Modules 8406 (workflows) that perform their tasks based on other Modules. Monitoring of these flows may be done in a Console (start, end, duration).

Pipeline —8408 in FIG. 84, also shown in FIG. 87. In embodiments, the orchestration of all modules may be done in Pipeline 8408. By configuring each pipeline, one may define flows that take results from each of the different fields (electroencephalogram (EEG), local field potential (LFP) measurements, event-related potential (ERP) measurements, positron emission tomography (PET), computed tomography (CT), magnetic resonance imaging (MRI) etc.) and make coherent analyses. The Generic Schema (8402 in FIG. 84) may ensure the results are easy to understand and correlate.

Machine Learning (ML) Toolbox 8800, shown in FIG. 88. In embodiments, the toolbox may include layers such as Machine Learning Models for Signal Processing 8802 and for Image Processing 8804, Machine Learning Frameworks 8806, Data, and Software Stacks 8808 for Data Analysis, Data Processing, Cloud Computation, and Optimization Approaches 8810. Examples of Machine Learning Models for Signal Processing are shown in block 8802, and examples of Machine Learning Models for Image Processing are shown in block 8804. An example of a processing flow 8812 is also shown. Such processing flows may be customized depending on the needs of the task at hand.

In embodiments, some of the machine learning models may be general, applicable to all brain recording data. Examples of these may be Linear Discriminant Analysis and Sparse Logistic Regression. In embodiments, there may also be machine learning models which are targeted for a specific disease, such as Alzheimer's disease and Parkinson's disease.

In the case of Parkinson's disease, the machine learning models may be trained to recognize when the patient is having motor problems, either with bradykinesia or excessive tremors. When detecting these mental states, a signal would be sent to start activating neurons in the appropriate region, in order to help alleviate the symptoms.

In the case of Alzheimer's disease, the machine learning models may be used to recognize when a patient has problems recalling already learned concepts and stimulation may be applied to help in memory improvements.

The cloud system may also implement the Fundamental Code Unit framework to analyze and correlate all the data of a patient starting from low-level neurotransmitter levels and neural spiking data, to high level behavioral data such as language and gait analysis.

Data Processing. In embodiments, there may be many approaches for data processing and pre-processing. The methods used for this phase may depend on the type and state of the data that is to be processed and on the specifics of the task the system needs to solve. Examples of such processing may include Normalization, Standardization, Mean Removal, Filtering (ex. High/Low Pass), Artifact Rejection, Epoch Selection, Feature Extraction, Data Cleaning, Data Transformation, Image Segmentation, Image Augmentation, Image Enhancement etc.

Optimization Techniques. In embodiments, each model may have its own specific optimization aspects that may be handled. Examples of such optimization may include Optimizing Hyperparameters, such as Hill Climbing (Random Restart), Simulated Annealing, Genetic Algorithms, MIMIC, MCMC, Expectation Maximization, and Grid Search, as well as Gradient Descent Optimization, Stochastic Gradient Descent Optimization, Adaboost, Memento etc. In embodiments, these optimization techniques may be modified or customized. Likewise, other optimization techniques may be utilized.

User Interface. In embodiments, the Cloud User Interface (UI) may have, for example, three different types of users, each of which may have different capabilities.

Patients. In embodiments, the UI for the patients may be focused on data visualization. They may be able to see real time activity as it comes in from the implant device.

Patients may also be able to select from a list of stimulation commands that were prescribed by the doctor. These commands may be either based on their current activity (sleep, walk, etc.) or based on their physiological state (tremors, inability to focus, etc.). Patients may also be able to annotate certain time segments with activities they were involved in during that time span to indicate, for example, when they were doing physical activities, mental tasks, etc.

Doctors. In embodiments, doctors may be able to access individual patient data. For each patient, they may have the option to apply different predefined machine learning models-presented as software-based prescriptions—in order to determine the best treatment going forward. Doctors may be able to configure the implant device, based on the output of the previous models. They may be able to set different modes of operation for the implant device, and change its recording/stimulation parameters. They may also be able to visualize the data of the patient in different ways, and flag certain patients for detailed analysis from neuroscientists.

Researchers. In embodiments, researchers may compose pipelines to process the data from many patients. An example of a general description of such a pipeline 8900 is shown in FIG. 89. In this example, pipeline 8900 may include reading patient data from a database 8902, processing the data 8904, training a machine learning classifier model 8906, validating the results 8908, and saving the trained model to storage 8910, such as disk.

Visualization Interface. In embodiments, the system may interface with tools such as EEGLAB™, which is a widely used neuroscience package for MATLAB™ or GEPETTO™ which can be used to visualize neurons, in order to provide Visualization Interfaces with which researchers are already familiar. In embodiments, examples of visualization methods may include Scalp Maps, ERP Images, Line Charts, Neuron Visualizations, Data Statistics, etc.

Security. Given the medical nature of the data handled by the system, great care must be taken to avoid any unauthorized access to the data or any commands sent by unauthorized agents. Accordingly, embodiments may provide secure communications, secure streaming, secure access, and secure storage. For example, providing secure communications may include ensuring that all the RPCs (Remote Procedure Calls) issued between the various microservices that make up the system are encrypted using the latest SSL encryption standards. In embodiments, data that is streamed from the Gateway may also be encrypted, to prevent tampering and snooping. In embodiments, secure access may be provided by an Identity and Access Management layer, which may give permissions to each actor to access and execute only user specific data and commands. For example, patients should be able to view only their own data and send to the implant device commands that have been authorized by a doctor, doctors should be able to only view the full data of their patients, use pretrained models to prescribe new software-based treatments for their patients and send commands to their patients' implant devices. In embodiments, researchers should have access only to anonymized patient data that they can use for deriving new scientific insights using the AI Research Interface provided in the Cloud environment. In embodiments, to prevent unauthorized physical access in data centers and provide secure storage, the data may be stored with encryption.

Consistency & Durability Requirements. In embodiments, there are a variety of aspects that may be considered in terms of system availability, consistency, and fault tolerance. For example, issues such as location, data consistency, maintenance, and backups may be considered.

Location. In embodiments, the cloud servers may be placed in a single region or in multiple regions. Multiple regions may mean higher availability due to outages that take out a single region, but comes at higher cost and higher system architecture complexity.

Data consistency. In embodiments, data may be stored in multiple copies to reduce the chance of one outage leading to the deletion of all the data. In embodiments, the choice may be between consistent availability, meaning that all the data is the same all the time and everywhere, at the cost of higher latency, or eventual availability, which means that depending on where the data is read from, different information might be returned.

Maintenance and DevOps. In embodiments, there may be a tradeoff to be made between running the system on premises or on public cloud providers such as AMAZON WEB SERVICES, GOOGLE CLOUD PLATFORM™ or AZURE™. This is because of different costs, maintenance work and infrastructure development. Considering the requirements for scaling up, public clouds may become cost-prohibitive, so they may be replaced with private hosted clouds, such as KUBERNETES™, or specialized clouds.

Backups. In embodiments, in order to ensure that data is not lost in case of system failure, regular backups may be done. They may happen at several levels. For example, data may be stored redundantly at the datacenter levels—to prevent loss due to individual machine failures. Likewise, data may be regularly copied to an offsite storage—to protect against geographic catastrophes.

An example of a process 9000, which is of a portion of a process of fabrication of CNT implant devices, is shown in FIG. 90. In this example, a microelectrode array of connections between electronic readouts and in-vivo human neural tissue may be fabricated. Using electroplating as a deposition technique, a CNT-based microelectrode array may be formed through a 1-mm thick micro-channel glass array (MGA) substrate. In an embodiment, the electrode arrays may have CNT contacts on the front side, and metal contacts on the back. In an embodiment the electrode arrays may have metal contacts on both sides.

Process 9000 may begin with 9002, in which an MGA substrate may be formed. At 9004, metal electrodes may be formed on the backside of the MGA substrate. At 9006, gold micro wires may be electrodeposited on the metal electrodes in the micro channels of the MGA substrate. At 9008, the topside of the MGA substrate may be etched to expose the gold micro wires. At 9010, the CNT material may be electrodeposited onto the exposed gold micro wires. At 9012, the backside of the MGA substrate may be etched to expose the backside gold micro wires.

An example of a process 9100, which is of a portion of a process of fabrication of CNT implant devices, is shown in FIG. 91. In this example, the MGA/CNT-based microelectrodes may be hybridized to an electrical readout chip providing for a parallel neural-electronic interface to the brain. Process 9100 may begin with 9102, in which an appropriate readout chip design may be selected. At 9104, metal bumps, such as indium, may be deposited on the contacts of the readout chip. At 9106, the micro wires that were exposed on the backside at 9012 in FIG. 90 may be pressed onto the metal bumps, creating electrical contact with the readout chip.

An example of a recording and stimulation signal and data flow on an implant device is shown in FIG. 92.

An example of a recording and stimulation signal and data flow on the Gateway and Cloud is shown in FIG. 93.

An exemplary block diagram of an embodiment of an implant device electrical system 9400 is shown in FIG. 94. In this example, system 9400 includes Vertically Aligned NanoTube Array (VANTA) 9402, cable 9404, analog multiplexers 9406, gain block 9408, ADC 9410, DAC 9412, control/processing circuitry 9414, and Wi-Fi communication circuitry 9416.

In embodiments, VANTA 9402 may include an array of vertically aligned nanotubes, as discussed above. Cable 9404 may connect VANTA 9402 to electronic circuitry, such as multiplexers 9406. In embodiments, cable 9404 may include a double layer flex cable, to connect the VANTA to the Analogue Front-end. Flex circuits offer the same advantages of a printed circuit board—repeatability, reliability, and high density—but with the added features of flexibility and vibration resistance.

In embodiments, the amplitudes of the analog signals may be adjusted by gain block 9408, which may include a plurality of amplifiers, one for each ADC. In embodiments, a plurality of ADCs 9410 may be multiplexed to a plurality of signals from VANTA 9402 by multiplexers 9406. The switching speed of multiplexers 9406 may be faster than the sampling frequency of ADCs 9410 by the number of the probes divided by the number of ADCs. Accordingly, in embodiments, the multiplexing frequency may be given by Fmux=CEIL(128 probes/16 ADCs)*3 kHz=24 kHz. The switching is fast enough so that the time taken to do a full scan of all the multiplexed channels would not significantly affect the measurement of the channels.

In embodiments, the ADC conversion may be triggered by the measured potential crossing a set threshold. As soon as the triggered ADC conversion starts, the adjacent ADCs may also be triggered.

In embodiments, in order to increase the Signal to Noise Ratio (SNR) and acquire position data of action potential source, several ADC measurements may be taken simultaneously, in a grid formation. The grid dimensions may be dependent on probe spatial density. An example of a 4×4 probe multiplexer distribution is presented in FIG. 95. All the squares with the same number represent probes which share the same Amplifier and ADC through a multiplexer. The probes may be connected to multiplexers in such a way that, no matter which ADC is being triggered, no adjacent probe shall be multiplexed to the same channel.

After a 3×3 ADC grid is acquired (the grid containing the triggered channel and the surrounding 8 channels), the results may be processed by control/processing circuitry 9414. Control/processing circuitry 9414 may include a microcontroller or other computing device, as well as hardware processing functions, which may be implemented, for example, in an FPGA or ASIC. Such hardware processing may perform, for example, multiplication to increase SNR, weighting to accurately place the signal source, etc.

An exemplary embodiment of a portion of an implant device electrical system is shown in FIG. 95.

For example, as shown in FIG. 96, the action potential may fire in square 9602 with and may cross the set threshold. As a result, the corresponding ADC and all the adjacent ADCs 9604 may be triggered. Because the maximum length of an action potential is about 5 ms, all 9 ADCs may obtain samples for that time. The resulting data may be processed in control/processing circuitry 9414. For example, the signals may be multiplied to increase SNR. At the same time, based on the signal intensity, a point may be placed on the calculated position with the highest potential—spatial resolution depends on the number of channels sampled.

An example of the triggering of the first ADC and the quantization of the action potential is illustrated in FIG. 97 for a 3 kHz sampling rate. For a 5 ms long spike, the curve may be described by 16 points and model-based reconstruction of the signal may be used on the recorded data. In embodiments, the reading sampling rate may be increased, up to, for example, about 96 kHz, with increased power consumption.

An exemplary block diagram of multiplexer connections 9800 for two pairs of differential probes 9802, 9804 is shown in FIG. 98. Notice that the positive and negative probes are each connected to different multiplexers 9806, 9808 for simultaneous availability. As the DAC is enabled, the ADC is disabled for the same pair, allowing the reuse of the same multiplexer.

In embodiments, for recording, the signal from the multiplexer may be amplified using a Gain Block 9900, such as the example shown in FIG. 99, before being input to the ADC sampling unit. In embodiments, the First Amplifier Stage may include a differential input fixed gain instrumentation amplifier 9902. This design, while not adding much complexity, may be characterized by a low noise figure and a high common mode rejection ratio. It also doubles as an input driver with a very high input impedance, reducing load on the signal. In embodiments, amplifier stage 9902 may be followed by a switched capacitor bandpass filter of, for example, 3 kHz, to filter out the MUX switching noise. In embodiments, the Second Amplifier Stage may include a variable gain amplifier 9906 having a gain range of, for example, 1 to 128. The gain of amplifier 9906 may be programmable using, for example, a Gain and Clamp Adjust DAC Block, which may correct for clipping caused by probe-neuron distance variation.

An exemplary block diagram of a Gain Block 10000 is shown in FIG. 100. In this example, Gain Block 10000 may include a differential two stage variable gain amplifier 10002, such as the VCA2617 from TEXAS INSTRUMENTS®, low pass anti-aliasing filter 10004 having a bandwidth of, for example, 3 kHz, and a gain and claim adjustment block 10006, such as the AD7398/AD7399 from ANALOG DEVICES®. In this example, amplifier 10002 may be continuously variable, voltage-controlled gain amplifier. Adjustment block 10006 may accept digital data to control DACs and output voltages to control the gain and clamping of amplifier 10002. Low pass filter 10004 may, for example, be implemented using passive components and may be used to restrict the bandwidth of signal before being sampled by the ADCs.

In embodiments, in order to measure a total of 128 differential probes, a compromise may be found between a high enough number of simultaneously sampled channels, for good signal characteristic, and a low number of ADCs, for space saving considerations. In embodiments, a 3×3 grid may be used, requiring a total of 9 triggered ADCs.

In embodiments, an ADC 10100, an example of which is shown in FIG. 101, such as the ADS1278 from TEXAS INSTRUMENTS®, may be used. In this embodiment, each ADC device may have 8 simultaneous sampling channels, thus, two ADS1278 devices may be used for a total of 16 simultaneous measurements. After multiplexing each ADC channel to 8 differential probes, the total 128 necessary measurement channels may be obtained. It is to be noted that the ADS1278 is a high precision 24-bit ADC with a high-power consumption. Given that the signals are repetitive in nature, embodiments may only need 10 bits of ADC precision for the encoding of the action potential signal. Accordingly, other ADCs having lower precision and lower power consumption may advantageously be utilized in embodiments.

In embodiments, DAC Block circuitry 10200, an example of which is shown in FIG. 102, such as the LTC1450/LTC1450L from ANALOG DEVICES®, may be used for electrical stimulation of the neuronal tissue through the CNTs. DAC Block 10200 may include an array of high resolution DACs. The stimulation circuit may be able to generate multiple arbitrary waveforms. In embodiments, the DACs may interface with control/processing circuitry 9414 using a parallel or serial architecture in which all DACs are sharing the same data bus.

In embodiments, each DAC may have a Load Data Signal Line used for data output register update. The control/processing circuitry 9414 may load sample data into each DAC. After all the data has been uploaded, a single Load Data Line Toggle may set the analog output of the DAC at the desired values.

For example, consider 8 discrete signals having 256 samples stored as a matrix: stimulus_name[DAC_resolution][sample]. In this example, a write process may include loading a first sample of each stimulus into a corresponding DAC, toggling all Load Data Lines simultaneously and updating DAC output voltages, loading the next samples repeatedly until the stimulus signals have been generated, and setting the output channels to high impedance.

In embodiments, due to the quantization levels of the DAC, the output voltage may be affected by slight transitions. In order to clean up the signal, a low pass filter may be inserted at the DAC outputs.

In embodiments, operational modes for the Closed Loop Process may include Sequential Reading and Stimulation and Simultaneous Reading and Stimulation. The Sequential Reading and Stimulation mode may share the same Mux/Demux block between ADCs and DACs. This method may reduce design complexity, but cannot stimulate and read the neuronal activity in different locations of the tissue at precisely the same time.

The Simultaneous Reading and Stimulation mode may use a plurality of Mux/Demux blocks for ADCs and DACs. The high impedance of the ADC inputs and the Gain Block will not affect the stimulation. In embodiments, this architecture may stimulate the neuronal activity in a certain location and measure the response signal in an arbitrary location. There may be the need to set two different Mux/Demux addresses: one for stimulation and one for impulse response.

In embodiments, with use of the Multiplexing Pattern described above, the shortcomings of the first operation mode are alleviated, as there will be no two simultaneous writes in the same 4×4 cell.

In embodiments, control/processing circuitry 9414, shown in FIG. 94, may include a microcontroller or other computing device, as well as hardware processing functions, which may be implemented, for example, in an FPGA or ASIC. For example, in an FPGA implementation a SPARTAN-7® FPGA from XILINX® may be utilized. In another example, an IGLOO NANO® from MICROSEMI® may be used.

In embodiments, control/processing circuitry 9414 may perform data acquisition from the ADCs; separation of overlapped signals; action potential recognition and sorting including finding firing patterns, isolating signals from each other, and eliminating crosstalk temporally (time window cropping) and dimensionally (close signal multiplication); creating a perceived map of neurons based on signal strength and pattern recognition, thus further reducing necessary data throughput, and detecting higher-order features of the neural network.

In embodiments, control/processing circuitry 9414 may include a microcontroller or microprocessor for serialization, debugging, communication and control. For example, a single or multi-core CPU may be used. In embodiments, embedded memory, external memory, and peripherals may be located on the data bus and/or the instruction bus of these CPUs. An adequate address space, such as 4 GB, and functions such as DMA and built-in Wi-Fi may be utilized. Control/processing circuitry 9414 may be used for controlling the hardware components (MUX, ADC, DAC) and data transmission and acquisition rates.

Optical Recording & Stimulation. In embodiments, the range of radiation wavelengths for neuron stimulation may be between 380 nm and 470 nm, which may be obtained using one single LED by modulating the current characteristics. For example, a pixel density of 570 ppi (pixels per inch) for a 2×2 array (for color) will yield a pixel 22.3 microns wide. Depending on the pitch of the CNTs, the LEDs may be placed either in between the CNTs or right underneath them (the wires connected to the CNT may be run through the LED).

Optical Reading. In embodiments, if LEDs are used for optical stimulation, options for optical recording may include using the LEDs as radiation receptors to convert light into electric signals and using image sensors, such as CCD or CMOS image sensors. In embodiments, if LEDs are used as radiation receptors, the same device may be used both for optical stimulation and recording. In these embodiments, the recorded electric signal may be relatively weaker and noisier. This is an important drawback especially when the recorded signals have such small values. In embodiments, use of CCD or CMOS photodiodes may provide a stronger signal. In these embodiments, the optical reading and stimulation resolution may decrease due to the fact that these sensors have to be added in addition to the existing LEDs.

In embodiments, the circuitry may be in the form of a readout-integrated circuit (ROIC), which may be similar to or a modification of, for example, a solid-state imaging array. The ROIC may include a large array of “pixels”, each consisting of a photodiode, and small signal amplifier. In embodiments, the photodiode may be processed as a light emitting diode, and the input to the amplifier may be provided by the CNT connection to the neuron. In this manner, neurons may be stimulated optically, and interrogated electrically. The ROIC may include CCD or CMOS photodiodes or other imaging cells, to receive optical signals, electrical receiving circuitry, to receive electrical signals, light outputting circuitry, such as LED or lasers, to output optical signals, and electrical transmitting circuitry, to transmit electrical signals.

In embodiments, the light sources may be placed at the base of the CNTs, rather than using optic fibers. In these embodiments, the light does not have to be transported from the light sources to the recording site and back using an optical circuit. Exactly how many neurons may be optically reached depends on the distance between the neuronal tissue and the CNT board which in turn depends on the length of the CNTs. In these embodiments, a plastic magnifier on the LED may be used to focus the light emission. But considering the width of one LED is about 23 microns, this would be a challenging solution in terms of manufacturing.

In embodiments, optical fibers may be used to take the emitted wave from the light source to the tissue. For example, for fiber optics with glass fibers, light may be used with wavelengths longer than visible light, typically around 850, 1300 and 1550 nm. The reason these wavelengths are preferred is that attenuation in the fibers is smaller for these wavelengths. As shown in FIG. 103, scattering effects are lower as the wavelength increases, and absorption occurs in in several specific wavelengths (called water bands), due to the absorption by minute amounts of water vapor in the glass. However, these wavelengths may be significantly larger than what it is needed for neural stimulation (380 to 470 nm). In embodiments, plastic optical fibers may be used.

An exemplary block diagram of a computing device 10400, which may be included in control/processing circuitry 9414, shown in FIG. 8, in which processes involved in the embodiments described herein may be implemented, is shown in FIG. 104. Computing device 10400 may be a programmed general-purpose computer system, such as an embedded processor, microcontroller, system on a chip, microprocessor, smartphone, tablet, or other mobile computing device, personal computer, workstation, server system, and minicomputer or mainframe computer. Computing device 10400 may include one or more processors (CPUs) 10402A-10402N, input/output circuitry 10404, network adapter 10406, and memory 10408. CPUs 10402A-10402N execute program instructions in order to carry out the functions of the present invention. Typically, CPUs 10402A-10402N are one or more microprocessors, such as an INTEL PENTIUM® processor. FIG. 104 illustrates an embodiment in which computing device 10400 is implemented as a single multi-processor computer system, in which multiple processors 10402A-10402N share system resources, such as memory 10408, input/output circuitry 10404, and network adapter 10406. However, the present invention also contemplates embodiments in which computing device 10400 is implemented as a plurality of networked computer systems, which may be single-processor computer systems, multi-processor computer systems, or a mix thereof.

Input/output circuitry 10404 provides the capability to input data to, or output data from, computing device 10400. For example, input/output circuitry may include input devices, such as keyboards, mice, touchpads, trackballs, scanners, etc., output devices, such as video adapters, monitors, printers, etc., and input/output devices, such as, modems, etc. Network adapter 10406 interfaces device 10400 with a network 10410. Network 10410 may be any public or proprietary LAN or WAN, including, but not limited to the Internet.

Memory 10408 stores program instructions that are executed by, and data that are used and processed by, CPU 10402 to perform the functions of computing device 10400. Memory 10408 may include, for example, electronic memory devices, such as random-access memory (RAM), read-only memory (ROM), programmable read-only memory (PROM), electrically erasable programmable read-only memory (EEPROM), flash memory, etc., and electro-mechanical memory, such as magnetic disk drives, tape drives, optical disk drives, etc., which may use an integrated drive electronics (IDE) interface, or a variation or enhancement thereof, such as enhanced IDE (EIDE) or ultra-direct memory access (UDMA), or a small computer system interface (SCSI) based interface, or a variation or enhancement thereof, such as fast-SCSI, wide-SCSI, fast and wide-SCSI, etc., or Serial Advanced Technology Attachment (SATA), or a variation or enhancement thereof, or a fiber channel-arbitrated loop (FC-AL) interface.

The contents of memory 10408 may vary depending upon the function that computing device 10400 is programmed to perform. For example, as shown in FIG. 1, computing devices may perform a variety of roles in the system, method, and computer program product described herein. For example, computing devices may perform one or more roles as end devices, gateways/base stations, application provider servers, and network servers. In the example shown in FIG. 104, exemplary memory contents are shown representing routines and data for all of these roles. However, one of skill in the art would recognize that these routines, along with the memory contents related to those routines, may not typically be included on one system or device, but rather are typically distributed among a plurality of systems or devices, based on well-known engineering considerations. The present invention contemplates any and all such arrangements.

In the example shown in FIG. 104, memory 10408 may include sensor data capture routines 10412, signal pre-processing routines 10414, signal processing routines 10416, machine learning routines 10418, output routines 10420, databases 10422, and operating system 10424. For example, sensor data capture routines 10412 may include routines that interact with one or more sensors, such as EEG sensors, and acquire data from the sensors for processing. Signal pre-processing routines 10414 may include routines to pre-process the received signal data, such as by performing band-pass filtering, artifact removal, finding common spatial patterns, segmentation, etc. Signal processing routines 10416 may include routines to process the pre-processed signal data, such as by performing time domain processing, such as spindle threshold processing, frequency domain processing, such as power spectrum processing, and time-frequency domain processing, such as wavelet analysis, etc. Machine learning routines 10418 may include routines to perform machine learning processing on the processed signal data. Databases 10422 may include databases that may be used by the processing routines. Operating system 10424 provides overall system functionality.

Embodiments of the present systems and methods may provide machine learning techniques that may address such shortcomings and provide improved performance and results. For example, embodiments may address issues in the context of, for example, natural language processing (NLP), in a multidisciplinary approach that aims to bridge the gap between statistical NLP and the many other disciplines necessary for understanding human language such as linguistics, commonsense reasoning, and affective computing. Embodiments may leverage both symbolic and subsymbolic methods as that use models such as semantic networks and conceptual dependency representations to encode meaning, as well as use deep neural networks and multiple kernel learning to infer syntactic patterns from data.

Embodiments may provide an intelligent adaptive system that combines input data types, processing history and objectives, research knowledge and situational context to determine what is the most appropriate mathematical model, choose the most appropriate computing infrastructure on which to perform learning, and propose the best solution for a given problem. Embodiments may have the capability to capture data on different input channels, perform data enhancement, use existing AI models, create others de novo and also finetune, validate, and combine them to create more powerful collections of models. Embodiments may use concepts from the critic-selector model of mind and from the brain pathology treatment approaches.

Embodiments may be used for different types of applications. For example, embodiments may be used for human-machine interaction problems due to their anthropomorphic and data-adaptive capabilities. Anthropomorphism refers to the capability of the system to react differently depending on the profile and preferences of the human with whom the machine interacts, and it is data-adaptive in the sense that it chooses the best fitting mathematical approach to the input data it receives from the human.

An exemplary block diagram of a system 10500 according to the present techniques is shown in FIG. 105. System 10500 may include, for example, three layers, Input Data Layer 10502, BrainOS Data Processing Layer 10504, and output data layer 10506. Input Data Layer 10502 may include data-capturing points from data channels 10508 associated with types of data: video, image, text, audio, etc., as well as meta world data 10510 and objective data 10512. The data channels layer may include several stages of data retrieval and manipulation, such as: identification of input points and types for each data channel, retrieval of data and data preprocessing, and data sampling techniques and storage.

BrainOS Data Processing Layer 10504 may include a model selector 10514 and a model repository 10516. Model selector 10514 identify a set of methods and operations from model repository 10516 to apply on the input data in relation to intelligence inferring and pattern determination. Such mechanisms may include the stages such as a Critic-Selector Mechanism, which may be based on combining input data types from data channels 10508, meta world data 10510, such as processing history, and objective data 10512, including research knowledge and situational context to determine what is the most appropriate Artificial Intelligence (AI) model for existing data and how the system should manage the processing resources, be it models or computing infrastructure. Such mechanisms may further include data processing using AWL algorithms in pipelines and a models training loop and transfer learning mechanism.

Output Data Layer 10506 may include the results of running the resulting model or ensemble of models on the automatically selected computing infrastructure.

Embodiments of the present systems and methods may operate on data channels, data processing methods and model selector components, and utilizes a repository of intelligent models (similar to the specific neural networks in the human brain). Embodiments may be underpinned by a complex qualifier-orchestrator meta-component, which is based on a critic-model selector component that performs automated determination of models to be employed for solving any given scenarios.

Embodiments may use available computing infrastructure as a set of resources that can be turned on and off through a critic-selector mechanism, much in the way the human mind seems to work. This principle can be applied at different layers, as described further below. The human brain uses different neuronal areas to process input data, depending on the receptor type. There are specific neural networks associated to different brain functions, as illustrated in FIG. 106.

Mimicking the brain, embodiments may feature a critic-selector mechanism (shown in FIG. 108). The critic-selector mechanism may process the problem description, recognize the problem type, and then activate the selector component. The selector may start up several sets of resources (models or combination of models), which were learned from experience as the most probable viable approaches for the given situation at hand.

Embodiments may feature multi-modal processing combining data, which maps to the human senses of vision, hearing, etc., and a multitude of “data senses”, meaning other cross-correlated data streams which can be mined for information.

The Brain Pathology Treatment Mimetic. The human brain, which has been referred to as a “three pound enigma,” is considered the grand research challenge of the 21st century. We understand the brain as a multidimensional, densely wired matter made of tens of billions of neurons, which interact at the millisecond timescale, connected by trillions of transmission points that generate complex output such as behavior and information processing. Neurons can send to and receive signals from up to 10⁵ synapses and can combine and process synaptic inputs to implement a rich repertoire of operations that process information.

Parkinson's Disease Example. Neurodegeneration is a progressive loss of neuron function or structure, including death of neurons, which occurs at many different levels of neuronal circuitry. One of the most devastating and currently incurable neurodegenerative diseases (NDD) is Parkinson's Disease (PD).

PD is a chronic, progressive NDD usually found in patients over 50 years of age. PD is the most common form of Parkinsonism, a group of conditions that share similar symptoms. Symptoms and severity vary from patient to patient, making diagnosis difficult. The classic triad of symptoms comprise tremor at rest, muscle rigidity and bradykinesia (slowing of all movements, particularly walking). Postural instability, grossly impaired motor skills, and general lethargy are also common. These symptoms are caused by the death of neurons in the substantia nigra pars compacta in the midbrain that control movement by releasing dopamine into the striatum of the basal ganglia; dopamine is a neurotransmitter that modulates neural pathways to select appropriate movements for individual circumstances. Some studies have found that PD patients also exhibit abnormal production of the neurotransmitter norepinephrine. Norepinephrine may be linked to non-motor symptoms of PD including fatigue, irregular blood pressure, and anxiety.

Treatment Approaches. There currently exists no way to stop the progression of the disease, but it can be managed using mainly two kinds of interventions—Pharmaceutical treatment and Surgical treatment.

The most common pharmaceutical intervention relies on using levodopa (L-DOPA), which is converted to dopamine by the surviving neurons in order to compensate for the degeneration of the dopamine-producing cells. Although it is the most effective pharmaceutical treatment for PD to date, L-DOPA can have severe side effects such as dyskinesias and motor fluctuations. Among the dyskinesia adverse effects we can mention tics, writhing movements, dystonias, as well as periods of time when the medication has no effect. Moreover, patients can develop unresponsiveness to L-DOPA requiring increased doses over time, which can lead to more severe side effects.

A promising therapeutic approach free from the side effects of levodopa treatment is using implanted devices for neural modulation through electrophysiology or optogenetics.

The Neural Modulation Treatment Approach. Using electrophysiology and/or optogenetics the chemical behavior of the neurons may be controlled. Brain stimulation is more effective when it is applied in response to specific brain states, via, for example, Closed Loop Monitoring, as opposed to continuous, open loop stimulation. A conceptual sketch of a closed loop control system can be seen in FIG. 107. As shown in FIG. 107, a target input 10702 may be applied to an error component 10704, which may generate an error signal 10706 that may be input to controller 10708. Controller 10708 may generate a control input signal 10710 based on error signal 10706, which may be applied to system under control 10712. System 10712 may generate an output, which may be measured 10716 and a signal 10718 representing the measured output may be input to error component 10704.

Embodiments may provide closed-loop, activity-guided control of neural circuit dynamics using optical and electrical stimulation, while simultaneously factoring in observed dynamics in a principled way. This may provide a powerful strategy for causal investigation of neural circuitry. In particular, observing and feeding back the effects of circuit interventions on physiologically relevant timescales is valuable for directly testing whether inferred models of dynamics, connectivity, or causation is as accurate in vivo.

Embodiments may use an evaluation function to measure how well the model performs on the validation data. If the error is larger than the defined tolerance, the controller modifies the tested model architectures and then proceeds again with the evaluation step.

In embodiments, depending on the complexity of the model and the number of features the algorithm needs to search, the evaluation function can become more elaborate. If there are multiple features for which we want to optimize, a multi-parameter evaluation function can be used, for example a combination of multiple heuristic functions. Then, based on the feedback from all the heuristic functions, a decision can be made concerning how the set of model architectures can be improved.

There are many approaches to implement a closed loop control algorithm. The simplest one is an on/off algorithm, illustrated in the pseudocode sequence below for a neural modulation application.

```
List<Channels> channels_to_read;
List<Channels> channels_to_stimulate;
while (!stopped) {
  neuron_data = read_channels(channels_to_read);
  next_state = calculate_next_state(neuron_data);
  if (next_state < threshold) {
    duration = calculate_duration(neuron_data);
      apply_stimulation(channels_to_stimulate, duration);
  }}
```

Architecture. Embodiments may provide the capability to adapt learning modules and resources to a specific input problem so as to propose the best solution for a given problem formalization. An exemplary embodiment of an overall architecture of a system 10800 is shown in FIG. 108. As shown in FIG. 108, data sources 10802 may include sensors 10804, devices 10806, such as Internet of Things (IoT) devices, servers 10808, robots 10810, humans 10812, etc. Data from data sources 10802 may be input to system 10800 through an exposed API 10814, and may adhere to a given schema. Data from API 10814 may be input to problem formalization component 10816.

Problem Formalization. Problem formalization component 10816 may be the main entry point in the system 10800 flow, and may include components such as Data channels 10818, Meta-World information 10820, and Task Objective 10822. These 3 components may include the entire set of available information with regards to a given input problem.

Data channels 10818 may include the information about a problem. Meta-World information 10820 may include information about the real world context and specific descriptions of the variables available in the input dataset, while the Task Objective 10822 may describe the main purpose of the processing task, and its desired results.

For reasons of consistency, the input to Problem Formalization component 10816 may comply to a problem formalization schema or format, which can be exposed through an API for connecting system 10800 to any other machine or system. Likewise, the output from Problem Formalization component 10816 may comply to a defined schema or format. Hence, problem formalization component 10816 may also play the role of maintaining the problem's integrity and consistency, to provide for the proper functioning of the next modules in the pipeline of the system.

History Databases. The task of proposing an adaptive learning system for solution proposal in a dynamic environment is an elaborate undertaking, bringing us closer to the realms of human reasoning and understanding. It is clearly known that humans make use of complex and vast fields of knowledge and experiences when they are trying to search for solutions to even simple issues and obstacles in their daily lives. To mimic the extraordinary human cognitive ability, system 10800 may include at least two storage systems.

One storage system, History Storage Component 10824 may include experience acquired over the entire life of the system, in terms of encountered data sets, previous used resources (models) and achieved results. For example, History Storage Component 10824 may include storage of information 10826 relating to previous problems presented to system 10800 and information 10828 relating to previous approaches that were used to solve the previous problems and the results of such approaches. Such a memory resource may be valuable in situations in which the system is confronted with similar problems to those processed in the past, conferring to system 10800 the capability of a “reflex response” when the encountered problem formulation is already known.

As a second layer of history, the World Knowledge Component 10830 may include “common sense” knowledge of the world, spanning from general concepts to domain-specific ones. World Knowledge Component 10830 may include Domain Knowledge information 10832, which may include information for a diverse range of disciplines and areas in which the system may have expertise, and Integrated Research Experience information 10834, which may serve as a bridge between the real world's interdisciplinarity and the system's homogeneous structure. Integrated Research Experience information 10834 may include Stored Models 10836—resources discovered in the past and open for direct use without any property constraints and the more abstract Research Knowledge 10838—a vast field of information, parts of which could be applied to specific problem formulations, distinct problem solutions, or precise data sets. Such information may be obtained from public and proprietary sources, for example, from the Internet.

World knowledge component 10830 may include both code and ontologies and may be built using the available information on the web and in the online and offline academic contexts, by using an ensemble of Natural Language Processing (NLP) and web-crawling techniques.

Qualifier (Critic) Component 10840. The first processing phase may be accomplished using Qualifier (Critic) Component 10840, which may use Problem Formalization 10816 in the form of problem input 10841, Experience Information 10881 from history storage component 10824, and Filtered Knowledge 10880 from World knowledge component 10830 for processing such as:

Enhancing the data with any previously used data sets that match or complement the current input characteristics, in a Data Enhancer component 10842. Here the input data may be enhanced by parsing the entire available history of data sets (using their characteristics for finding their added value in enhancing the current data set) and exploring the correlations between vital concepts in the problem formulation.

Making qualifications and applying constraints on the problem at hand, for achieving an intermediate qualification result that can be used for narrowing down the reasoning search space in the next steps of the flow. This may be performed by Requirements Generator (Restrainer) component 10844. The Requirements Generator (Restrainer) component 10844 may apply “common sense” knowledge and may filter out data that is outside the current situational context.

Planner component 10846. The input data that Planner component 10846 works with may be the processed problem 10847 from Qualifier (Critic) Component 10840, which may include the problem formulation and the history of models used 10888 from history storage component 10824, together with their problem formulations and their results. Planner component 10846 may have the ability to determine the most appropriate processing flow for the current problem based on the World Knowledge, Objective, and the similarity of the current task with problems processed in the past.

As an example, for a problem of intent extraction from an image, planner component 10846 might prescribe the following steps:

1. Run captioning algorithms on the image to obtain a narrativization of the image

2. Run object detection and activity recognition on the image

3. Run an algorithm to obtain an ontology for the previously extracted concepts

4. Infer intent using all the previously obtained entities and ontologies

Planner component 10846 may be seen as a large bidirectional graph knowledge in which specific heuristic search algorithms may be run for the detection of the proper node sequences for a given task. For example, an embodiment may use multi-directional advanced versions of ALT search algorithm with Shortcuts and Reach.

An example of pseudocode for such an embodiment is shown in FIG. 109. Even the best search algorithms can be really expensive to run on large graphs. Table 1 below presents a summary of the running time for different classic search algorithms:

TABLE 1

| | | | | | Iterative | Bidirec- |
|---|---|---|---|---|---|---|
| | Breadth- | Uniform- | Depth- | Depth- | Deepen- | tional (if |
| Criterion | First | Cost | First | Limited | ing | applicable) |
| Complete? | Yes^{a} | Yes^{a,b} | No | No | Yes^{a} | Yes^{a,d} |
| Time | O(b^{d}) | O(b^{1+└C*/ϵ┘}) | O(b^{m}) | O(b^{l}) | O(b^{d}) | O(b^{d/2}) |
| Space | O(b^{d}) | O(b^{1+└C*/ϵ┘}) | O(bm) | O(bl) | O(bd) | O(b^{d/2}) |
| Optimal? | Yes^{c} | Yes | No | No | Yes^{c} | Yes^{c,d} |

Although heuristic search algorithms may improve over the above, still, in reality there is a large set of NP-Complete problems which are not solvable with such an approach. For these cases, embodiments may use optimization approaches using metropolis algorithms, such as simulated annealing, in the planning stage, for searching after improvements in a promising area which was already discovered using a lower level of heuristic search. Simulated Annealing, a version of stochastic hill climbing, uses a Monte Carlo based algorithm and a lowering temperature for converging to a local optimal. In sufficient time, this is expected to converge to a “canonical” distribution, such as:

νᵣ∝exp(−Eᵣ/kT),

where E is the potential energy of a system, calculated using the positions of the N particles:

$$E = \frac{1}{2} {\sum }_{i = 1}^{N} {\sum }_{j = 1}^{N} V ( {d}_{i j} ) , \\ i \neq j$$

An example of high-level pseudocode for simulated-annealing is presented below:
function SIMULATED-ANNEALING(problem, schedule) returns a solution state inputs: problem, a problem
   schedule, a mapping from time to “temperature”
current←MAKE-NODE(problem.INITIAL-STATE)
for t=1 to ∞ do
   T←schedule(t)
   if T=0 then return current
   next—a randomly selected successor of current
   [symbol] E←next. VALUE-current. VALUE
   if [symbol] E>0 then current←next
   else current←next only with probability

[symbol]

Parallel Executor 10848. Parallel Executor 10848 may perform the following:

Based on the plans 10850 made by planner component 10846, Parallel Executor 10848 may initiate different threads of execution for Selector component 10852 to generate appropriate models. Based on the models received from Selector 10852, such as selected models 10892 from criterion component 10874, which may be obtained by creation de novo or by a combination of existing models, Parallel Executor 10848 may split the processing tasks into multiple parallel threads. Based on the prepared processing threads, parallel executor 10848 may select the corresponding computing infrastructure in terms of hardware and software, such as clusters and virtual instances, etc.

In embodiments, Parallel Executor 10848 may instruct 10889 Infrastructor component 10875 to select the corresponding computing infrastructure in terms of hardware and software, such as clusters and virtual instances, etc. In embodiments, Solution Processor component 10856 may instruct 10890 Infrastructor component 10875 to select the corresponding computing infrastructure in terms of hardware and software, such as clusters and virtual instances, etc. For example, Infrastructor component 10875 may include or select frameworks 10876, containers 10877, graphic processing units 10878, etc., to perform the processing tasks, based on the determined amount and types of computing resources needed. In embodiments, Parallel Executor 10848 may instruct 10891 selector component 10858 to build or rebuild models.

Module Scheduler 10854. Module Scheduler 10854 may receive the stored module solution 10855, which may include the prepared threads, prepared by the Parallel Executor 10848, and makes a schedule for the solution's execution. This may include different resources at processed at the same time, from the network.

Solution Processor 10856. Solution Processor 10856 may receive the scheduled tasks or process modules 10857 and runs them, if needed in parallel, on the appropriate computing infrastructure.

In embodiments, Parallel Executor 10848, Module Scheduler 10854, Solution Processor 10856 may reflect at a higher level the already established and efficient approaches in terms of computer architecture (FIG. 110), and cloud computing (FIG. 111).

Selector component 10852. Selector component 10852 may prepare the appropriate model for the given problem formulation. To be able to deliver an appropriate model, approaches the Selector may use may include:

History Model Selector component 10858 may search for and select 10859 one or more appropriate models among previously used processed models stored in history storage component 10824. If the Selector component 10852 finds a good fit, then the model may be tuned 10860, and Model Processor component 10863 may train 10864 and evaluate 10865 the model.

Research Based Builder component 10861 may search 10862 the Research Knowledge, such as published models 10884 and published papers and public code implementations stored in World Knowledge Component 10830. If one or more good candidates are found, then the model(s) may be tuned, and Model Processor component 10863 may train 10864 and evaluate 10865 the model(s) and send the models for storage 10885 in online model repository 10886.

Model Designer component 10866 may build one or more new models from scratch after type 10867, morphology 10868, and parameters 10869 are determined. Subsequently the model may be tuned, and Model Processor component 10863 may train 10864 and evaluate 10865 the model(s).

From ensemble learning methods we know that a combination of lower accuracy models may perform better than a higher accuracy model due to overcoming bias. Therefore, before the Selector component 10852 adopts the solution model for the given problem formulation, Model Ensembler component 10870 may determine, using, for example, selected 10871 and trained heuristics 10872 and/or machine learning models, whether there is a combination of models that can outperform the selected model. If Selector component 10852 finds such a model combination, then the model solution may include an ensemble of models. At least one or more of History Model Selector component 10858, Research Based Builder component 10861, and Model Designer component 10866 may provide one or more models to be evaluated by Model Ensembler component 10870. The chosen model or ensemble of models may then be added to models stored in history storage component 10824, together with the problem formulation and obtained accuracy.

Any or all such approaches may be run in parallel, and each module may store the current best achieved models in Online Model Repository 10873. Criterion component 10874 may signal a stop processing event 10883 based on stop criteria 10887, for example, when a model that is adequate for the objective is found, or when one of the model selector components 10858, 10861 10866, 10870 should not be involved in searching anymore given the low probability of finding a proper solution using that approach.

For example, if Selector component 10852 is deemed unable to find an appropriate model using History Model Selector component 10858 or Research Based Builder component 10861, then Criterion component 10874 may configure Model Processor component 10863 to focus on Model Designer 10866 only, and stop the other attempts.

For real-time processing, Criterion component 10874 may also flag versions of models from the modules of Selector component 10852 that achieved reasonable results in the past, so that they may be used as intermediate solutions until new updates are available.

Orchestrator Perspective. From a more abstract, higher level point of view, system 10800 may be seen as an orchestrator-centered system 11200 managing all possible types of models, which may be organized in a graph, and which can be used for selecting processing paths, as illustrated in FIGS. 112a-c. Orchestrator 11200 may use any approach from logic and planning, supervised to unsupervised learning, reinforcement learning, search algorithms, or any combination of those.

Orchestrator 11200 may be viewed as a meta-component that combines input data types, processing history and objective, research knowledge, and situational context to determine the most appropriate Artificial Intelligence (AI) model for a given problem formulation, and may decide how the system should manage the processing resources, be it models or computing infrastructure.

Orchestrator 11200 may include components such as Model Selectors, such as Selector component 10852, Problem Qualifiers, such as Qualifier Component 10840, Planners, such as Planner component 10846, and Parallel Executors, such as Parallel Executor 10848.

Selector Component 10848 may generate, select, and prepare the appropriate models corresponding to each section of the processing plan, by searching 10858 for models in History Storage Component 10824 and searching 10861 for models in Research Knowledge in World Knowledge Component 10830, building new models from scratch 10866 based on determined type and morphology, and forming model ensembles 10870. It is to be noted that any type of machine learning model may be utilized by Selector Component 10848 for selection of models, as well as generation of models. For example, as shown in FIG. 112a, embodiments may utilize Supervised learning models 11202, such as Support Vector Machines models (SVMs) 11203, kernel trick models 11204, linear regression models (not shown), logistic regression models 11205, Bayesian learning models 11211, such as sparse Bayes models 11212, naive Bayes models 11213, and expectation maximization models 11214, linear discriminant analysis models(not shown), decision tree models 11215, such as bootstrap aggregation models 11216, random forest models 11217, and extreme random forest models 11218, deep learning models 11219, such as random, recurrent, and recursive neural network models (RNNs) 11220, long-short term memory models 11221, Elman models 11222, generative adversarial network models (GANs) 11224, and simulated, static, and spiking neural network models (SNNs) 11223, and convolutional neural network models (CNNs), such as patch-wise models 11226, semantic-wise models 11227, and cascade models 11228.

For example, as shown in FIG. 112c, embodiments may utilize Unsupervised learning models 11230, such as Clustering models 11236, such as hierarchical clustering models (not shown), k-means models 11237, single linkage models 11238, k nearest neighbor models 11239, k-medioid models 11240 mixture models (not shown), DBSCAN models (not shown), OPTICS algorithm models (not shown), etc., feature selection models 11231, such as information gain models 11232, correlation selection models 11233, sequential selection models 11234, and randomized optimization models 11235, feature reduction models, such as principal component analysis models 11242 and linear discriminative analysis models 11243, autoencoder models 11244, sparse coding models 11245, independent component analysis models 11246, feature extraction models 11247, Anomaly detection models (not shown), such as Local Outlier Factor models (not shown), etc., Deep Belief Nets models (not shown), Hebbian Learning models (not shown), Self-organizing map models (not shown), etc., Method of moments models (not shown), Blind signal separation techniques models (not shown), Non-negative matrix factorization models (not shown), etc.,

For example, as shown in FIG. 112b, embodiments may utilize Reinforcement learning models 11250, such as TD-lambda models 11251, Q-learning models 11252, dynamic programming models 11253, Markov decision process (MDP) models 11254, partially observable Markov decision process (POMDP) models 11255, etc. Embodiments may utilize search models 11260, such as genetic algorithm models 11261, hill climbing models 11262, simulated annealing models 11263, Markov chain Monte Carlo (MCMC) models 11264, etc. Likewise, Model Ensembler component 10870 may determine whether there is a combination of models that can outperform the selected model using any type of machine learning model.

Embodiments may have different specialized Domain Specific Instances of Selector Component 10848, each one optimized for a specific domain knowledge or problem context. Such instances may be deployed only in well delimited knowledge areas to achieve optimal efficiency and speed in problem solving tasks.

An example of general approaches 11300 (and a specific example from each one of them) that can be combined in the processing workflow of Selector Component 10848 is shown in FIG. 113. Approaches 11300 may include reasoning/logical planning 11302, connectionist/deep learning 11304, probabilistic/Bayesian networks 11306, evolutionary/genetic algorithms 11308, and reward driven/partially observable Markov decision process (POMDP) 11310.

Genetic Algorithms 11308 have been applied recently to the field of architecture search, mainly in the case of deep learning models. Due to improvements in hardware and tweaks in the algorithm implementation, these methods may show good results.

An exemplary, simple, intuitive, one-dimensional representation of this family of algorithms is shown in FIG. 114. In this example, elevation corresponds to the objective function and the aim is to find the global maximum of the objective function. An example of a genetic algorithm applied to digit strings is shown in FIG. 115. As shown in this example, starting with an initial population 11502, a fitness function 11504 may be applied and a resulting population may be selected 11506. Resulting populations may be comingled using crossover 11508 and mutations 11510 may be applied.

A high level pseudocode example reflecting this approach is given below.

```
START
Generate the initial population
Compute fitness
REPEAT
  Selection
  Crossover
  Mutation
  Compute fitness
UNTIL population has converged
STOP
```

Another example of a similar genetic algorithm 11600 is shown in FIG. 116. The approach includes an iterative process 11700, shown in FIG. 117. Process 11700 begins with 11702, in which new modeling architectures may be obtained and/or generated based on selection, crossover, and mutation. At 11704, the obtained configurations may be trained. At 11706, the surviving configurations may be selected based on how well they perform on a validation set. At 11708, the best architectures at every iteration will mutate to generate new architectures.

There are multiple options in terms of how the genetic algorithm may be implemented. For a deep neural net, an embodiment of a possible approach 11710 is shown in FIG. 117. The goal is to obtain an evolved population of models, each of which is a trained network architecture. At 11710 of process 11700, at each evolutionary step, two models may be chosen at random from the population. At 11712, the fitness of the two models may be compared and the worse model may be removed from population. At 11716, the better model may be chosen to be a parent for another model, through a chosen mechanism, such as mutation, and the child model may be trained. At 11718, the child model may be evaluated on a validation data set. At 11720, the child model may be put back in the population and may be free to give birth to other models in following iterations.

A large set of features may be optimized using genetic algorithms. Although originally genetic algorithms were used to evolve only the weights of a fixed architecture, since then genetic algorithms have been extended also to add connections between existing nodes, insert new nodes, recombine models, insert, or remove whole node layers, and may be used in conjunction with other approaches, such as back-propagation.

Support Vector Machines. In embodiments, Selector Component 10848 may train machine learning models for classifying the types of problems in a hierarchical structure. With this approach, the low-level features of the model may be processed and further used for detecting higher level characteristics (in a similar manner to the inner workings of a neural network). The data needed for the training of such models can be created from the corpus of existing research materials and results stored, for example, in History Storage Component 10824 and/or World Knowledge Component 10830. Machine learning may also be used for automating the task of creating a dataset.

In embodiments, Selector Component 10848 may use Support Vector Machine (SVM) processing, which, at its core, represents a quadratic programming problem that uses a separated subset of the training data as support vectors for the actual training.

A support vector machine may construct a hyperplane or set of hyperplanes in a high or infinite dimensional space, which may be used for classification, regression, or other types of tasks. Intuitively, a good separation may be achieved by the hyperplane that has the largest distance to the nearest training data points of any class (so-called functional margin), since in general the larger the margin the lower the generalization error of the classifier.

SVM solves the following problem:

$${min}_{w , b , ζ} \frac{1}{2} {w}^{T} w + C {\sum }_{i = 1}^{n} {ζ}_{i}$$

$$subject to {y}_{i} ( {w}^{T} ϕ ( {x}_{i} ) + b ) \geq 1 - {ζ}_{i} , \\ {ζ}_{i} \geq 0 , i = 1 , ... , n$$

for binary training vectors xᵢ∈ [symbol] ^{p}, and a vector y∈{1,−1}ⁿ.

The SVM model may be effective in high dimensional spaces (which gives the possibility of representing the problem formalization in more complex manner), and with smaller data sets (this is important because the existing research corpus has its limits in terms of availability and size). Different approaches may be chosen for multi-class problem classifications (“one against one”, “one vs the rest”), and different kernels may also be selected (linear, polynomial, rbf, sigmoid). In embodiments, a set of SVM models may be trained on a dataset that has as its features the problem characteristics and as its labels the solution module's characteristics. This may be done in a hierarchical way, so that different features of the solution may be predicted (model type, model morphology, model parameters, etc.).

The SVM model may take as an input the enhanced dataset and the qualifications for the problem formalization, both of which were constructed in Qualifier (Critic) Component 10840 using the History Storage Component 10824 and/or World Knowledge Component 10830 as primary sources of information.

Bayesian Networks. Embodiments may frame the problem of finding a suitable model for a problem in terms of an agent which tries to find the best action using a belief state in a given environment. Exemplary pseudocode for this formulation is presented below:
function DT-AGENT(percept) returns an action
   persistent: belief_state, probabilistic beliefs about the current state of the world action, the agent's action
   update belief_state based on action and percept
   calculate outcome probabilities for actions,
      given action descriptions and current belief_state
   select action with highest expected utility
      given probabilities of outcomes and utility information
   return action

This brings us to a new perspective, which directly highlights the uncertainty present in the task at hand, through the belief_state. Building on the known Bayesian Rule:

$$P ( cause \vert effect ) = \frac{P ( effect \vert cause ) P ( cause )}{P ( effect )}$$

we can use probabilistic networks for creating a module that is able to handle the uncertainty in the task in a more controlled manner.

A Bayesian network is a statistical model that represents a set of variables and their conditional dependencies. In embodiments, a Bayesian network may represent the probabilistic relationships between input data, situational context, and processing objective, and model types and morphologies. The network may be used to compute the probabilities of a model configuration being a good fit for a given problem formulation.

For example, given a problem formulation with two parameters A and B, we can use Bayesian networks to compute what is the probability that model M is a good candidate, given A and B. This may be formulated as shown at 11802 in FIG. 118.

For the simple independent causes network above we can write: p(M,A,B)=p(M A,B) p(A) p(B). It can be seen in the relationship above, features A and B are independent causes, but become dependent once M is known.

Embodiments may utilize various configurations that can be used for creating the Bayesian belief networks to determine the most appropriate model given the problem formulation features. For example, a converging belief network connection 11804 is shown in FIG. 118. The problem can also be defined as a chain of Mf related variables representing different features of the needed model, each corresponding to a single cause representing different features of the problem formulation, as shown at 11806 in FIG. 118. Network 11806 uses parallel causal independence. In this way, the final state of the model M is dependent on its previous values.

Embodiments may construct Bayesian Networks using a process 11900, shown in FIG. 119. A mathematical representation is shown below:

$$P ( {x}_{1} , ... , {x}_{n} ) = P ( {x}_{n} \vert {x}_{n - 1} , ... , {x}_{1} ) P ( {x}_{n - 1} , ... , {x}_{1 \\} )$$

$$P {x}_{1} , ... , {x}_{n} ) = {\prod }_{i = 1}^{n} P ( {x}_{i} \vert parents ( {X}_{i} ) )$$

$$P ( {x}_{1} , ... , {x}_{n} ) = P ( {x}_{n} \vert {x}_{n - 1} , ... , {x}_{1} ) P ( {x}_{n - 1} \vert {x}_{n - 2} , ... , {x}_{1} ) ... P ( {x}_{2} \vert {x}_{1} ) P ( {x}_{1} ) \\ = \\ {\prod }_{i = 1}^{n} P ( {x}_{i} \vert {x}_{i - 1} , ... , {x}_{1} ) . \\ P ( {X}_{i} \vert {X}_{i - 1} , ... , {X}_{1} ) = P ( {X}_{i} \vert Parents ( {X}_{i} ) )$$

Process 11900 may determine the set of variables that are required to model the domain. At 11902, the variables {X₁, . . . , Xₙ} may be ordered such that causes precede effects, for example, according to P(x₁, . . . , xₙ)=P(xₙ|xₙ₋₁, xᵢ)P(xₙ₋₁, . . . x₁). At 11904, for i=1 to n, 11906 to 11910 may be performed. At 11906, a minimal set of parents for Xᵢ may be chosen, such that P(Xᵢ|Xᵢ₋₁, . . . , X₁)=P(Xᵢ|Parents (X₁)). At 11908, for each parent, a link may be inserted from the parent to xᵢ. At 11910, a conditional probability table, P(Xᵢ|Parents (X₁)) may be generated.

In order to answer queries on the network, for example, embodiments may use a version of the Enumeration-Ask process 12000, shown in FIG. 120. Likewise, for inference on the network, embodiments may use a different version 12100, shown in FIG. 121.

Exact inference complexity may depend on the type of network, accordingly, embodiments may use approximate inference to reduce complexity. For example, approximate inference processes such as Direct Sampling, Rejection Sampling, and Likelihood Weighting may be used. An example of a Likelihood Weighting process 12200 is shown in FIG. 122.

Instead of generating each sample from scratch, embodiments may use Monte Carlo Markov Chain algorithms, to generate each sample by making a random change to the preceding one. For example, Gibbs Sampling 12300, shown in FIG. 123, is such a starting point approach. A mathematical representation 12302 of Gibbs sampling is also shown.

Embodiments may estimate any desired expectation by ergodic averages—computing any statistic of a posterior distribution using N simulated samples from that distribution:

$${E [ f ( s ) ]}_{𝒫} \approx \frac{1}{N} {\sum }_{i = 1}^{N} f ( {s}^{( i )} )$$

where [symbol] is the posterior distribution of interest, f(s) is the desired expectation, and f(s⁽ⁱ⁾) is the ith simulated sample from [symbol] .

Model Combination. For any given situation, Selector 10852 may not be constrained to using a single model, but may activate a combination of models for ensemble learning, for example, to minimize bias and variance. Embodiments may use various tools to determine models to combine. For example, embodiments may use cosine similarity, in which the results from different models are represented on a normalized vector space. The general formula for cosine similarity is:

$$\vec{a} \cdot \vec{b} = ‖ \vec{a} ‖ ‖ \vec{b} ‖ cos θ$$

$$cos θ = \frac{\vec{a} \cdot \vec{b}}{‖ \vec{a} ‖ ‖ \vec{b} ‖}$$

Accordingly, cos θ may be used as a metric of congruence between different models. However, embodiments may also use less correlated models, which learn different things, to broaden the applicability of the solution.

Application Areas. Embodiments may provide improved flexibility and scalability. For example, embodiments may be adapted for a large array of existing problems, and also extended for new approaches. For example, possible application areas may include, but are not limited to:

Anthropomorphism in Human-Machine Interaction. Personality emulation. There are two facets of anthropomorphism. On the one hand, we can call a system anthropomorphic when it can imitate human characteristics. Due to this capability, embodiments may emulate human personality, according to user preferences, and have, for example, a sarcastic mood or maybe have a very cheerful disposition.

Embodiments may achieve this by having models trained on different datasets to obtain different personality traits in how the system interacts with users. Embodiments may use a critic 10840-selector 10852 paradigm that will select the best model to be used based on the explicit preference of the user or the inferred most appropriate choice. An example of a critic 10840-selector 10852 mechanism on a personality layer is shown in FIG. 124.

Emotional intelligence. Embodiments may be anthropomorphic when it adapts to a human's profile. For example, if embodiments act as a learning assistant, they may tailor the content and review methods in a way that best matches the user's learning abilities. For example, when embodiments act as an activity recommender engine, they may adapt recommendations to the user's skills, pace, and time. Embodiments may provide this second type of anthropomorphism by being perceptive about the user's disposition or feelings and adjusting the frequency and type of interaction that is initiated.

Brain Disease Diagnostics and Treatment and Medical Devices for Cognitive Enhancement. Neural modulation solutions for the treatment of neurodegenerative diseases (NDD) may involve the recording of large amounts of data to enable using techniques of machine learning for diagnosing and monitoring of the condition of the brain. Besides their benefit in NDD therapy, neuromodulation techniques may be used for the enhancement of different cognitive functions, such as memory, language, concentration, etc. These tasks may require the processing of large amounts of data employing a variety of AI models. Embodiments may handle these kinds of scenarios as well.

Intention Awareness Manifestation (IAM). Embodiments may provide an intelligent system for the definition, inference, and extraction of the user's intent and aims using a comprehensive reasoning framework for determining user intents.

User intent identification becomes significantly important with the increase in technology, the expansion of digital economies and products and diversity in user preferences, which positions a user as a key actor in a system of decisions. Interpretation of such decisions or intent inference may lead to a more open, organized, and optimized society where products and services may be easily adapted and offered based on a forecast of user intent and preferences, such as provided by a recommendation system. Crime and social decay may be prevented using data and intent analysis, such as provided by a prevention system, and the common good may be pursued by optimizing every valuable aspect of user's dynamic lifestyle, such as provided by a lifestyle optimization system. Embodiments may provide these features both at the level of the community and of the individual.

Embodiments of the present systems and methods may be well suited to providing IAM functionality due to the large diversity of data channels and types together with the high complexity and interrelatedness of different ontologies that are involved.

Quantified Self Quantified self, also known as lifelogging, is a function that tries to incorporate technology into data acquisition on aspects of a person's daily life. People may collect data in terms of electroencephalogram (EEG), electrocardiogram (ECG), breathing monitoring, food consumed, quality of surrounding air, mood, skin conductance, pulse oximetry for blood oxygen level, and performance, whether mental or physical.

The logging of all these parameters results in a large amount of recorded data from which one could really benefit if one can extract meaning through processing the data. Given the diversity of the sensors used and the resulting diversity of the recorded data types, the machine learning models employed for data processing need to be carefully chosen and tuned to enable meaningful results. Embodiments of the present systems and methods may provide a powerful platform that can absorb the input data and automatically find or create the most appropriate model for the given dataset.

The field of quantified self may bring important benefits not only due to the ability of monitoring different aspects of our being but also to the possibility of early disease detection that increases as research in the life sciences progresses.

Automated Manufacturing Systems. Automation in manufacturing can transform the nature of manufacturing employment, and the economics of many manufacturing sectors. Embodiments of the present systems and methods may contribute to the new automation era: rapid advances in robotics, artificial intelligence, and machine learning to enabling machines to match or outperform humans in a range of work activities, including ones requiring cognitive capabilities. Industries can use automation provided by embodiments to address a number of opportunities, including increasing throughput and productivity, eliminating variation, and improving quality, improving agility, and ensuring flexibility, and improving safety and ergonomics.

Energy Management. By implementing autonomous reasoning in energy systems, improvements can be achieved to the efficiency, flexibility, and reliability of a site energy by analyzing, monitoring, and managing a site and associate optimization priorities over time. Embodiments may provide a customer-centric energy system providing improved energy efficiency, cost minimization and reduced CO₂ emissions.

Transportation. Embodiments may provide features for automated and connected vehicle technologies and for the development of autonomous cars, connected cars, and advanced driver assistance systems. Embodiments may be applied to autonomous connected vehicles, where vehicles that use multiple communication technologies to communicate with the driver, such as to other cars on the road (vehicle-to-vehicle [V2V]), roadside infrastructure (vehicle-to-infrastructure [V2I]), and the “Cloud” [V2C]. Embodiments may be used to not only improve vehicle safety, but also to improve vehicle efficiency and commute times and facilitate autonomy in use.

Infrastructure. Data Service. A data Processing Service may be responsible for collecting data from different input channels 10802, decompressing the data, if necessary, and storing it for later use.

There may be a large number of data channels 10802 that send data to system 10800. Embodiments may store such data on the Cloud, providing a need for high scalability in recording this data, as well the capability to store a large amount of data.

There are different technologies which can support this. For example, embodiments may use those that provide the constant increase of inputs and high parallelism of incoming data and may be based on the Publish/Subscribe Paradigm. In this specific case of data processing, the inputs may act as data publishers while the system 10800, which processes the data, may act as a sub scriber.

An exemplary embodiment 12500 of architecture and the components that may provide data ingestion and data processing is shown in FIG. 125. This architecture and the components are merely examples. Embodiments may utilize other architectures and components as well.

As shown in the example of FIG. 125, embodiments may include, stream-processing software 12502, such as Apache Kafka, for data streaming and ingestion. Stream-processing software 12502 may provide real-time data pipelines and streaming apps, and may be horizontally scalable, fault-tolerant, and very fast.

Data coming from different input channels 12504 may be distributed for processing over, for example, the Internet 12506, to Data Processing Service 12508, which may be implemented in the Cloud. Embodiments may deploy Data Processing Service 12508 in one or more nodes.

Embodiments may be implemented using, for example, Apache Kafka Security with its versions TLS, Kerberos, and SASL, which may help in implementing a highly secure data transfer and consumption mechanism.

Embodiments may be implemented using, for example, Apache Kafka Streams, which may ease the integration of proxies and Data Processing Service 12508.

Embodiments may be implemented using, for example, Apache Beam, which may unify the access for both streaming data and batch processed data. It may be used by the real time data integrators to visualize and process the real time data content.

Embodiments may utilize a high volume of data and may have large data upload and retrieval performance requirements. Embodiments may use a variety of database technologies, such as OpenTSDB (“OpenTSDB—A Distributed, Scalable Monitoring System”), Timescale (“OpenTSDB—A Distributed, Scalable Monitoring System”, “Timescale an Open-Source Time-Series SQL Database Optimized for Fast Ingest, Complex Queries and Scale”), BigQuery (“BigQuery—Analytics Data Warehouse Google Cloud”), HBase (“Apache HBase—Apache HBase™ Home”), HDF5 (“HDF5®—The HDF Group”), etc.

Embodiments may be implemented using, for example, Elasticsearch, which may be used as a second index to retrieve data based on different filtering options. Embodiments may be implemented using, for example, Geppetto UI widgets, which may be used for visualizing resources as neuronal activities. Embodiments may be implemented using, for example, Kibana, which is a charting library that may be used on top of Elasticsearch for drawing all types of graphics: bar charts, pie charts, time series charts etc.

Implementation Languages. Embodiments may be implemented using a variety of computer languages, examples of which are shown in FIG. 108. For example, Problem Formalization component 10816 may be implemented using Scala, Haskell, and/or Clojure, Qualifier (Critic) component 10846 may be implemented using Julia and/or C++, Planner component 10846 may be implemented using C++ and/or Domain Specific Languages, Selector component 10852 may be implemented using Python and C++, Parallel Executor component 10848 may be implemented using Erland and/or C++, Module Scheduler component 10854 may be implemented using C++, Solution Processor component 10856 may be implemented using C++

World Knowledge: may be implemented using Scala, Haskell, and/or Clojure, History Knowledge component 10824 may be implemented using Scala, Haskell, and/or Clojure, Infrastructor component 10875 may be implemented using C++

Implementation Details. Embodiments may be deployed, for example, on three layers of computing infrastructure: 1) a sensors layer equipped with minimal computing capability may be utilized to accommodate simple tasks (such as average, minimum, maximum), 2) a gateway layer equipped with medium processing capability and memory may be utilized to deploy a pre-trained neural network (approximated values), and 3) a cloud layer possessing substantial processing capability and storage may be utilized to train the models and execute complex tasks (simulations, virtual reality etc.).

Embodiments may employ a diverse range of approximation methods, such as Parameter Value Skipping, Loop Reduction and Memory Access Skipping or others greatly facilitation reduction in complexity and adaptation for non-cloud deployment, such as the gateway layer. The entire processing plan may also utilize techniques from Software Defined Network Processing, Edge Computing Techniques, such as Network Data Analysis and History Based Processing Behaviors Learning using Smart Routers.

In embodiments, the three layer computing infrastructure (cloud, gateway, sensors) may provide flexibility and adaptability for the entire workflow. To provide the required coordination and storage, cloud computing may be used. Cloud Computing is a solution which has been validated by a community of practice as a reliable technology for dealing with complexity in workflow.

In addition to the cloud layer, embodiments may utilize Fog/Edge Computing techniques for the gateway layer and sensors layer to perform physical input (sensors) and output (displays, actuators, and controllers). Embodiments may create small cloud applications, Cloudlets, closer to the data capture points, or nearer to the data source and may be compared with centralized Clouds for determining benefits in terms of costs and quality-of-results. By nature, these cloudlets may be nearer to the data sources and thus minimize network cost.

This method will also enable the resources to be used more judiciously, as idling computing power (CPUs, GPUs, etc.) and storage can be recruited and monetized. These methods have been validated in Volunteer Computing which has been used primarily in academic institutions and in community of volunteers (such as BOINC).

For example, in embodiments, Solution Processor component 10856, which runs the solution modules, may be mapped to 3 different layers: (i) sensors layer (edge computing), (ii) gateway layers (in-network processing) and (iii) cloud layer (cloud processing). Starting with sensors layer, the following two layers (gateway layers and cloud layers) may add more processing power but also delay to the entire workflow, therefore depending on task objectives, different steps of the solution plan can be mapped to run on different layers.

Edge Computing implies banks of low power I/O sensors and minimal computing power; In-Network Processing can be pursued via different gateway devices (Phones, Laptops, and GPU Routers) which offer medium processing and memory capabilities; Cloud Computing may provide substantial computation and storage.

In embodiments, the learning modules may be optimized for the available computing resources. If computing clusters are used, models may be optimized for speed, otherwise, a compromise between achieving an higher accuracy and computing time may be made.

An exemplary block diagram of a computer system 12600, in which processes involved in the embodiments described herein may be implemented, is shown in FIG. 126. Computer system 12600 may be implemented using one or more programmed general-purpose computer systems, such as embedded processors, systems on a chip, personal computers, workstations, server systems, and minicomputers or mainframe computers, or in distributed, networked computing environments. Computer system 12600 may include one or more processors (CPUs) 12602A-12602N, input/output circuitry 12604, network adapter 12606, and memory 12608. CPUs 12602A-12602N execute program instructions in order to carry out the functions of the present communications systems and methods. Typically, CPUs 12602A-12602N are one or more microprocessors, such as an INTEL CORE® processor. FIG. 126 illustrates an embodiment in which computer system 12600 is implemented as a single multi-processor computer system, in which multiple processors 12602A-12602N share system resources, such as memory 12608, input/output circuitry 12604, and network adapter 12606. However, the present communications systems and methods also include embodiments in which computer system 12600 is implemented as a plurality of networked computer systems, which may be single-processor computer systems, multi-processor computer systems, or a mix thereof.

Input/output circuitry 12604 provides the capability to input data to, or output data from, computer system 12600. For example, input/output circuitry may include input devices, such as keyboards, mice, touchpads, trackballs, scanners, analog to digital converters, etc., output devices, such as video adapters, monitors, printers, etc., and input/output devices, such as, modems, etc. Network adapter 12606 interfaces device 12600 with a network 12610. Network 12610 may be any public or proprietary LAN or WAN, including, but not limited to the Internet.

Memory 12608 stores program instructions that are executed by, and data that are used and processed by, CPU 12602 to perform the functions of computer system 12600. Memory 12608 may include, for example, electronic memory devices, such as random-access memory (RAM), read-only memory (ROM), programmable read-only memory (PROM), electrically erasable programmable read-only memory (EEPROM), flash memory, etc., and electro-mechanical memory, such as magnetic disk drives, tape drives, optical disk drives, etc., which may use an integrated drive electronics (IDE) interface, or a variation or enhancement thereof, such as enhanced IDE (EIDE) or ultra-direct memory access (UDMA), or a small computer system interface (SCSI) based interface, or a variation or enhancement thereof, such as fast-SCSI, wide-SCSI, fast and wide-SCSI, etc., or Serial Advanced Technology Attachment (SATA), or a variation or enhancement thereof, or a fiber channel-arbitrated loop (FC-AL) interface.

The contents of memory 12608 may vary depending upon the function that computer system 12600 is programmed to perform. In the example shown in FIG. 126, exemplary memory contents are shown representing routines and data for embodiments of the processes described above. However, one of skill in the art would recognize that these routines, along with the memory contents related to those routines, may not be included on one system or device, but rather may be distributed among a plurality of systems or devices, based on well-known engineering considerations. The present communications systems and methods may include any and all such arrangements.

In the example shown in FIG. 126, memory 12608 may include Data Sources routines 12610, API 12612, Problem Formalization routines 12614, History Storage routines 12616, World Knowledge routines 12618, Qualifier (Critic) routines 12620, Planner routines 12622, Parallel Executor routines 12624, Module Scheduler routines 12626, Selector routines 12628, Solution Processor routines 12630, Infrastructor routines 12632, and operating system 12634. Data Sources routines 12610 may include software to perform the functions of Data Sources component 10802, as described above. API 12612 may include software to perform the functions of API 10814, as described above. Problem Formalization routines 12614 may include software to perform the functions of Problem Formalization component 10816, as described above. History Storage routines 12616 may include software to perform the functions of History Storage component 10824, as described above. World Knowledge routines 12618 may include software to perform the functions of World Knowledge component 10830, as described above. Qualifier (Critic) routines 12620 may include software to perform the functions of Qualifier (Critic) component 10840, as described above. Planner routines 12622 may include software to perform the functions of Planner component 10846, as described above. Parallel Executor routines 12624 may include software to perform the functions of Parallel Executor component 10848, as described above. Module Scheduler routines 12626 may include software to perform the functions of Module Scheduler component 10854, as described above. Selector routines 12628 may include software to perform the functions of Selector component 10852, as described above. Solution Processor routines 12630 may include software to perform the functions of Solution Processor component 10856, as described above. Infrastructor routines 12632 may include software to perform the functions of Infrastructor component 10875, as described above. Other operating system routines 12622 may provide additional system functionality.

Fundamental Code Unit

FIG. 127 is a high-level representation of FCU/MCP device determining specific FCU signal patterns and producing signals affecting cell functioning by invasive and non-invasive stimulation. This illustration is primarily concerned with the relationship between the read modality and write modality.

FIG. 128 is a high-level representation of coprocessor functions for implementing the manipulation of cellular structures via signaling, as outlined in FIG. 127. Signaling includes the controlled release of S (+) and R (−) isomer/enantiomer combinations to specific brain regions and neural networks. FIG. 128 serves to distinguish the chemical action of the FCU/MCP versus the blanket pharmaceutical interventions currently available.

FIG. 129 is an example of an apparatus implementing the invention, demonstrating the interconnections and functions of its composite parts. This includes both the read (input) and write (output) components of FCU/MCP.

FIG. 130 is a hardware implementation of the read and write modality hierarchy that illustrates the interaction between the coprocessor itself and its many sources of input. FIG. 130 also includes the database of existing patterns, querying routines, pattern analysis routines, and finally, input from the physiological system being analyzed.

FIG. 131 is an illustration of the read/write modality usage in the detection and treatment of a neurological disorder, Alzheimer's disease. The read modality, multimodal body sensor networks (mBSN), gathers data from presumptive Alzheimer's patient: movement/gait information from arms and legs and cognitive information from audio speech sensors. This information is sent to the Analyzer, through Interface 1, a Sensor control. The Analyzer computes unary mathematics (+/−) of the incoming motion and speech information and also computes unary delivery (S+/R−) of write modalities for treatment of Alzheimer's disease. Through Interface 2, the Analyzer configures an ultrasound Effector, which creates an ultrasonic beam that temporarily permits a narrow opening of the blood brain barrier to enable delivery of enantioselective acetylcholine esterase inhibitors (AChE). AChE is delivered directly to the hippocampus to treat Alzheimer's disease.

FIG. 132 provides a higher-level view of the relationship between sensors, or read modality elements, and effectors, or write modality elements. Each of these exists in a cyclic relationship with the next. The dual process of querying by read modalities and application of write modalities varied by type, duration, and intensity is computed by unary mathematics of FCU and is used to diagnose and treat complex neurological disorders.

FIG. 133 illustrates the translation of neural code, from neurotransmitter and spike/pulse sequences, to action potentials, to frequency oscillations, and finally to cognitive output including speech and behavior. Original neural encoded information might be meaningful however, the meaning is not dependent on the interpretation. In neurological disorders, post-synaptic neurons may not be able to interpret and act on meaningful encoded messages that are transmitted to it.

FIG. 134 is a detailed schematic of the multiple levels at which the FCU analyzer operates, ranging from the subatomic (charged particle) level to the molecular neurotransmitter and finally the linguistic level.

FIG. 135 is a flow diagram of the process of autofluorescence.

FIG. 136 is a flow diagram of a proposed FCU-based mechanism for exchanging information within the brain: endogenous photon-triggered neuropsin transduction.

FIG. 137 is an example of an apparatus implementing the invention, demonstrating the interconnections and functions of its composite parts. This includes both the read (input) and write (output) components of FCU/MCP.

FIG. 138 illustrates photonic transduction in NAH Oxidase (NOX) and NAD(P)H. Both of these molecules are affected by light, and the emission of near-UV electromagnetic energy by NOX causes a similar reaction in neuropsin, whose emitted light wavelength can be used to interpret brain activity. What results is a neuropsin-regulated signaling transduction cascade, since the photon energy emitted by NOX is higher than the threshold required to change neuropsin's conformality.

Embodiments may include a sensor for the detection of dopamine levels inside the neurocranium. The sensor may be fabricated on a silicon substrate using vertically aligned carbon nanotubes as sensitive electrodes which may be connected to a signal generator and a wireless platform in order to allow remote analysis, similar to the KIWI device described herein. The carbon nanotubes may be specifically functionalized to increase the detection sensitivity of the sensor and decrease false-positive read-outs. The dopamine sensor may be integrated on a proprietary wireless platform. The dopamine detection sensor integrated with the wireless data acquisition platform proposed may be tested in vitro on controlled solutions in order to validate it.

Neurotransmitters (NTs) are chemical messengers between neurons and other cells having low extracellular concentrations. They are difficult to detect especially in the presence of other electro active chemicals present in the brain. Generally, the human neurotransmitters belong to amino acids class such as glutamic acid, to biogenic amines group such as epinephrine and dopamine and to soluble gases group such as nitric oxide. NTs play an important role in the brain functions, such as behavior and cognition, and the changes in their concentration in the central nervous system have been correlated with schizophrenia, dementia, and other neurodegenerative diseases associate with elder age. Autism and physical illnesses such as glaucoma, shortage of thyroid hormone are related to neurotransmitters level as well. In fact, the cardiovascular and renal functions systems involved in establishing the integration brain-body are affected and controlled in their behavior by concentration of such messengers influencing sleeping, mood, memory, and appetite. In our time with a significant increase of life span, neurodegenerative diseases became more important to be treated and neurotransmitters need more detection and control. One of the well-known neurotransmitters is Dopamine (3,4-dihydroxyphenethylamine, DA), which modulates several aspects of brain circuits. Functions of dopamine are related to movement, to memory, to attention, to pleasure and understanding rewards, to mood and processing pain, to behavior and cognition, to sleep, to creativity and personality. For neurochemical studies, dopamine is the major test compound studied. Dopamine is a cation and at physiological value of pH has basal extracellular levels around 0.01-0.03 μM. At such value of pH, the dopamine detection limit is strongly dependent on sensor and on determination method. The development of selective measurement of dopamine at the low levels characteristic of living system (26-40 nmol L−1 and below) can make a great contribution to disease diagnosis. Due to the electroactive nature of dopamine, prior efforts have been made into various approaches to introduce sensitive and inexpensive devices for rapid detection up to now, but challenges are still present, limiting the promotion of known electrodes, in particular for in vivo applications due to their size with a more than 1 mm in diameter. Such dimension causes significant tissue damage. For voltammetry detection, most of traditional electrodes present low selectivity, with dopamine oxidation peak overlapping with common interferences such as uric and ascorbic acid whose concentrations are usually around 102-103 times higher in biological systems.

Dopamine belongs to a class of substances known as catecholamines, which are monoamine neurotransmitters. Other catecholamines may include epinephrine and norepinephrine.

Carbon based materials have been used largely in electrochemical sensors, due to their electron transfer kinetics and surface adsorption based on electrostatic interactions. In recent years, several research groups have employed carbon nanotubes (CNT) as electrodes for monitoring biological structures by specific functionalization of the surface in order to render them biocompatible for use in vitro, as well as in vivo. By integrating CNTs in electrochemical sensors, it may be possible to significantly improve their performance due to higher electron transfer kinetics and lower detection limits, compared to classical carbon-based electrodes. CNT based sensors can be used in different electrochemical characterization methods like voltammetry, amperometry, potentiometry, and electrochemical impedance spectroscopy. Carbon nanotubes have the advantage of easily binding to biological materials and to enter body cells by endocytosis. Of these, single walled carbon nanotubes (SWCNTs) have special characteristics. Because they create very stable suspensions in physiological buffers and are suitable to be used in biological environments. The bonds with the attached molecules are easily destroyed by certain enzymes. The nanostructured surface provides a larger specific surface area, increased interfacial adsorption, and enhanced electrocatalytic activity. Thus, CNT-based electrodes have rapid electron transfer, reduced electrode fouling, reduced overpotential, and increased sensitivity and selectivity for neurotransmitter detection. Carbon nanotubes (CNTs), graphene, and their derivatives have been used for neurotransmitter detection, either by themselves or in conjunction with polymers or metal nanoparticles.

It is worth mentioning that new types of carbon nanomaterials beyond CNTs, such as various forms of graphene, carbon nanohorns, graphene nanofoams, graphene nanorods, and graphene nanoflowers are now increasingly used for sensors. Frequently the sensing of biomolecules employs enzymes in detection, but due to their denaturation enzymeless or enzyme-containing carbon-nanomaterial-based biosensors are preferred. Due to agglomerations absence, directly growing vertically aligned CNT are leading to reproducible surfaces with CNT exposed ends. Such ends, having defect sites available to be functionalized with oxygen containing groups are able to adsorb cationic dopamine selectively repealing other anionic compounds at the same pH such as uric acid pH.

Several studies in the literature demonstrate the increase in selectivity of Nafion®-coated sensors in the determination of catecholamines in biological fluids minimizing the effect of some endogenous interferences. Nafion® consists of a tetrafluoroethylene main chain with perfluoroether side chains terminated with a sulfonic acid group. The Nafion®-induced solubilization of CNT permits a variety of manipulations, including modification of electrode surfaces and preparation of biosensors. The distinct advantages of the CNT/Nafion® coating were exploited also for dramatically improving the detection of catecholamine neurotransmitters in the presence of the common ascorbic acid interference. A strategy used for vertically-aligned CNTs was to grow them on a sensor surface using chemical vapor deposition (CVD). A solid phase buffer layer and catalyst deposited on the substrate was proposed by. Xiang et al. Their system for the detection of dopamine and ascorbate involved vertically-aligned, carbon nanotube sheathed carbon fibers and permitted good sensitivity for in vivo measurements.

Embodiments may include the fabrication of a custom dopamine sensor integrated with a wireless platform for data acquisition and signal injection for the development of a miniaturized implantable biochip.

In embodiments, the dopamine sensor may be based on specific requirements. As described herein, a wireless platform, able to record up to 256 channels with 16 bit resolution at a 30 kS/s may be used. The hardware platform is able to interface both with commercially available probes and with custom probes and headstage. The prototype platform has been tested end-to-end with commercially available sensing structures; the test setup involved measuring injected signal through a PBS droplet. In embodiments, the platform may be interfaced with a custom-made dopamine sensor and validated for future developments of diagnosis and prevention tools for neurodegenerative disorders.

Embodiments may include a wireless sensor for the detection of dopamine concentrations. Embodiments may provide optimized growth of vertically aligned carbon nanotubes tailored for increased sensitivity; specific functionalization of carbon nanotubes for increased selectivity; an electrochemical sensing structure using functionalized carbon nanotubes as electrodes; integration of the sensing structure with the wireless platform; and validation of the sensor.

An exemplary system architecture of a dopamine sensor system 14000 is shown in FIG. 140. Such a system may provide a small, self-contained, wirelessly connected, wirelessly charged, AI-driven replacement for current deep brain stimulation (DBS) solutions. The device may be implanted in the neurocranium. The implant may record brain activity and deliver electrical and optical signals to target areas, with algorithms that will determine the therapeutic response and be continually improved by machine learning. It may be implanted using a minimally-invasive surgical procedure. A cloud-based software platform may then securely collect and interpret information in real-time. At the current development stage the system is able to record up to 256 channels with 16-bit resolution at a 30 kS/s. The hardware platform may be able to interface both with commercially available probes and with the custom probes and headstage currently under development.

In the example shown in FIG. 140, system 14000 may include an interface with a PC via wired and/or wireless connection, for future migration to battery powered platform; Interface with neural probes, such as Neuronexus, Cambridge Neurotech, Plexon; raw data recording in a MicroSD card; neural stimulation with micrometer precision on up to 16 sites/probe; online processing and spike detection. A suite of PC based software tools may provide the capability to analyze and plot the acquired signals by detecting the geometrical position of the neural activity sources.

Neural circuits and networks form the neurophysiological foundation for neural signal transformation in the nervous system of the brain. The basic neural network connector is the synapses among neurons, which behave as a diode as that in electronic circuits in order to facilitate unidirectional neural information transmission. A post synaptic coupling allows the third neuron to control the synaptic connection between a pair of neurons, which converts the synaptic diode to a transistor where the synaptic gate is controlled by the inhibitory or excitatory signal of the third neuron.

A fundamental observation in neuroanatomy and neurophysiology is that the neural signals transmitting in neural networks are unified. The uniform neural signals are spikes (electrochemical impulse) relayed through axons, synapses, and dendrites between neurons as shown in FIG. 141, which illustrates the generation and transmission of neural signals in the nervous system.

The neural spikes are uniform in neural networks among all outputs of sensory neurons, inputs of all motor neurons, and between both ends of association neurons. The coding mechanism of the uniformed neural signals is illustrated as shown in FIG. 142, which illustrates the waveform of neural spikes, according to neurological experiments. A normalized neural spike can be numerically simulated as shown in FIG. 143, which illustrates a simulation of the neural spike in MATLAB.

Definition 1. The uniformed neural signal, s(t), known as spikes transmitted in the nervous system can be formally modeled as an impulse function, i.e.:

$$s ( t ) = \frac{cos ( \frac{πt}{δ} )}{1 - ( \frac{4 {t}^{2}}{{δ}^{2}} )} \qquad ( 1 )$$

where t is the variable along time, δ the pace factor (8=2.5 ms typically) that controls the width of the spike w (w=4δ=10 ms), and the amplitude of the neural spike is normalized in the range.

According to Eq. 1, the shape of a neural spike, s(t), is plotted as shown in FIG. 143, where the width of the spike is enlarged for 40 times, i.e., w′=400 ms, for clarity of the details of the unique neural signals. The numerically generated impulse is quite closer to the observations in neurology as shown in FIG. 142 while it is much more rigorous for formal analyses.

Definition 2. The absolute amplitude of neural spikes, |s(t)|, transmitting in the nervous system is ranged in [−70 mV, 30 mV], i.e.:

−70 mV≤|s(t)|≤30 mV (3)

However, a more convenient representation of the relative amplitude of normalized neural spikes, s(t)∥, is in a positive range 0 mV≤|s(t)∥≤100 mV corresponding to a spike s(t)|s, i.e.:

$$s ( t ) \vert s \{ \begin{matrix}1 , θ \leq \vert s ( t ) \vert \vert \leq 100 mV \\ 0 , otherwise\end{matrix} \qquad ( 4 )$$

where θ is a given threshold which is typically θ=20 mV, and is the normalized unit type suffix.

The normalization of neural spikes is implemented by all sensory receptors where various forms of external stimuli are unified into electronic neural signals, and different ranges of external stimuli are normalized in the range of [−70 mV, 30 mV] where −50 mV is considered as the threshold of a neural spike's presence equivalent to θ=20 mV in the relative range [0 mV, 100 mV] of neural spikes. The threshold of neural spikes provides a 20 mV margin for noise tolerance in the nervous system. In addition, the relativity of signal strengths as represented by the change of number of spikes enables a robust mechanism for noise resistance and fault tolerance in nervous systems of the brain.

The Spike Frequency Modulation. In the preceding section, the formal model of neural signals is uniformed by neural spikes. The spikes are generated by sensory neurons and then transferred in the nervous system relaying by associated neurons. This section describes the generation of neural signals as sequences of spikes by a sensory neuron, which leads to spike frequency modulation. Corresponding to the relative range [0, 100 mV] of the normalized input stimuli, the rate of spikes generated by a sensory neuron is proportional to the strength of the analog input is 0-100 spikes per second (sps). It is up to a hundred times faster than that of the arterial rate, which is normally ranged within 66-75 pulses per minute (ppm) or 1.1-1.2 sps. In other words, the average period of neural spikes is 10 ms in the central and peripheral nervous systems.

In order to unify the neural signals within the nerves systems of the brain, any type of external stimuli is transformed into the form of normalized neural spikes as shown in FIG. 143 and Eq. 1 via specific sensory receptors. This transformation process is formally explained by the following.

Definition 3. Spike frequency modulation (SFM) is a signal transform function that converts an analog stimulus on a sensory neuron into a sequence of spikes where the rate of spikes per second (sps) is proportional to the intensity of the input in mV, i.e.:

$$SFM {f}_{S F M} : {s}_{i} ( t ) \vert mV \to {s}_{o} ( t ) \vert sps = \\ {s}_{o} ( t ) \vert sps = \\ \{ \begin{matrix}k \vert s i ( t ) \vert mV \vert , θ \leq s i ( t ) \vert mV \leq 100 mV \\ 0 , otherwise\end{matrix} \qquad ( 4 )$$

where k is the conversion factor, k=1 [sps/mV], θ is the sensory threshold whose typical value is 0=20 mV, and |sps or |mV is the type suffixes of SFM, respectively.

It is noteworthy that both the rate and amplitude of neural spikes, so (t) and | so (t) 1, are constrained within certain ranges, respectively. This is a natural mechanism of neurons for signal saturation in order to protect the nervous system from any potential overload.

Experiment 1. Let the input of a dynamic stimulus to a sensory neuron be a polynomial curve sᵢ=−0.3t⁴+3.1t³−10.2t²+12.9t−0.3 unified in the relative range [0, 100 mV] as shown in FIG. 144, which illustrates the results of an experiment on spike frequency modulation (SFM). The output sequence of neural spikes, so (t), is generated by SFM according to Eq. 4 in the range of [0, 100 sps], where θ determines the sensibility of the given neuron.

The output of the SFM neural signal is a sequence of spikes that is modulated by the rate of impulses proportional to the strengths of the input in each sample period τ. As a result, the SFM sequence of spikes in the given points are [0, 0, 20, 58] determined in the beginnings of the four sampling periods.

Theorem 1. The signaling principle of neurology states that the general form of neural signals in the brain and body is unified by the spike signals generated by SFM.

Proof. Because any neural signal is originated from a sensory nervous by SFM, and then transmitted in the nervous system, the generality of Theorem 1 is proven.

The SFM principle is supported by observations and experimental data in neuroanatomy and neurophysiology.

Corollary 1. The rate and amplitude of SFM signals, sₒ (t) and |sₒ (t)|, are unified, respectively, in the following ranges:

$$= \{ \begin{matrix}0 \leq {s}_{0} ( t ) \leq 100 sps \\ 0 \leq {s}_{i} ( t ) \vert V \vert {s}_{0} ( t ) \leq 100 mV\end{matrix} \qquad ( 5 )$$

Proof. Corollary 1 can be proved by Theorem 1 and Definition 3.

SFM may be used to explain a variety of phenomena of human cognition and behaviors in neuroinformatics, cognitive science, brain science, cognitive computing and medical science.

Spike frequency demodulation (dSFM). As neural signal modulation is embodied by the sensory neurons, demodulation is implemented by the motor neurons in human nervous systems where the former are input-oriented and the latter are output oriented. Demodulation of neural signals transforms the internal SFM sequences of spikes into analog effecters, typically as a step function, in order to drive muscles and gestures of the head and body.

The demodulation of neural signals is an inverse operation of SFM, which can be illustrated similar to that of FIG. 144 where the input and output are interchanged. Demodulation of the spike sequences into the analog counterpart is equivalent to a left rectangular numerical integration of the SFM signals for each sampling period determined by a given threshold of the conversion.

Definition 4. Demodulation of spike frequency modulated signals, dSFM, is an inverse SFM function that transforms a sequence of spikes into an analog signal whose amplitude is proportional to the rate of the input SFM signals, i.e.:

$$d S F M {f}_{dSFM} : {s}_{i} ( t ) \vert sps \to {s}_{0} ( t ) \vert mV \\ = {s}_{0} ( t ) \vert mV = {\int }_{0}^{t} {s}_{i} ( t ) \vert sps dt \\ = {k}^{′} {Σ}_{i = 0}^{\vert r \vert} {s}_{i} ( t ) \vert sps [ mV ] \qquad ( 6 )$$

where k′ is a conversion factor, k′=1.0 [mV/sps], and T is the sampling or transforming period typically τ=10 ms.

Experiment 2. Let the input be a sequence of spikes applied to a motor neuron corresponding to a polynomial s₀(t)=−0.3t⁴+6.1t³−3.2t²+6.9t−0.3 in [0, 10τ] unified in the relative range [0, 100 mV] as shown in FIG. 145, which illustrates the mechanism of dSFM transforming a sequence of spikes into analog activation via motor neuron. The dSFM signal, so (t), generated according to Eq. 6 in the range of [0, 100 mV] restores the polynomial curve. It is noteworthy as shown in FIG. 145 that the analog output of motor neuron signals is the result of the composition of a Fourier series in the interval [a, b]=[0, 10τ], i.e.:

$$\{ \begin{matrix}{s}_{0} ( t ) = {\sum }_{k = 0}^{∞} {A}_{k} sin ( \frac{π k t}{N} ) + {B}_{k} cos ( \frac{π k t}{N} ) , t ∈ [ a , b ] , N = \frac{b - a}{2} \\ {A}_{k} = \frac{1}{N} {\int }_{a}^{b} {s}_{0} ( t ) sin ( \frac{π k t}{N} ) d t , k \geq 0 \\ {B}_{k} = \frac{1}{N} {\int }_{a}^{b} {s}_{0} ( t ) cos ( \frac{π k t}{N} ) d t , k \geq 0 , {B}_{0} = \frac{1}{2 N} {\int }_{a}^{b} s ( t ) d t\end{matrix} \qquad ( 7 )$$

where the first six terms of the Fourier series is plotted that fit the given analog motor signals.

dSFM can be applied in brain-machine interfaces where neural signals are indirectly detected from outside of the brain particularly from the areas of the sensory, motor, visual cortexes and the conscious status memory embodied in the cerebellum.

Experiment 3. In a brain-machine interface, the externally detected neural signals resulting from dSFM in brain-machine interface as shown in FIG. 146 can be quantitatively explained as a set of dSFM signals generated by corresponding internal sequences of spikes. Each of the waveforms embodies a dSFM of an internal spike sequence.

Theorem 2. The principle of dSFM states that the external detection of a sequence of neural spikes is always in the form of a set of analog waveforms as the composition of a Fourier series.

Proof. Theorem 2 can be directly proved by Definition 4 and Eq. 7.

The dSFM theory is also empirically supported by Experiments 2 and 3, where the external detection of the sequences of neural spikes is always in the form of analog waveforms as a result of the dSFM, which is the composition of a Fourier serious of sine or cosine terms according to Eq. 7.

Theorem 1 and 2 as well as associated experiments have rigorously explained the fundamental questions raised in the beginning of this paper.

The sequence of spikes has been recognized as the fundamental means for neural signal representation and transmission in the nervous system. The semantics of spikes has been embodied by their sources and pathways based on the space divided mechanisms. The neural signaling theory of Spike Frequency Modulation (SFM) has explained the nature of neural signals and their transformation in the nerves systems of the brain. A set of mathematical models has been created in order to rigorously describe and manipulate neural signals towards brain machine interfaces and cognitive robots. The basic studies and theories have been supported by experiments and simulations towards applications in brain-machine interface systems and cognitive systems. They have also been applied to explain the neurological and cognitive foundations of artificial neural networks and brain-inspired systems.

It is to be noted that SFM and dSFM may be implemented in any type of computing device or computer system, such as shown in FIGS. 104, 110, 126, etc., with programming implementing SFM and dSFM.

An exemplary embodiment of a system 13900 that utilizes the FCU is shown in FIG. 139. The FCU is expressed differently at different levels of cognition, but has the same underlying mathematical properties, the unary system. System 13900 may include a processor/co-processor 13902, effectors 13904, write modalities 13906, read modalities 13908, sensors 13910, and databases 13912. Processor/co-processor 13902 may analyze FCU patterns in available read modalities 13908 and formulate stimulation signals to be applied through one or more write modalities 13906, completing the feedback loop. The analysis may be based on recorded an dynamically updated records in databases 13912, which may include, for example, patient records, disorder signatures, FCU expressions, etc. The analysis may choose the appropriate stimulation methods and signals.

Device Description

Embodiments may include a Medical Co-Processor (MCP) device which, using a variety of brain stimulation methods and sensors (read modalities), such as deep brain stimulation (DBS), electroencephalography (EEG) or ultrasound, provides series of signals to the brain or spinal cord and analyzes the response signals using analytical methods based on the Fundamental Code Unit (FCU), thereby decoding the patient, tissue and disorder-specific signal patterns. The FCU/MCP device then uses pre-determined or dynamically determined signatures to select treatment frequencies and sends signals to the targeted tissue via a variety of methods (write modalities) using effector devices such as enzymic controllers, optogenetic interfaces, or other signal carrier techniques to stimulate the cells for neural plasticity changes, specific protein switching/folding or electrochemical signaling sequences. The device therefore can be used for brain disorder diagnostics, and development of targeted treatment methods which activate the cells' internal resources.

Fundamental Code Unit and the Unitary System

In an embodiment, the Fundamental Code Unit (FCU), developed by Newton Howard (2012), is based on a mathematical construct known as the unitary system. Based on unary mathematics, this unitary system is clearly manifest in a number of physiological processes, including brain function and neuronal activity, molecular chirality and frequency oscillations within the brain. The unitary system is essentially a mechanism of spatiotemporal representation with a two-value (+“plus” or − “minus”) numerical system. At a synapse for instance, a neuron can release neurotransmitters that excite or inhibit another cell. The spectrum between these two poles, which is governed by the relative concentrations of each neurotransmitter, can be modeled according to these values since they bound the universe of discourse in this case. This system is used to represent many of the phenomena under the analytical purview of the Fundamental Code Unit, ranging from synapse activation and inactivation, to sensations of pain, to mind state calculations based on linguistic output.

Neural circuit perturbation can result from molecular as well as electromagnetic effects, causing changes in basal operation properties of local or global brain dynamics. Thus, interpreting the outcome of a causal neural circuit experiment included, but is not limited to, in the short term, the design of powerful control experiments, and in the longer term, radically better scaled methods for observing and influencing activity across the brain in order to understand the net neural impact of a perturbation.

One example application of the unary system is in the detection of peripheral nerve injury, which is a common cause of neuropathic pain. The presence of such pain suggests that the dynamic mapping of neural inputs and outputs has been altered. Using the unitary system, we can measure the aggregate of altered +/− inputs from healthy synapses. One of the areas of the brain implicated in pain perception, the Anterior Cingulate Cortex (ACC), consists of both inhibitory (−) and excitatory (+) neurons that respond to pain stimuli (tissue damage, temperature variations, etc.) in opposite manners. Inhibitory neurons cause action potentials to fire less, while excitatory neurons cause them to fire more. Persistent changes in synaptic strength such as long-term potentiation is observed in ACC synapses and in response to noxious stimuli, there is enhanced glutamate release and increase in AMPA receptor expression postsynaptically. This suggests that aggregates of inhibitions and excitations might be altered, thus modulating the unitary system due to synaptic strength changes.

Multi-Level/Multi-Modal Approach

The FCU/MCP's is a multi-level structure. In an embodiment, there are two fundamental categories of data streams to which we can access the unitary FCU value sequences, and later, to which we can assign diagnostic and clinical regimes. The first relates to activity within the brain (intracerebral). In an embodiment, this includes, but is not limited to, molecular signaling via chiral and protein-based neurotransmitters, as well as hormonal signaling and amine and peptide-based chemical signaling mechanisms. In an embodiment, the intracerebral level of analysis includes, but is not limited to, sub-molecular activity such as the production of specific synaptic proteins, such as neuropsin, resulting from increased electromagnetic activity (in the case of neuropsin, near-UV radiation in the 400-600 nm wavelength range). Finally, this layer includes connections between specific neurons and networks of neurons that may influence the manner in which specific cognitive events are manifested, such as memories (or lack thereof, as is the case in some forms of dementia).

The second relates to activity outside the brain, intracerebral activities are manifested behaviorally and linguistically. While these manifestations may appear to differ along cultural and geographical lines, the underlying neural processes driving them are identical, so they share the same underlying neural structure if not the same form. In terms of the FCU/MCP, behavior encompasses both voluntary and involuntary acts, since neurodegenerative diseases such as Parkinson's disease and Alzheimer's disease invoke uncontrollable behavioral changes. In addition to behavior, the extracerebral scope of FCU/MCP includes linguistic output as a means to determine mind state as well as cognitive faculty. In sum, the extracerebral realm of FCU/MCP is largely one of analysis and feedback, and the intracerebral component is an amalgam of analysis and clinical intervention. The primary distinguishing factor of FCU/MCP is thus the precise and holistic nature of the neural interventions and manipulations that take place. The importance of creating conceptual categories for each component of cognition relevant to FCU/MCP is that treatment modalities are created and employed in a manner that emulates these processes, rather than manipulates them using foreign chemical and electrophysical interventions.

Read modalities include a variety of ways in which a device can detect sequences of FCU values, and determine, for example, the potential effects of opening ion channels within the brain, as well as the expected changes to the conductance of these ions and protons beyond the immediate activation or silencing of cell. For instance, this might include, but is not limited to, the following:

long-term changes in neurons' storage of intracellular calcium

changes in the pH of neural nuclei in the brainstem

synaptically evoked neural spiking after photoactivation of neural ion pumps

rebound effects within neurons silenced by GABA(A) receptor inhibitors

Examining such less-studied effects such as these through the lens of novel read modalities helps lay conceptual groundwork for the second component of the FCU/MCP system: the write modality. FCU/MCP's write modality component includes both locally and remotely acting phenomena. In an embodiment, regarding local phenomena, optogenetic or pharmaceutical agents can be used to excite or inhibit specific neuron populations. Interventions, such as inhibitors and light-response treatment, promise to be significantly more effective at localized brain regions if the proper regions and cell networks can be identified. Neural modulators have a similar effect on neural network circuits, which means that the precise identification of vector networks for treatment delivery will likely be a significant component of future clinical neurology, with FCU/MCP taking the first steps toward that reality.

Stimulation or inhibition of brain activity using these methods essentially replicates what already happens to the brain in its natural form, but combined with read modalities, these methods will offer researchers and clinicians alike a uniquely precise methodology for targeting brain disorders. Studying potential downstream effects of specific types of brain activity and inactivity falls under the purview of the read modalities, and applying these methods to beneficially modifying brain processes is part of the write modalities.

Thus, for its read modality component, the FCU/MCP process flow is as follows: external observation->data acquisition->incorporation into FCU template->comparative analysis with other FCU templates at different levels of analysis->probabilistic diagnosis. Another key component of the system is the ability to use the FCU to compare an incomplete diagnostic picture (i.e., limited to a few external data streams) to previously collected data, including both healthy controls and patients with diagnosed disorders. This process promises to quicken the detection and identification of neurodegenerative disorders by reducing the amount and scope of data needed to make an authoritative diagnosis, as well as providing better access to existing information.

We expect the future of MCP research and applications to unfold in a rapid progression from further developing read modalities, to applying them to experimental write modalities, to finally applying both in the clinical realm. Further research will uncover more ways in which different patterns of stimulation within a region alter activity within that region, as well as how different patterns may differentially alter local or distal circuits. Precisely altered and balanced perturbations and neural pulse sequences, such as shuffle timings and shift timings, will then be used to determine how their effect can support clinical interventions for neurological and neurodegenerative disorders.

Example Modalities

The following subsections describe individual modalities, which may be used to read, write, or perform both functions as integral part of the FCU/MCP.

Neurotransmitter level and chirality measurement and control

Neurotransmitters are essential molecules at synapses that regulate brain, muscle and nerve function. The most common neurotransmitters are glutamate, dopamine, acetylcholine, GABA, and serotonin. At the cellular level, the FCU/MCP will build on neurotransmitter and receptor activation, because chemical synaptic transmission is one of the primary ways by which neurons communicate with one another.

For instance, ligand-substrate interactions, which are a prerequisite for biochemical reactions that are relevant to cognition, are governed primarily by neurotransmitter molecules and provide an ideal example of the potential to employ FCU/MCP as a feedback-based write modality. These molecules exist in one of two forms, each being a molecular mirror image of the other. Isomer-enantiomer ligands function as lock-and-key allowing neurotransmitters to recognize their complementary receptors and permit excitatory or inhibitory synaptic transmission. Mirror image isomer/enantiomers interact with post-synaptic receptor sites, a process that produced a variety of effects depending on environmental conditions. The specific ligand-substrate characteristics, or lock mechanism, required for neurotransmitter activity, are determined by the unique electron-level interactions between asymmetric molecules. Chiral neurotransmitter molecules are found in S(+) or R(−) isomer-enantiomer conformations and have different effects on neural activity and behavior. For example, the S (+) isomer is several times more potent than its R (−) enantiomer. The S (+) isomer is known to induce euphoria, whereas enantiomer R (−) has been linked to depression. The overall greater potency of the S (+) isomer form in such cases suggests that this form may have a higher potential for deep cranial stimulant actions and neurotransmitter availability in the synapse. This leads to behavioral alterations that are noticeable at the corresponding linguistic level. The correlations between the linguistic output and S (+) isomer and R (−) enantiomer values offer corresponding equivalence of transporter's chemical pathways, allowing correlation with other FCU/MCP's read modalities, such as linguistic analysis.

Recent findings indicate that neurotransmitters can be measured using a Fast scan cyclic voltammetry. Measuring and modulating neurotransmitter levels provides a solid treatment approach for subjects with a variety of disorders. Treatment for regulating neurotransmitter levels is to provide the basic amino acid precursors in order to maintain adequate neurotransmitter levels. In this sense, measuring neurotransmitters and drug treatments provide “read” and “write” modalities respectively for analyzing FCU.

Electrochemical Neural Manipulation

Photon-driven conformational changes in protein neurotransmitters form one of the primary mechanisms by which information is transferred and stored within the brain. Apart from controlling the concentration and neural regions affected by controlled neurotransmitter release or inhibition, electromagnetic radiation can be used to a similar effect, by inducing conformational changes in the proteins already present near the synapse site of neurons.

A powerful write modality can be built using FCU-based mechanism for exchanging information within the brain: endogenous photon-triggered neuropsin transduction, followed by conformational changes in protein neurotransmitters. By mimicking the causal process by which the brain writes new information to neural networks, FCU/MCP can co-opt existing chemical processes to achieve control over this activity.

In a neuropsin-mediated unary-coded photonic signaling scheme, neuropsin plays a role of a unary +/− encoder, capable of producing patterns of LTP in synaptic ensembles, and wiring changes in local synaptic circuits. Both phenomena may be reflective of, and serve as a coded reporter of, each of neuropsin's two stable conformational states: i.e., incremental unary (+/−) switches based on value structure of a non-deterministic state, with or without linear or potential pathway. The incremental unary “+” switch is near UV photon absorption by neuropsin, producing its incremental unary “+” state which is G-protein activation. The incremental unary “−” switch is blue (˜470 nm) photon absorption, which converts into the conformation incapable of G-protein activation.

Multiphoton absorption by neuropsin may be possible, if neuropsin is in close proximity to a photon source, therefore free radical reactions can generate photons of longer wavelength, >600 nm. Multiphoton absorption of two or more of such (red) photons can provide energy equivalent to that of a single UV photon; this means that if two red photon absorptions occur, it may also serve as the incremental unary “+” switch, substituting for a single UV photon. An advantage of longer wavelength photons is that they travel longer distances in brain tissue than do UV photons.

Other key regulatory enzymes, like NADPH oxidases (NOXs), may be used to create such incremental unary switches. Flavoproteins like NOXs absorb blue photons, which cause them to emit green photons. Like NAD(P)H, it's autofluorescent, but is higher on the wavelength spectrum. The photons which NOXs absorb are the same photons that the UV-stimulated NAD(P)H emits: ˜470 nm (blue). These photons trigger the production of photons of even longer wavelength, by NOXs' well-documented ability to autofluoresce: 520 nm green photons are emitted.

Quantally controlled, unary incremental switches in the brain may use a multiplicity of other (+/−) switches in the brain, as NOX's photonic (+/−) unary coding may serve as switches for yet another regulatory process, such as reactive free radical generation, which produces UV photons that start the scheme, involving NADH, neuropsin in the first place. Therefore, NOX can complete the photonic scheme of the brain's infinite “do loop”, reaching quantum tunneling & entanglement, which open the door for long-distance signaling, even from outside the brain.

Downstream consequences of neuropsin's ability to produce spatio-temporal distribution patterns of “+” and “−” states in synaptic domains are potentially profound, in their implications for memory formation, both short- and long-term, each of which are semi-independent processes.

Long term: There exists a link between long-term memory (LM) and cellular/synaptic processes such as long-term potentiation/depression (LTP/LTD). Furthermore, LTP/LTD requires some sort of structural changes/protein synthesis:

1. changing neurotransmitter receptor expression,

2. increasing synapse size,

3. changing synapse anchoring, that makes ADP/ATP, being the major energy source in neurons and glial cells, required for LM.

Short term: There is good evidence that persistent neuronal firing of those populations of neurons that encode the memory is required, similarly to refreshing computer's rapid-access memory. Apart from ATP/ADP fueling persistent activity by driving ATP/ADP dependent ionic pumps and the maintenance of synaptic receptors, ATP/ADP has also been linked directly to the emergence of persistent activity through its modulation of ATP modulated potassium channels.

Since the discovery of purinergic signaling the involvement of ATP/ADP-mediated signaling through neuronal and glial receptors is seen in almost every aspect of brain function. FCU/MCP, can guide purinergic signaling, including its effects on learning and memory, focused more on the therapeutic potential of purinergic modulation in various CNS disorders.

Linguistic Analysis

The FCU/MCP approach is based on the concept that cognition, or thoughts, are composed of similar units. Within the brain, thought can be measured, or quantified, based on brain locality, the amount and source of neurotransmitters and other intervening chemicals, as well as pre-existing conditions in the brain that might cause different responses to the same neural stimuli. Outside the brain, linguistic and behavioral patterns can be observed that can be causally traced to these lower-level processes. Because of this fundamental linkage, FCU consists not of just one of these metrics, but is instead a relational quantifier for all of them, and each such unit must account for the various sources of conscious thought. For example, reasoning calls upon events in both long and short-term memory, in addition to applicable learned concepts. Information regarding each of these may appear based on its manifestation to be retrieved, stored and modified differently within the brain, but at the most basic, indivisible level, this information is composed of similarly formatted units.

We can think of language as a function that maps those chemical and cellular processes within the brain to some meaningful expression. To a lesser degree, behavior also fits this definition. Because language is inextricably bound to processes inside the brain, it is a valuable window with which to examine the inner workings of the brain, which is why FCU/MCP's read modalities include linguistic analysis, to map the processes that ultimately lead individuals to express specific behaviors or linguistic expressions.

Linguistic processing is primarily viewed as a read modality, analyzing spoken or written discourse. However, one can also envision applications which in the short term, may propose the use of specific concepts and language constructs in communications with a patient, and in the long term, using language in write modality capacity by the FCU/MCP device capable of automated cognitive therapy.

Functional Magnetic Resonance Imaging (fMRI)

Conventional neurofeedback “read” modality techniques such as electroencephalography (EEG) provide signals that are too noisy and poorly localizable. An improvement in the imaging signal is offered by fast and localizable source signal provided by real-time functional magnetic resonance imaging (fMRI). The temporal resolution of fMRI is in the scale of seconds or less while the spatial resolution is in the scale of millimeters. It has been shown that healthy individuals can use fMRI to learn to control activity in their brain. Recent research has shown that patients with pain disorders can control brain areas involved in pain perception using fMRI-neurofeedback. This self-regulation of brain activity is brought about in the following manner: The subject is in the MR scanner visualizing a signal during which fMRI imaging is performed which is the “read” modality. During the “write” modality, the neurofeedback signal is computationally adjusted. The subject visualizes neuro signal changes in brain regions which is fed back into the signal the subject views.

Visual perceptual learning (VPL) in the early visual cortex of adult primates is sufficiently malleable that fMRI feedback can influence the acquisition of new information and skills when applied to the correct region of the brain. Second, these methods can induce not only the acquisition of new skills and on formation but can aid in the recovery of neurological connections that have been damaged by accident or disease. For instance, a trauma victim suffering from language skill loss can potentially recover those skills through fMRI neurofeedback induction. The structure of thought is that the FCU, which we seek in cognition, must be based on some finite number of neurological connections. These same connections are influenced by the activity of fMRI neurofeedback. This process does not target a single neuron, but a locality of connected neurons, and based on its positive effects on the conscious process of VPL, the FCU represents that reality. In addition, fMRI induction research can provide powerful evidence for the composition of thought because it can be used to determine the minimum amount of neuronal connectivity for the formation of thoughts.

Electroencephalography (EEG)

Techniques such as fMRI are used to detect brain activity, however, the temporal resolution presently available is not good enough for determining unitary math at the cellular level. For this purpose we propose that electroencephalography (EEG) can be used. EEG has better temporal resolution (milliseconds vs. seconds and minutes of fMRI) and it is non-invasive. EEG can be used as a “read” modality to allow measurement of FCU at the cellular level.

EEG allows recording electrical activity in the brain from neurons that emit distinct patterns of rhythmic electrical activity. The aggregate of synchronous neural activity from a large group of neurons emit rhythmics patterns. Different EEG rhythms are associated with normal or abnormal brain activity. There are seven unique frequencies of brain waves (from low to high): delta, theta, alpha, beta, gamma. Each set of frequencies is associated with a brain state such as alertness, sleep, working memory etc.

Conventional EEG tends to have excellent temporal resolution, but it is the poor spatial resolution that makes it difficult to localize important brain activity. High resolution EEG (HREEG) is also a non-invasive technique used to evaluate brain activity based on scalp potential measurements. HREEG is used to enhance spatial resolution over regular EEG by overcoming the head volume conductor effect. One type of HREEG is cortical potential imaging (CPI). CPI allows passive conducting components of the head to deconvolve scalp potential. This powerful spatio-temporal EEG “read” modality will allow to record localized and stimulus specific brain activity.

Transcranial magnetic stimulation (TMS)

TMS is another non-invasive technique that can cause neurons to become activated by depolarization or silenced by hyperpolarization. TMS utilizes electromagnetic induction that results in generating electric currents using a magnetic field resulting in activation in a specific brain areas. TMS can be used as a diagnostic tool or for therapy. TMS has been used for the treatment of depression and schizophrenia among others.

TMS can be used as a “write” modality to feedback activation of neurons that require an increase in excitability or silence neurons that are hyperexcitable.

Deep Brain Stimulation (DBS)

Deep brain stimulation, or DBS, is a surgical treatment that requires the implantation of a brain pacemaker that sends electrical activity to specific brain regions. DBS has most commonly been used in the treatment of Parkinson's disease, other movement disorders, depression, and chronic pain. Unlike brain lesioning methods of neurological treatment, DBS treatment is reversible.

DBS is primarily useful as a “write” modality for the treatment of chronic diseases such as movement disorders, as it is an invasive technique. The method by which DBS affects neural activity and neurotransmitters is still largely unknown, but it produces high frequency electrical stimulation that reduces neurological disease symptomatology. In some cases, DB S activates ATP release that acts on adenosine receptors and inhibits neural activity therefore mimicking a lesioning effect.

Audiovisual Stimulation (AV)

Audio-visual sources can be used as a neurostimulation input used during neurofeedback. Audio inputs produce signals through the auditory neural pathway for perception of sounds and visual neural inputs activate the visual pathway for perception of light. When audio-visual input is presented to individuals, the correlated brain activity can be measured by the above described techniques. Once the neural activity is measured, inputs are processed into a “writeable” form that is fed back into the audio-visual program.

Ultrasound (USN)

Ultrasound (USN) has recently been shown to non-invasively stimulate brain activity. USN has the capability to increase or decrease neuronal activity, thus making it an ideal candidate for novel neurofeedback applications. One kind of USN is the transcranial pulse ultrasound that has the key advantage of spatial resolution of a few millimeters. Transcranial ultrasound has been shown to disrupt seizure activity in a mouse model of epilepsy. Recent technological advances now allow transmitting and focusing of USN through the intact human skull using an array of phase-corrected ultrasonic transducers placed on the cranium. Such non-invasive, focused ultrasonic intervention permits thermal (high power) and non-thermal (low-power) modes. Non-invasive, thermal ablation of thalamic nuclei using USN has recently been demonstrated to be effective in the treatment of neuropathic pain patients, and promises applicability in non-thermal stimulation and suppression of neural activity.

Motion Tracking/Gait Analysis

The vestibular system, which is located primarily in the mesencephalon and receives input from proprioception receptors from throughout the body, is another promising perspective from which to assess brain function relative to protein folding and misfolding. Since it is integrated with input from the cerebellum, semicircular canals and visual and auditory system and relays information and coordinates the motor system to maintain balance, the vestibular system is responsible for maintaining motion equilibrium. Since this system serves keep the body sensitive to perturbations in the surrounding environment, neurogenic disorders affecting this system are largely marked by motion aberrations that can be detected by multiple body sensors, creating another rich read modality.

Analytical Methods

Brownian Motion Based Analysis

The analytical component of FCU/MCP's will also be based in part on the phenomenon of Brownian motion in order to probabilistically analyze the effect of environmental factors such as electrical charge, the presence of other reactive neurochemicals, and ambient electromagnetic energy. Brownian motion measures particle displacement as proportional to the square root of time elapsed. That is, measuring from a hypothetical time t₀=0, displacement d of some Brownian particle will increase in proportion to \sqrt{t} rather than t due to the random forces acting upon the particle. Modeling the impact of many random forces that tend to cancel one another's influence (but not always) is significant to the FCU for a number of reasons. First, the conformational changes in the fluoroproteins that drive the neurochemical element of the FCU must account for some degree of randomness in the incidence of UV energy causing those conformational changes, as well as the chemical energy that is released when they occur. Whereas Brownian motion is used as a stochastic predictive model to describe and account for the uncertainty inherent in particle motion when numerous fast-moving particles interact with one another without any kinetic coherence, the process can be applied to protein-driven neurotransmission as well.

In Brownian motion, a set of particles is described with a series of properties affecting the outcome, such as mass, direction, speed, and interactions with other particles. Over the set of all particles, these factors appear to cancel one another out instead of contributing to a general pattern of motion, as may appear when water travels in one direction (such as in the direction of gravity). In human cognition, we can substitute these attributes for what is observable within the human brain. For instance, instead of describing the motion of particles in a fluid, we can use a similar model to describe the state of protein receptors located on neurons in a specific brain region. Instead of identifying a pattern of motion versus a random state, our approach searches for a pattern of cognitive process versus the absence of such a pattern, as might occur when comparing neurochemical patterns from healthy patients and those with cognitive impairments.

In sum, the greatest applicability of Brownian motion and other stochastic mathematical models to the FCU is the ability to measure “background noise,” and to identify some threshold at which a series of neurons is producing such noise or producing an information-rich signal.

Linguistic Axiological Input/Output (LXIO) Analysis

The LXIO (Linguistic Axiological Input/Output) System, developed by Howard and Guidere (2012), is an existing computational analysis suite for evaluating mind state according to observable cues, such as spoken and written language, that is based on unary mathematical principles. This system forms an integral component of MCP by expressing cognitive states in terms of axiology, or the common unary values associated with certain general concepts, such success and failure. Axiological elements such as conception, perception, and intention are taken into consideration. The overall LXIO framework consists of multiple modules, each of which retrieves, parses, or processes patient discourse and/or writing. The framework for our analytics engine consists of multiple modules responsible for coherently and systematically retrieving, parsing, and processing a patient's discourse. The LXIO modality consists of a computational method that can analyze with numerous processes simultaneously, and is based on the mind-state indicator (MSI) algorithm. The MSI algorithm was developed to explain mental processes that underlie human speech and writing in order to predict states of mind and cognition. The MSI algorithm is covered in patent application Ser. No. 13/083,352, “Method for Cognitive Computing”.

The MSI algorithm can detect mood states in individuals by evaluating word value information from their speech based on cultural and linguistic norms. Speech information is derived from concepts such as semantic primitives, which tend to have universal conceptual value. Death, for instance, has a generally negative value across cultures and languages, whereas concepts such as rest and happiness have positive values. MSI takes into account both the content and the context (vocal, body and semantic) in each conceptual primitive. That means both a comparison of words to known values and expressions to known mind states, such as consistent body language (folding arms, touching face etc.) or vocal tonality (pitch variations correlated with levels of expressiveness, as well as volume and word emphasis).

Markov Decision Process (MDP)

Viewing cognition as a mapping of one set of phenomena to another, it is easy to over-emphasize its spatial components at the expense of its temporal construct. Since cognition is a dynamic process heavily dependent on the environment, the units we use to describe and interpret thought must reflect its temporality. FCU/MCP uses the concept of mind state, or an approximation of the human mind or some subset of it at any point in time. Mapping the temporality of thought requires the connection of several such mind states over time, which are themselves composed of FCU units. In order to develop the relationship between the FCU and temporality, FCU/MCP uses the Markov Decision Process model to build mind state transitions through reasoning and decision-making. This analytical process forms the foundation for the two linked goals of FCU/MCP: the empirical and predictive analysis of cognitive information, as well as the modification of brain processes to alter that information.

Cognitive processes depend on their current state. That is, information from the past, if not already contained in the process's current state, will not contribute to greater precision or informational clarity of the process. For that reason, we use as the basis of our analysis a process flow model known as the Markov chain, which is the building block of the Markov Decision Process (MDP). The MDP is unique in its ability to allow decision makers to evaluate and act on incomplete information, or in the presence of some uncertainty.

Since states of mind evolve and change over time, then each change has probabilistic characteristics that can be placed at various points on a one-dimensional spectrum between explicitly positive or explicitly negative. Based on this probabilistic property of mind state transitions, there is also a range of therapeutic, or manipulative, interventions that depend on that probability. The means by which we measure the efficacy of such treatment is based on the responses of the patient throughout treatment and/or experimentation, and the positive or negative values which those responses connote.

We can describe this process in a straightforward manner. When in some mind state s, there is some probability p, where 0<p<1, that the subject will shift to a new mind state, s, with some benefit b. Markov chains, in our application, consist of a series of such shifts. The process of thought can be thought of as a sequence of some number of distinct states over a period of time, and the process can be modeled based on the probability of transition from one state to the next. These transition probabilities depend on n previous states and nothing more. For our purposes, n is generally set to 1 in order to bound our analysis to the current state and its successor.

For example, if we have a MDP for some four different mind states {S0, S1, S2, S4}, from each mind state there is a possibility of choosing an action from the set {a0, a1 . . . an}. When that action is chosen and executed, the subject assumes the successive mind state. Thus we have two components: potential decision (the choice of an action in a given state), and transition probabilities for each decision node. Finally, these transitions can generate rewards based on the positivity or negativity of the resulting mind state.

In order to fully and effectively map mind states to probabilistic transitions, it is important to develop a sub-model that accounts for processes within the brain, such as the activation of specific neurons or neural networks in response to chemical stimuli. To this end, an algebraic component can be introduced in order to account for increasingly numerous concept and brain region activations. Beginning with an set S (infinite for our purposes here) representing brain regions that are candidates for activation, a σ-algebra A on that set can be then introduced, with elements a∈A known as activation sets. Note that by definition, a⊂S. Another set W is then introduced, with elements as labeled concepts in the brain that correspond to conceptual constructs. For some subset of A there exists a mapping P: a∈A→w−W, or the concept activation mapping. The elements of this subset are action potentials. Thus, there is some mapping P:∈W→ã∈Ã be a mapping we call the brain activation mapping. From this mapping, we can determine the probability of state transitions because brain region activation/inactivation is the most immediate cause of mind state change. If μ is some measure on S, then F:A→{+,−} is a parity mapping. An axiology, which we use to link linguistic information to brain region activation information in our FCU analysis, is a mapping Ξ: W→{+,−} generated by computing f(w)=ₐ F(s)^{d(μ)} with a=P(w). We then project Ξ(w)=sig(n(f)) for the final result.

Using this system, we can interpret data relating to the mind state of a subject by examining the mind's abstract structures: axiological concepts expressed in language, as well as periods of brain region activity and inactivity. These structures are populated by information from present read modalities, ranging from simple observation to biopsy and long-term analysis. Throughout the brain there are various forms of activations (electrical, chemical, biological) each contributes individually or within groups to the formation of new concepts, which define a positive or negative mental state.

Maximum Entropy (Maxent) Statistical Model

The Maximum Entropy (Maxent) statistical model is of high significance to the FCU/MCP. The Maxent Model is a method of estimating conditional probability. In the case of FCU/MCP, the core equation can be used, H(p)=−Σ ˜p(x)p(y|x)log p(y|x), as a component of both the read and write modality because each of these is influenced by probabilistic events.

Given the expanded Maximum Entropy equation:

$${L}_{\tilde{p}} ( p ) ≡ log {\prod }_{x , y}^{ } {p ( y \vert x )}^{\tilde{p} ( x , y )} = {\sum }_{x , y}^{ } \tilde{p} ( x , y ) log p ( y \vert x )$$

The following data is obtained:

X: input value (can consist of any elements which can influence the results; also note that x is a member in the set of X.)

Y: output value; note that y is a member in the set of Y.

P (y|x): entire distribution of conditional probability

˜p(x,y): empirical probability distribution

˜p(x,y)=1/N*number of times that the pair (x,y) occurs in the sample

f(x,y): The expected value of f with respect to the empirical distribution ˜p(x,y) is precisely the statistic we use to measure probability of state transition and activation probability. This gives us ˜p(f)=Σ˜p(x)p(y|x)f(x,y). Solving for p(f)=˜p(f) then yields Σ˜p(x)p(y|x)f(x,Y)=Σ˜p(x,y)f(x,y).

In natural language processing (NLP), Maxent essentially means assigning a probability to each possible meaning of a given word that is being processed. For instance, in the English language the word produce can have at least two meanings: as a verb, it means to generate or create (meaning 1), and as a noun it generally refers to agricultural harvest and output (meaning 2). If we assume that these are the only nontrivial uses of the word, then p(Meaning 1)+p(Meaning 2)=J. While this is a highly simplified example that does not address the probability distributions within each meaning (such as the fact that it is much more likely to be used in the verb form), it does provide a basic framework that can be expanded to account for increasingly complex linguistic constructs.

A stochastic model is a model that represents the behavior of the seemingly random process of NLP when fed unstructured information. They employ a series of five templates, and construct probability distributions for each of them by employing constraints based on context, source language, and destination language. For instance, “template 1” has contains the loosest set of constraints, since a distinct target language is not specified and there is likely no morphological change. However, templates 2-5 perform translation based on syntactic context, verb proximity, and verb character. A stochastic model's relevance to FCU/MCP is its distinction between probability and determinism in conceptual constructs. In an ideal setting, FCU-based analysis links each unit to another one intuitively, and there is very little (if any) uncertainty that the FCU that maps to processes within the brain accurately reflects those processes. Here, the model is much less certain and must account for the idiomatic differences between languages. While FCU as a theoretical method does not face this problem because linguistics are simply an outer layer of a much deeper series of cognitive activities, imperfections in data gathering may provide a viable application for such a model in our research. For instance, garbled speech (thanks to recording hardware, data corruption, or human error) may create a set of unknown and known words in a single sentence, and the context of the known words must be used to create a Maxent model for the potential unknown word matches.

Another possible use of a Maxent model is predictive analysis. Given a mind state correlated with a series of spoken concepts, future behavior (depressive vs. non-depressive) and linguistics (attributable to cognitive state) can be discerned to a reasonable measure of certainty using Maxent. In the context of MCP, a number of contextual templates could be designed based on variables such as mind state (+/−), or temporality (i.e., whether the concepts discussed refer to past or future events). This is because multiple concepts that occur in the same temporal frame are likely to be related.

From the above research we can discern a number of Maxent uses within the FCU/MCP. The first is the use of statistical methods to determine the most likely intended conceptual meaning of homophones such as produce, or rose. Researchers previously applied Maxent to sentence content, meaning that the Maxent solution to a sentence containing rose would vary based on the presence or absence of other concept words such as flower, petal, or red in the first meaning or seats, standing, or seated in the second meaning. FCU would apply maxent in a similar manner, but would consider input from a multitude of sources. For instance, the presence of hand gestures associated with certain activities, such as rising from one's seat, would figure in the FCU-based read modality analysis of a sentence containing rose. In addition, the normalized mind state associated with flower(s), if significantly different from background, would also contribute to the final determination of the word's meaning and consequent connection with conceptually and semantically adjacent words and ideas. Maxent can also be applied to mind-state and linguistic tendencies of individuals and sets of individuals who share some cognitive similarity, such as Post-traumatic stress, Parkinson's disease, or Alzheimer's disease.

A template-based Maxent model algorithm for predictive read modality analysis might look like this:

```
Process_1(string s, concept set S)
Given SENTENCE
  Get WORD COUNT
If s(0), s(1) belong to concept, merge(s(0),s(1))
else remove(s(1), s)
process(string s, concept set S)
Process_2(concept set S)
  FOR each concept in S
    Get temporality
    Get mind state
    Get set of possibly related concepts in order of probability
```

This presents just one simple template based on temporality and mind state, two factors which we know will affect the physical execution of cognition within the brain based on chemical activation and/or brain region activation. Maxent can be applied to determine the probability that a given neural network may be activated at certain combinations of temporality and mind state, but that will likely require significant data gathering on the individual beforehand.

### Example Embodiment

Embodiments may include (1) one or more Sensors each implementing at least one read modality, (2) an Analyzer comprised of commodity hardware parts, whose primary purpose is to provide data look-ups in a pre-loaded database containing FCU templates for different read and write modalities, and perform FCU computations on them, recognizing patterns provided in input, and create therapeutic signal pattern, and (3) one or more Effectors, reconfigurable at runtime to efficiently deliver signal sequences.

The Analyzer is connected via Interface 1 to an array of Sensors. The Sensors are used to perform functions like examining areas of brain tissues, collect the frequencies of neuronal activity, or aggregate linguistic and behavioral information of the patient, and transmit them to the Analyzer for processing.

Interface 2 connects the Analyzer to one or more Effectors used to stimulate targeted neural tissue, in order to induce and guide brain activity. The Effectors are devices that can deliver signal to the targeted neural tissue via invasive (e.g., implanted optical probes) or non-invasive methods (e.g., transcranial stimulation).

The Analyzer, via Interface 2, controls the Effectors to induce neuronal activity feedback, which is collected via Sensors from Interface 1 as a series of action potential spikes or linguistic patterns, ultimately represented as a stream of unitary system values. The Analyzer, using this input, isolates a set of FCU templates, such as the baseband oscillation frequencies specific to the area of activity, and matches them to a set of unitary system signals which can be delivered via a write modality, to induce electro-chemical release sequences, in turn triggering specific protein switching/folding sequences in the cells.

The Analyzer, via Interface 2, dynamically reconfigures the Effector to produce the required sequences of signals, which are delivered to the brain. The signals activate changes such as the release of a specific set of positive (+) or negative (−) optical isomers of chemicals in the tissue. The chemical communications triggered by the isomer release activates tissue changes in the targeted area.

To better understand the embodiment, below is an example of FCU/MCP device used for treatment of Alzheimer's disease symptomatics:

In the case of Alzheimer's disease, the device would use several read modalities collected using a Multi-modal Body Sensor Network (mBSN), such as Howard and Bergmann (2012), consisting of multiple sensor types: an Integrated Clothing Sensor System (ICSS) to measure knee joint stability and arm trajectory, and a vocal data collector linked to the linguistic analysis engine to detect and analyze mind states and temporal delays based on spoken language. Analyzing movements of both the upper and lower limbs provides empirical evidence regarding mind state (e.g., as a proxy for uncertainty), which can be coupled to linguistic and behavioral output for a richer diagnostic picture of early Alzheimer's patients. Motion information from patients that are likely to develop Alzheimer's disease is collected in terms of (+) and (−) terms: involuntary movements, like in Myoclonus, that are sudden and brief, can be classified as (+) or (−). (+) movements are caused by sudden muscle contractions, while (−) movements are caused by sudden loss of muscle contractions. Similarly, mind state information collected is in the form of +/− connotations to words suggesting +/− mind states. Data collected from Sensors are then sent via Interface 1 to the Analyzer.

In the Analyzer, using stored FCU models, computes these unitary values of +/− and also computes the treatment strategy. The treatment strategy is delivered into Effector through Interface 2, which in this case implements the Ultrasound modality, which in turn delivers drugs to treat Alzheimer's symptomatology. In the case of Alzheimer's disease, the FCU model computes delivery of +/− isomers of anticholinesterase, the drug commonly used to treat Alzheimer's disease but is typically given intravenously. The novelty of this treatment strategy is using FCU to deliver the drug by 1) choosing enantioselective (+/−) versions of anticholinesterase for drug delivery, 2) using the “write” modality of Ultrasound to deliver in a more precise manner the drug directly to neurons affected by Alzheimer's. The manner in which this would work is as follows: ultrasound beam targets the hippocampus, which is heavily implicated in controlling memory and is affected by early Alzheimer's disease. The ultrasound beam opens up a temporary drug delivery passage in the blood brain barrier with the help of microscopic bubbles in intravenously injected that travel to brain capillaries. There are several anticholinesterases, such as phenserine and rivastigmine both of which have enantiomers. Phenserine in addition to inhibiting cholinesterases, is able to modulate beta-amyloid precursor protein (APP) levels. Interestingly, phenserine has differing actions of its enantiomers: (−)-phenserine is the active enantiomer cholinesterase inhibition, while (+)-phenserine, also known as posiphen has weak activity as an cholinesterase inhibitor and can be given at high concentrations. It is important to note for Alzheimer's treatment that both enantiomers are equipotent in reducing APP levels.

In order to treat Alzheimer's disease symptomatology based on FCU, the Analyzer selects the best fit enantiomer of anticholinesterase and utilize (+)-posiphen, either alone or in combination with (−)-phenserine delivered directly into the hippocampus attenuate the progression of Alzheimer's disease at an early stage. In this manner of treatment, memories stored in the hippocampus will not be lost.

Applications

Early diagnosis of neurodegenerative disorders

The effects of neurodegenerative disorders such as Parkinson's disease and Alzheimer's disease can ultimately be alleviated, or at least minimized, by the development of an accurate, non-invasive early detection mechanism complementary to that of linguistic analysis that is based on behavioral trends over time. Thus, part of FCU/MCP development will include current research and expand on recent findings by validating a non-invasive diagnostic methodology for the early detection of Parkinson's disease. Specifically, the integration of body sensor networks will provide a physical dimension to FCU/MCP's read modality. Multi-modal Body Sensor Networks (mBSN) consist of multiple sensor types: an Integrated Clothing Sensor System (ICSS) to measure knee joint stability and arm trajectory, and in the future a vocal data collector linked to the LXIO analysis engine to detect and analyze mind states and temporal delays based on spoken language.

By focusing our efforts towards early detection of changes in global cognitive and postural functioning during everyday life, our research promises to provide a direct match with the symptoms that define this disease. The mBSN approach to early detection is especially effective and appropriate in cases where patient risk is too low to warrant surgical intervention, but where a patient nevertheless requires some level of clinical care or observation. In these cases, write modalities could simplify the patient's choice about whether to treat a given disorder based on the low complication risk owing to the precision of FCU/MCP write modalities.

Body Sensor Networks (BSN) offer a new way to collect data during the performance of everyday tasks involving physical movements. Body Sensor Network data for broad categories of activity, including standing, walking, and repetitive tasks that will enable rapid subject dataset growth, will be used to measure values linked to the onset of neurodegenerative diseases, such as joint instability and erratic arm trajectories. Analyzing movements of both the upper and lower limbs offers the chance to collect empirical evidence regarding mind state, which can be coupled to linguistic and behavioral output for a richer diagnostic picture of the subject.

Alzheimer's Disease

The well-known chemical symptoms of neurological disorders such as Alzheimer's disease often manifest themselves too late for treatment to sufficiently slow or reverse the onset of the disorder. The current research emphasis on early detection, preventive lifestyle adjustment, and pharmaceutical intervention presupposes that noninvasive methods either will not work, or that doctors are simply unable to detect the disease in time to effectively apply those treatments. To this end, FCU/MCP system seeks to apply methods of improved early detection in order to more effectively apply “write modalities” such as the introduction of chemical inhibitors of the beta-amyloid proteins that build up within the brain and cause Alzheimer's disease.

We can use a similar model to introduce constraints on the brain regions we measure. In patients with Alzheimer's disease, increased presence of hyperphosphorylated tau protein aggregates and amyloid senile plaques are telltale neurobiological signs of the disorder. We know the effect of tau proteins and plaques at the individual neuronal level, and thus can extrapolate those effects so that they match what is observed in patients with Alzheimer's. Because their cognitive faculties appear less orderly than those of healthy patients, dementia patients tend to exhibit more neurological chaos, or randomness, that doesn't contribute to coherent thought or linguistic output. FCU/MCP device can apply Brownian motion analysis to the affected brain regions, neural networks, and individual neurons, and use this method to predict the coherence of a patient's mind state. This may in turn help us to better define the thresholds at which certain types of cognitive tasks, such as memory recall and language processing, begin to be affected by dementia onset, and the tolerance of healthy cognition for such levels of random activity in the brain.

For disorders such as Alzheimer's disease, symptoms of the disease include cognitive deficiency and memory loss; biomarkers include indicators found in cerebrospinal fluid, as well as genetic factors and the presence of abnormal levels of beta-amyloid proteins in the brain. However, a true “read modality” cannot be limited to symptomatic analysis based on these factors alone.

The approach is based on using the Fundamental Code Unit (FCU) to perform pattern recognition tasks on the linguistic and behavioral data emerging from observations of a patient. Data streams can be as unobtrusive as recording a spoken interview or observing changes in gait over several years' time, and as invasive as collecting cerebrospinal fluid. Data from each of these acquisition methodologies are then incorporated into the FCU template. While FCU is a brain language of sorts, it is fundamentally different from spoken languages in two ways. First, languages such as English map spoken words (utterances) and/or written (pictorial) representations to cognitive constructs; translators then draw equivalencies between English and other languages. The FCU incorporates characteristics of both. It is similar to a “language” of cognition because it is applicable to all intelligent, brain-based entities. It is similar to a translator because it draws the same type of equivalencies between molecular processes, such as an increase in beta-amyloid proteins, and physically observable processes, such as uncertain gait and slurred language.

The FCU/MCP's selection of write modalities depends largely on the biomarkers present and the progression of the disorder that is detected. For instance, an ideal treatment for Alzheimer's disease would both slow the BA protein buildup in the brain and reverse the cognitive effects that have already begun to appear. In the absence of a clinical treatment to reverse the effect of beta-amyloid protein buildup in the brain, early detection of Alzheimer's disease is the most popular management regime.

For the latter component of the treatment, a “write modality” for Alzheimer's disease is necessary that will reconstruct the connections between neurons that provided the basis for now-missing memories. In order for this to be possible, some means of relating missing neural information to what is readily available is needed. The FCU can contribute to symptomatic (and causal factor) reversal by reconstructing partial neural connections from extrapolation of incomplete FCU data, combined with linguistic and behavioral data streams. While the clinical technology does not yet exist to apply these innovations to patients, a robust means for both cataloging and relating different neural data streams, or FCU, is a necessary prerequisite.

Parkinson's Disease

Mental states are the manifestations of particular neural patterns firing and neurotransmitters exchanged between neurons. These states have neural correlations corresponding to specific electrical circuits. A decade ago there was a deep interest in functional neurosurgery for neural disorders, such as movement disorders as well as neurodegenerative cognitive impairment. This led to an increase in our understanding of the underlying neural mechanisms and circuitry involved in basal ganglia disorders with improved surgical techniques and the development of deep brain stimulation (DBS) technology, which paved the way for major advances in the treatment of Parkinson's Disease (PD) and other neurological disorders.

To better understand the role of the posterior parietal cortex, basal ganglia and cerebellum in the control of movement, researchers inserted electrodes into patients with movement disorders such as Parkinson's disease (PD). These electrodes helped stimulate the control network system (CNS) for which low frequency (4-15 Hz) field potentials were recorded that correlated with the patient's involuntary movements. Interestingly, recent studies have discovered that the pedunculopontine nucleus (PPN) in the upper brainstem has extensive connections with several motor centers in the CNS and is very important in controlling proximal muscles for posture and locomotion.

This area is over-inhibited in many patients, which is a major cause of their inability to move, i.e. in an akinesia state. This inhibition can be overcome by stimulating the PPN directly and can thus return previously chair-bound patients to a useful life. That is why, Deep Brain Stimulation (DBS) of the pedunculopontine nucleus (PPN) is a novel neurosurgical therapy developed to address symptoms of gait freezing and postural instability in Parkinson's disease and related disorders.

FCU/MCP based diagnosis will offer improved and early detection of PD symptoms and provide effective treatment strategies. Similar to Alzheimer's patients, but more importantly for a movement disorder such as Parkinson's, motion information can be collected from Sensors such as body sensor networks (mBSN) (Refer to FIGS. 131, 132). Motion data is collected from patients that are likely to develop Parkinson' disease is collected in terms of unary (+) and (−) terms: involuntary movements, like in Myoclonus, that are sudden and brief, can be classified as (+) or (−). (+) movements are caused by sudden muscle contractions, while (−) movements are caused by sudden loss of muscle contractions. This information is sent to the FCU based Analyzer that computes unary treatment strategies based on unary biomarkers of Parkinsonian movement symptoms. Again, similar to Alzheimer's ultrasound can be used a write modality to deliver drugs into PD associated brain regions delivering enantioselective phenserine and posiphen (same drugs can be used for both AD and PD).

Pain Detection and Management

Chronic pain affects approximately 25% of the U.S. population. Chronic pain is classifiable according to two types: neuropathic pain and nociceptive pain. Neuropathic pain is caused by damage to the nervous system, and is described as a “burning, tingling, shooting, or lightning-like” pain. Examples include neuralgia, complex regional pain syndrome, arachnoiditis and postlaminectomy pain, which is residual pain following anatomically successful spine surgery and a common indication for neurostimulation therapy. Compared to nociceptive pain, neuropathic pain is more severe, more likely to be chronic, and less responsive to analgesic drugs and other conventional medical management.

Nociceptive pain originates from disease or tissue damage outside the nervous system, and it can be dull, aching, throbbing, and sometimes sharp. Examples include bone pain, tissue injury, pressure pain and cancer pain. Nociceptive pain is caused most directly by peripheral nerve fiber stimulation, and is classified as such because the causes of nociceptive pain generally have at least the potential to harm body tissue.

Current objective diagnostic procedures for chronic pain include imaging techniques such as computed tomography (CT), magnetic resonance imaging (MRI) and intramuscular electromyography (EMG). CT and MRI provides information about anatomic abnormalities, but are expensive and do not give information about pain type or intensity level. EMGs provide objective evidence of nerve dysfunction. However, these strategies are invasive and often painful. Newer objective pain detection methods include, quantitative sudomotor axon reflex test (QSART) and autonomic function “hot/cold” pain detection test. Although these methods are effective in research labs, they are difficult to use in clinical settings, often require special training, and are hard to bill for. What is needed is an objective measure that detects the presence or absence of pain as well as an objective assessment of pain intensity level that the patient is feeling.

Neuropathic pain arises from damaged neural tissues that can be essential when the neural injury is in the brain or spinal cord. In patients with intractable central neuropathic pain the pain seems to be caused by spontaneous oscillations in the ‘central pain matrix’ which consists of the periaqueductal gray, peri-ventricular gray (PAG/PVG), globus pallidus, thalamus, anterior cingulate, insula and the orbitofrontal cortex. It was found that driving the PAG/PVG by stimulating at 10 Hz, one can eliminate the oscillations and reduce the patients' feelings of pain very considerably. Pain suppression is frequency dependent and pain relief occurred at PVG simulation levels ranging from 5-25 Hz. There are also correlations between thalamic activity and chronic pain. This low frequency potential may provide an objective index for quantifying chronic pain, and may hold further clues to the mechanism of action of PVG stimulation.

While it has been widely discussed that specific frequencies affect neural tissue functioning and development, the mechanisms guiding this effect have not been found. Understanding how frequencies affect the complex electrochemical structures and processes in neural tissue, and being able to determine the ranges and sequences that aid and/or restore normal neural activity, are seen as the next step in addressing neurological disorders. Furthermore, non-neural cells are driven by electrochemical processes and can be subjected to similar treatments.

Current neuropathic pain management strategies either require surgery or pharmacotherapy. Surgical strategies are invasive and often require nerve stimulation or destruction of nerve cells. These invasive techniques often cause even more damage to the nervous system which can enhance the pain level. Additionally, none of the surgical techniques have been found to be uniformly successful in managing neuropathic pain. Pharmacotherapy is not efficacious and could have many side effects. In some cases, multiple drugs are necessary for optimize pain level and insufficient data exists for combination drug therapy for neuropathic pain. Transcranial direct current stimulation (tDCS) or TMS can be used as a write modality. TDCS permits weak current stimulation of specific areas of the brain to increase or decrease brain wave patterns as needed for specific treatments. It has been shown that tDCS and TMS can be used to reduce fibromyalgia pain. In this manner, DBS or HD-EEG can be used as read modality and tDCS or TMS can be used as write modality to both diagnose and manage chronic pain using FCU/MCP.

Deep Cell Stimulation

Cell growth is one of the primary results of the cell cycle, and can be accelerated or slowed by a variety of factors. Growth factors work to promote both cell differentiation and maturation, and these processes can in turn be manipulated to promote or decelerate the growth of cell mass. Many cytokine regulator proteins, for instance, work to increase the growth rate of hematopoietic and immune system cells. Some of these, such as Fas ligands, are used to program cells to destroy themselves at pre planned intervals. Still other growth factors are communicable by ever-circulating proteins suspended in body fluid, and work by binding to surface receptors on the target cell.

In much the same way that neurons can be activated or inactivated by neurotransmitters, cells can self-destruct, accelerate growth, or slow growth based on chemical messengers and growth factors. To harness this ability for scientific or clinical ends requires a thorough understanding of the “language” in which cells communicate with one another hormonally. FCU/MCP provides a framework that can be applied not only to the biology of cognition, but to physiology itself. Specifically, we already know that FCU/MCP can be harnessed in order to manipulate specific neurons and neuron networks by using a read modality to interpret their signals and a write modality to modify them. A very similar methodology can be applied to injury and disease victims by manipulating cell growth to regenerate lost tissue, or restrict the growth of malignant cells. Deep Cell Stimulation (DCeS), along with the diagnosis and treatment of brain disorders, is one of the most promising applications of the FCU/MCP framework since it applies to so many clinical disorders, including osteoporosis, hypohemia, and traumatic injuries such as broken bones and injured skin.

Unique Social and Long Term Consequences

FCU represents a potential paradigm shift in Artificial Intelligence, both in its facilitation of cognitive analysis and cognitive manipulation. Apart from the gains to be made by structuring AI to match the physiological and physical attributes of intelligent cognition as we currently know it more closely, there are a number of other potential advances with profound social implications.

By bridging the structural gap between “artificial” and “real” intelligence, the capacity for these intelligences to interact with one another becomes much more realistic. This also means that AI can be used as a cognitive bridge between human intelligences that were previously linked by comparatively crude methods (read: spoken and written language). The development of the FCU on a large scale thus has a number of wide-ranging effects. First is the potential to obviate language. The core of the FCU concept is the notion that, regardless of what happens at the syntactic layer of linguistic output, it can be ultimately traced to physical, and biochemical processes within the brain. Since these processes are identical among humans, achieving the ability to read thoughts, emotions, mind states, and intentions at this low level has the potential to change the way humans interact.

If we imagine that the FCU has in fact transformed the way people communicate in this way, there are certain features we can expect to see in society and at the individual level. Psychotherapy will begin to resemble streaming content from Netflix as interfaces develop that can transmit massive amounts of cognitive information with minimal latency. In fact, a “psychologist” may in fact be a synthetic intelligence or network of such entities. Since information sharing in this case would no longer depend on the ambiguities of linguistic idiom, native tongues, or nonverbal expressions. Since specific stimuli (dreams, fantasies, horror, etc.) are composed of the same FCU units as baseline conscious thought, the sensations evoked by each of these could be provided without going to the movies, watching TV, reading or even experiencing the stimuli firsthand.

One of the more disconcerting features of a society such as this one that has transcended the linguistic and cultural differences that language barriers pose is the ability to replicate an entire “brain image;” that is, the sum of an individual's experiences, actions, and memories that contribute to the individual persona. While this may appear positive due to the ability to “back up” a consciousness, the notion that making a full, downloadable copy of a human life begs some serious questions about privacy and individual liberties. For instance, could a person be “copied” unwittingly and have their analytical faculties put to use without their consent? Surely data mining and advertising companies would find ways to exploit this newfound intimacy with the human psyche at the individual level. In 1984, Orwell wrote that even living under the most intellectually and culturally repressive regimes, one still remained the master of what remained inside his/her brain. With the ease of potentially surreptitious access to the brain, even Orwell may have been too optimistic.

On the other hand, the ability to copy and distribute an individual's cognitive identity may allow great strides in therapeutic treatments for neurodegenerative disorders. Diseases such as Alzheimer's, for instance, work by slowly eroding the neural connectivity between brain regions until memories, skills, instincts and other aspects of one's identity bound to their brain matter disappear. If the disease is detected sufficiently early, it may be possible to recover the majority (or even totality) of what is all too often inevitably lost to these diseases. Connections within the brain could then be reconstructed based on clinical researchers' knowledge of the precise mechanisms causing a given neurodegenerative symptom (i.e. a lack of sufficient connectivity between brain region a and brain region b).

Regarding communication itself, knowledge of the FCU can be applied to create and analyze the same cognitive structures that appear in language, such as metaphors, idioms, and figures of speech. However, since the underlying conceptual content is laid bare, the utility of these constructs may decrease, as we are increasingly able to apply the FCU to problems of translation and analysis. Linguistic analysis engines that are FCU-based need not collect data on chemical and physical phenomena within the brain in real time. Instead, a statistical analysis of the FCU's role in phenomena such as anger, depression, and deceit (and the underlying processes that drive them) can be correlated with the audiovisual data available, including speech, mind state, and nonverbal expressions. As more FCU data are collected through thorough experimentation, the analytical engine becomes more accurate, and the ideal of a “universal translator” becomes more realistic.

The ability to copy high-fidelity cognitive engrams has a variety of additional applications relating to the ability to “live” or “re-live” specific experiences, possibly in a manner different than they actually occurred. In the therapeutic realm, sufferers of PTSD and similar disorders may undergo therapy regimes that return them to the traumatic experiences that are the cause of their disorder. In addition, “re-living” experiences may alter the way justice is sought, with witnesses being able to trace specific experiences and examine them with a clarity that may have been lost in a fog of adrenaline and other hormones, especially if the experience was a traumatic or intense one.

The above predictions only presuppose the ability to “read” FCU information from the human brain. The ability to write it inside the human brain may yet be realized, and if it is, the collective notions of individuality, soul and reality will likely be fundamentally altered. The ability to erase memories, create new ones, and essentially construct a human psyche from the ground up (instincts, habits, tendencies, preferences, and even personality traits) may tempt some to attempt creating the “perfect” human, much like the eugenics movement of the early 20^{th} Century. In addition, since cognitive factors such as those listed above are hypothetically alterable, people may elect to have themselves altered in order to conform with standards or expectations set by society at large. In addition, knowing what little we do about the effect of such re-writing on the brain itself, there may be no limit on the number of times a person can be “re-written,” and we have no way of knowing at what point a person ceases to assume their former identity and assumes a new one.

Another implication of the ability to “write” to the human brain in the natural FCU language of the brain is to manufacture increasingly accurate predictions of the future. Using the Intention Awareness concept, the ability to acquire FCU information from relevant actors will make models of causality and social activity forecasts significantly more accurate and useful to decision makers.

In a future where neuroscience and AI are largely governed by the discovery of the FCU, we can also expect the emergence of new data storage methodologies, since the FCU is essentially a filesystem for the brain. Data connectivity, as it is today, will still remain an important of the future computational infrastructure, but data storage and transfer will less resemble the transfer of sequences of bits than the exchange of much smaller bits and pieces of data, since the human brain is more capable of extrapolation than current computational hardware/software. Given the right data “seeds,” FCU sequences can likely be reproduced without the whole data stream.

As shown in FIGS. 104, 126, and 130, the present invention contemplates implementation on a system or systems that provide multi-processor, multi-tasking, multi-process, and/or multi-thread computing, as well as implementation on systems that provide only single processor, single thread computing. Multi-processor computing involves performing computing using more than one processor. Multi-tasking computing involves performing computing using more than one operating system task. A task is an operating system concept that refers to the combination of a program being executed and bookkeeping information used by the operating system. Whenever a program is executed, the operating system creates a new task for it. The task is like an envelope for the program in that it identifies the program with a task number and attaches other bookkeeping information to it. Many operating systems, including Linux, UNIX®, OS/2®, and Windows®, are capable of running many tasks at the same time and are called multitasking operating systems. Multi-tasking is the ability of an operating system to execute more than one executable at the same time. Each executable is running in its own address space, meaning that the executables have no way to share any of their memory. This has advantages, because it is impossible for any program to damage the execution of any of the other programs running on the system. However, the programs have no way to exchange any information except through the operating system (or by reading files stored on the file system). Multi-process computing is similar to multi-tasking computing, as the terms task and process are often used interchangeably, although some operating systems make a distinction between the two.

The present invention may be a system, a method, and/or a computer program product at any possible technical detail level of integration. The computer program product may include a computer readable storage medium (or media) having computer readable program instructions thereon for causing a processor to carry out aspects of the present invention. The computer readable storage medium can be a tangible device that can retain and store instructions for use by an instruction execution device.

The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry (such as that shown at 208 of FIG. 2) may include, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Although specific embodiments of the present invention have been described, it will be understood by those of skill in the art that there are other embodiments that are equivalent to the described embodiments. Accordingly, it is to be understood that the invention is not to be limited by the specific illustrated embodiments, but only by the scope of the appended claims. Further, it is to be noted that, as used in the claims, the term coupled may refer to electrical or optical connection and may include both direct connection between two or more devices and indirect connection of two or more devices through one or more intermediate devices.

What is claimed is:

1. A method for neural stimulation comprising:
receiving electrical and optical signals from electrophysiological neural signals of neural tissue from at least one read modality, wherein the electrophysiological neural signals are at least one of Spike frequency modulated or Spike frequency demodulated;
encoding the received electrical and optical signals using a Fundamental Code Unit;
automatically generating at least one machine learning model using the Fundamental Code Unit encoded electrical and optical signals;
generating at least one optical or electrical signal to be transmitted to the brain tissue using the generated at least one machine learning model, wherein the generated signals are at least one of Spike frequency modulated or Spike frequency demodulated; and
transmitting the generated at least one optical or electrical signal to the neural tissue to provide electrophysiological stimulation of the neural tissue using at least one write modality.

2. The method of claim 1, wherein the received Spike frequency modulated signals are obtained from sensory neurons.

3. The method of claim 2, wherein the generated Spike frequency modulated signals are generated using signal transform function that converts an analog stimulus on a sensory neuron into a sequence of spikes, wherein the rate of spikes per second (sps) is proportional to the intensity of the input.

4. The method of claim 3, wherein the generated Spike frequency modulated signals have a rate of 0 to 100 spikes per second and an amplitude of 0 to 100 mV.

5. The method of claim 1, wherein the received Spike frequency demodulated signals are obtained from motor neurons.

6. The method of claim 5, wherein the generated Spike frequency demodulated signals are generated using a left rectangular numerical integration of the SFM signals for each sampling period determined by a given threshold of conversion.

7. A system for neural stimulation comprising:
at least one read modality adapted to receive electrical and optical signals from electrophysiological neural signals of neural tissue, wherein the electrophysiological neural signals are at least one of Spike frequency modulated or Spike frequency demodulated;
at least one write modality adapted to transmit the generated at least one optical or electrical signal to the neural tissue to provide electrophysiological stimulation of the brain tissue; and
at least one computing device comprising a processor, memory accessible by the processor, and program instructions stored in the memory and executable by the processor to cause the processor to perform:
encoding the received electrical and optical signals using a Fundamental Code Unit;
automatically generating at least one machine learning model using the Fundamental Code Unit encoded electrical and optical signals; and
generating at least one optical or electrical signal to be transmitted to the neural tissue using the generated at least one machine learning model, wherein the generated signals are at least one of Spike frequency modulated or Spike frequency demodulated.

8. The system of claim 7, wherein the received Spike frequency modulated signals are obtained from sensory neurons.

9. The system of claim 8, wherein the generated Spike frequency modulated signals are generated using signal transform function that converts an analog stimulus on a sensory neuron into a sequence of spikes, wherein the rate of spikes per second (sps) is proportional to the intensity of the input.

10. The system of claim 9, wherein the generated Spike frequency modulated signals have a rate of 0 to 100 spikes per second and an amplitude of 0 to 100 mV.

11. The system of claim 7, wherein the received Spike frequency demodulated signals are obtained from motor neurons.

12. The system of claim 11, wherein the generated Spike frequency demodulated signals are generated using a left rectangular numerical integration of the SFM signals for each sampling period determined by a given threshold of conversion.

13. A computer program product comprising a non-transitory computer readable storage having program instructions embodied therewith, the program instructions executable by a computer system, to cause the computer system to perform a method of neural stimulation comprising:
receiving electrical and optical signals from electrophysiological neural signals of neural tissue from at least one read modality, wherein the electrophysiological neural signals are at least one of Spike frequency modulated or Spike frequency demodulated;
encoding the received electrical and optical signals using a Fundamental Code Unit;
automatically generating at least one machine learning model using the Fundamental Code Unit encoded electrical and optical signals;
generating at least one optical or electrical signal to be transmitted to the brain tissue using the generated at least one machine learning model, wherein the generated signals are at least one of Spike frequency modulated or Spike frequency demodulated; and
transmitting the generated at least one optical or electrical signal to the neural tissue to provide electrophysiological stimulation of the neural tissue using at least one write modality.

14. The computer program product of claim 13, wherein the received Spike frequency modulated signals are obtained from sensory neurons.

15. The computer program product of claim 14, wherein the generated Spike frequency modulated signals are generated using signal transform function that converts an analog stimulus on a sensory neuron into a sequence of spikes, wherein the rate of spikes per second (sps) is proportional to the intensity of the input.

16. The computer program product of claim 15, wherein the generated Spike frequency modulated signals have a rate of 0 to 100 spikes per second and an amplitude of 0 to 100 mV.

17. The computer program product of claim 13, wherein the received Spike frequency demodulated signals are obtained from motor neurons.

18. The computer program product of claim 17, wherein the generated Spike frequency demodulated signals are generated using a left rectangular numerical integration of the SFM signals for each sampling period determined by a given threshold of conversion.
